(19) United States
(12) Patent Application Publication
(10) Pub. No.: US 2013/0312130 A1
(43) Pub. Date: Nov. 21, 2013

(54) SOMATIC CELL-DERIVED PLURIPOTENT CELLS AND METHODS OF USE THEREFOR

(71) Applicants: Douglas Dean, Prospect, KY (US); Yongqing Liu, Louisville, KY (US)

(72) Inventors: Douglas Dean, Prospect, KY (US); Yongqing Liu, Louisville, KY (US)

(73) Assignee: UNIVERSITY OF LOUISVILLE, Louisville (US)

(21) Appl. No.: 13/976,879

(22) PCT Filed: Sep. 23, 2011
(86) PCT No.: PCT/US11/53012

§ 371 Date: Jun. 27, 2013

Related U.S. Application Data
(63) Continuation of application No. 12/951,678, filed on Nov. 22, 2010.
Application No. 12/951,678 is a continuation-in-part of application No. PCT/US2009/067503, filed on Dec. 10, 2009.
(60) Provisional application No. 61/201,420, filed on Dec. 10, 2008.

(51) Int. Cl. C12N 5/074; A61D 19/04

(52) U.S. Cl. CPC C12N 5/0696; A61D 19/04
USPC 800/18; 435/377; 435/357; 435/366; 800/14; 424/93.7; 600/34

(57) ABSTRACT

Provided are methods for producing a reprogrammed fibroblast or epithelial cell. The methods include growing a plurality of fibroblasts or epithelial cells in monolayer culture to confluency; and disrupting the monolayer culture to place at least a fraction of the plurality of fibroblasts or epithelial cells into suspension culture under conditions sufficient to form one or more embryoid body-like spheres, wherein the one or more embryoid body-like spheres comprise one or more reprogrammed fibroblasts or epithelial cells that express one or more markers not expressed prior to the disrupting step. Also provided are reprogrammed fibroblasts or epithelial cells produced by the disclosed methods, formulations that include reprogrammed fibroblasts or epithelial cells, methods for using the reprogrammed fibroblasts or epithelial cells, methods for producing chimeric non-human mammals that include one or more sphere-induced Pluripotent Cells (siPS), and chimeric non-human mammals produced thereby.

### CROSS REFERENCE TO RELATED APPLICATIONS

This application claims priority to U.S. patent application Ser. No. 12/951,678, filed Nov. 22, 2010, the disclosure of which is incorporated herein by reference in its entirety.

### GRANT STATEMENT

This invention was made with government support under grant EY018603 awarded by the National Institutes of Health. The government has certain rights in the invention.

### TECHNICAL FIELD

The presently disclosed subject matter relates to reprogrammed somatic cells. Particularly, the presently disclosed subject matter provides reprogrammed somatic cells, methods for generating reprogrammed somatic cells, and uses for reprogrammed somatic cells. The presently disclosed subject matter also relates to chimeric mice comprising reprogrammed somatic cells, and methods of producing the same.

### BACKGROUND

It has long been believed that the development of the cells, tissues, and organs of animals results from an orderly progression of differentiation events from stem cells to terminally differentiated cells. This progression has been thought to be unidirectional, starting with the earliest totipotent cells found in the early stage embryo to the ultimate, terminally differentiated cells that make up the vast majority of the adult animal.

This paradigm has been challenged recently by reports that certain differentiated somatic cells can be “reprogrammed” to what appears to be an earlier stage of development (i.e., a more pluripotent state) by introducing expression vectors that encode polypeptides associated with pluripotency into the cells. For example, it has been shown that both mouse and human fibroblasts can be reprogrammed to form embryonic stem (ES) cell-like cells by the recombinant expression of four transcription factors: Oct4, Sox2, Klf4, and c-Myc (Takahashi & Yamanaka, 2006; Takahashi et al., 2007). These cells have been referred to as “induced pluripotent stem cells” (iPSC), and have been shown to express certain stem cell markers, to form teratomas, and even to give rise to germline-competent chimeric mice when injected into blastocysts (see Maherali & Hochedlinger, 2008). Thus, it appears that differentiation might not be exclusively unidirectional, and at least some degree of pluripotency can be reacquired by cells otherwise believed to be terminally differentiated.

Unfortunately, recombinant DNA techniques have certain disadvantages for reprogramming cells, particularly with respect to cells that are to be administered to subjects. For example, many expression vectors that are commonly used for expressing exogenous nucleic acids such as those that might induce reprogramming are based on retroviruses. Retroviral expression vectors have been shown to be characterized by significant safety issues, most notably increased incidences of cancer resulting from the introduction and subsequent integration of the vectors into the cells of subjects to whom the retroviral vectors had been administered.

What are needed, then, are methods for reprogramming somatic cells to reintroduce some degree of pluripotency desirably without the need to resort to the use of recombinant expression constructs, particularly in the form of retroviral constructs. This need, among others, is addressed by the presently disclosed subject matter.

### SUMMARY

This Summary lists several embodiments of the presently disclosed subject matter, and in many cases lists variations and permutations of these embodiments of the presently disclosed subject matter. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

The presently disclosed subject matter provides methods for producing a reprogrammed fibroblast or epithelial cell. In some embodiments, the methods comprise (a) growing a plurality of fibroblasts or epithelial cells in monolayer culture to confluency; and (b) disrupting the monolayer culture to place at least a fraction of the plurality of fibroblasts or epithelial cells into suspension culture under conditions sufficient to form one or more embryoid body-like spheres, wherein the one or more embryoid body-like spheres comprise a reprogrammed cell (e.g., a reprogrammed fibroblast or epithelial cell) comprising expressing one or more markers not expressed by a cell growing in a monolayer culture prior to the disrupting step. In some embodiments, the fibroblast or epithelial cell is a mammalian fibroblast or epithelial cell, optionally a human fibroblast or epithelial cell. In some embodiments, the fibroblast or epithelial cell is a non-recombinant fibroblast or epithelial cell. In some embodiments, the disrupting comprises scraping the confluent monolayer off of a substrate upon which the confluent monolayer is being cultured. In some embodiments, the methods further comprise maintaining the one or more embryoid body-like spheres in suspension culture for at least one month. In some embodiments, the one or more embryoid body-like spheres are maintained in a medium comprising Dulbecco's Modified Eagle Medium (DMEM) and 10% fetal bovine serum (FBS). In some embodiments, the reprogrammed fibroblast or epithelial cell expresses a stem cell marker selected from the group consisting of Oct4, Nanog, fibroblast growth factor-4 (FGF4), Sox2, Klf4, SSEA1, and Stat3.

In some embodiments, the presently disclosed methods further comprise replating the embryoid body-like spheres produced under conditions sufficient for the reprogrammed fibroblasts or epithelial cells present therein to form colonies. In some embodiments, the conditions sufficient comprise plating the embryoid body-like spheres on a fibroblast feeder layer in an embryonic stem cell medium until colonies of sphere-induced Pluripotent Cells (siPS) are produced. In some embodiments, the presently disclosed methods further comprise subcloning one or more cells present in a colony of reprogrammed fibroblasts or epithelial cells to form one or more sphere-induced Pluripotent Cell (siPS) cell lines

The presently disclosed subject matter also provides reprogrammed fibroblasts or epithelial cells produced by the disclosed methods.

The presently disclosed subject matter also provides reprogrammed fibroblast or epithelial cells non-recombinantly induced to express one or more endogenous stem cell markers.

The presently disclosed subject matter also provides formulations comprising the disclosed reprogrammed fibroblast or epithelial cells in a pharmaceutically acceptable carrier or excipient. In some embodiments, the pharmaceutically acceptable carrier or excipient is acceptable for use in humans.

The presently disclosed subject matter also provides embryoid body-like spheres comprising a plurality of reprogrammed fibroblasts or epithelial cells.

The presently disclosed subject matter also provides cell cultures comprising the disclosed embryoid body-like spheres in a medium sufficient to maintain the embryoid body-like spheres in suspension culture for at least one month.

The presently disclosed subject matter also provides methods for inducing expression of one or more stem cell markers in a fibroblast or epithelial cell. In some embodiments, the methods comprise (a) growing a plurality of fibroblasts or epithelial cells in monolayer culture to confluency; and (b) disrupting the monolayer culture to place at least a fraction of the plurality of fibroblasts or epithelial cells into suspension culture under conditions sufficient to form one or more spheres, wherein the one or more spheres comprise a reprogrammed fibroblast or epithelial cell expressing one or more stem cell markers. In some embodiments, the methods further comprise replating the spheres formed under conditions sufficient for one or more reprogrammed fibroblasts or epithelial cells present therein to form one or more colonies. In some embodiments, the conditions sufficient for one or more reprogrammed fibroblasts or epithelial cells present therein to form colonies comprise culturing the replated spheres in the presence of an embryonic stem cell medium at least until one or more cells derived from the replated spheres form one or more colonies.

The presently disclosed subject matter also provides methods for differentiating a reprogrammed fibroblast or epithelial cell into a cell type of interest. In some embodiments, the methods comprise (a) providing an embryoid body-like sphere comprising reprogrammed fibroblast or epithelial cells; and (b) culturing the embryoid body-like sphere in a culture medium comprising a differentiation-inducing amount of one or more factors that induce differentiation of the reprogrammed fibroblast or epithelial cells or derivatives thereof into the cell type of interest until the cell type of interest appears in the culture. In some embodiments, the cell type of interest is selected from the group consisting of a neuronal cell, an endodermal cell, and a cardiomyocyte, and derivatives thereof.

In some embodiments, the cell type of interest is a neuronal cell or a derivative thereof. In some embodiments, the neuronal cell or derivative thereof is selected from the group consisting of an oligodendrocyte, an astrocyte, a glial cell, and a neuron. In some embodiments, the neuronal cell or derivative thereof expresses a marker selected from the group consisting of glial fibrillary acidic protein (GFAP), nestin, β III tubulin, oligodendrocyte transcription factor (Olig) 1, and Olig2. In some embodiments, the culturing is for at least about 10 days. In some embodiments, the culture medium comprises about 10 ng/ml recombinant human epidermal growth factor (rhEGF), about 20 ng/ml fibroblast growth factor-2 (FGF2), and about 20 ng/ml nerve growth factor (NGF).

In some embodiments, the cell type of interest is an endodermal cell or derivative thereof. In some embodiments, the culturing comprises culturing the embryoid body-like sphere in a first culture medium comprising Activin A; and thereafter culturing the embryoid body-like sphere in a second culture medium comprising N2 supplement-A, B27 supplement, and about 10 mM nicotinamide. In some embodiments, the culturing in the first culture medium is for about 48 hours. In some embodiments, the culturing in the second culture medium is for at least about 12 days. In some embodiments, the endodermal cell or derivative thereof expresses a marker selected from the group consisting of Nkx6-1, Pdx 1, and C-peptide.

In some embodiments, the cell type of interest is a cardiomyocyte or a derivative thereof. In some embodiments, the culturing is for at least about 15 days. In some embodiments, the culture medium comprises a combination of basic fibroblast growth factor, vascular endothelial growth factor, and transforming growth factor β1 in an amount sufficient to cause a subset of the embryoid body-like sphere cells to differentiate into cardiomyocytes. In some embodiments, the cardiomyocyte or derivative thereof expresses a marker selected from the group consisting of Nkx2-5/Csx and GATA4. In some embodiments, the embryoid body-like sphere is prepared by (a) growing a plurality of fibroblasts or epithelial cells in monolayer culture on a tissue culture plate to confluency; and (b) disrupting the monolayer culture to place at least a fraction of the plurality of fibroblasts or epithelial cells into suspension culture under conditions sufficient to form one or more embryoid body-like spheres, wherein the one or more embryoid body-like spheres comprise a reprogrammed fibroblast or epithelial cell.

The presently disclosed subject matter also provides methods for treating a disease, disorder, or injury to a tissue in a subject comprising administering to the subject a composition comprising a plurality of reprogrammed fibroblast or epithelial cells in a pharmaceutically acceptable carrier, in an amount and via a route sufficient to allow at least a fraction of the reprogrammed fibroblast or epithelial cells to engraft the tissue and differentiate therein, whereby the disease, disorder, or injury is treated. In some embodiments, the disease, disorder, or injury is selected from the group consisting of an ischemic injury, a myocardial infarction, and stroke. In some embodiments, the subject is a mammal. In some embodiments, the mammal is selected from the group consisting of a human and a mouse. In some embodiments, the methods further comprise differentiating the reprogrammed fibroblast or epithelial cells to produce a pre-determined cell type prior to administering the composition to the subject. In some embodiments, the pre-determined cell type is selected from the group consisting of a neural cell, an endoderm cell, a cardiomyocyte, and derivatives thereof.

The presently disclosed subject matter also provides methods for isolating sphere-induced pluripotent cells (siPS). In some embodiments, the presently disclosed methods comprise (a) growing a plurality of fibroblasts or epithelial cells in monolayer culture on a tissue culture plate to confluency; and (b) disrupting the monolayer culture to place at least a fraction of the plurality of fibroblasts or epithelial cells into suspension culture under conditions sufficient to form one or more embryoid body-like spheres; (c) replating the spheres formed on a fibroblast feeder layer in an embryonic stem cell medium; (d) culturing the replated spheres on a fibroblast feeder layer in an embryonic stem cell medium for a time sufficient for colonies of undifferentiated siPS derived from the replated spheres to develop; and (e) isolating the siPS from one or more of the colonies. In some embodiments of the presently disclosed methods, the siPS are mouse siPS and the embryonic stem cell medium is a mouse embryonic stem cell medium comprising leukemia inhibitory factor (LIF), or the siPS are human siPS and the embryonic stem cell medium is a human embryonic stem cell medium comprising basic fibroblast growth factor (bFGF).

The presently disclosed subject matter also provides methods for producing a chimeric animals including, but not limited to chimeric mice. In some embodiments, the methods comprise transferring one or more sphere-induced Pluripotent Cells (siPS) into a host embryo, implanting the host embryo into a recipient female, and allowing the host embryo to be born, wherein a chimeric animal comprising one or more somatic and/or germ cells that is/are (a) progeny cell(s) of one or more of the siPS transferred into the host embryo is produced. In some embodiments, the one or more siPS is/are produced by (a) growing a plurality of fibroblasts or epithelial cells in monolayer culture to confluency; and (b) disrupting the monolayer culture to place at least a fraction of the plurality of fibroblasts or epithelial cells into suspension culture under conditions sufficient to form one or more embryoid body-like spheres. In some embodiments, the one or more embryoid body-like spheres comprise a reprogrammed fibroblast or epithelial cell induced to express at least one endogenous gene not expressed by a fibroblast or epithelial cell growing in the monolayer culture prior to the disrupting step. In some embodiments, the disrupting comprises scraping the confluent monolayer off of a substrate upon which the confluent monolayer is being cultured. In some embodiments, the methods further comprise maintaining the one or more embryoid body-like spheres in suspension culture for at least one month. In some embodiments, the one or more embryoid body-like spheres are maintained in a medium comprising DMEM and 10% FBS. In some embodiments, the reprogrammed fibroblast expresses at least one endogenous gene is selected from the group consisting of Oct4, Nanog, FGF4, Sox2, Klf4, Ssea1, and Stat3. In some embodiments, the methods further comprise replating the embryoid body-like spheres under conditions sufficient for the reprogrammed fibroblasts or epithelial cells present therein to form colonies. In some embodiments, the conditions sufficient comprise plating the embryoid body-like spheres on a fibroblast feeder layer in an embryonic stem cell medium until colonies of sphere-induced Pluripotent Cells (siPS) are produced. In some embodiments, the methods further comprise subcloning one or more cells present in a colony of reprogrammed fibroblasts to form one or more sphere-induced Pluripotent Cell (siPS) lines. In some embodiments, the fibroblast or epithelial cell comprises at least one transgene. In some embodiments, the transgene is operably linked to a promoter that is active in at least one cell type and/or developmental stage of a chimeric animal that comprises a siPS derived from the fibroblast or epithelial cell to an extent sufficient to modify a phenotype of the chimeric animal as compared to a non-chimeric animal of the same species and/or genetic background as that of the host embryo into which the siPS were introduced. In some embodiments, the transferring comprises transferring at least six siPS into the host embryo and/or the implanting comprises implanting the host embryo into a pseudopregnant female animal. In some embodiments, the host embryo is a morula stage embryo or a blastocyst stage embryo.

The presently disclosed subject matter also provides in some embodiments chimeric animals including, but not limited to chimeric mice, produced by the presently disclosed methods. In some embodiments, the chimeric animals are pre-term embryos. In some embodiments, one or more sphere-induced Pluripotent Cells (siPS)-derived cells are present within the germline of the chimeric animal, thereby producing a germline chimeric animal.

Thus, it is an object of the presently disclosed subject matter to provide chimeric animals comprising siPS-derived cells.

An object of the presently disclosed subject matter having been stated herein above, and which is achieved in whole or in part by the presently disclosed subject matter, other objects will become evident as the description proceeds when taken in connection with the accompanying drawings as best described herein below.

### BRIEF DESCRIPTION OF THE DRAWINGS

The instant application file contains at least one drawing executed in color. Copies of this patent or patent application publication with color drawing(s) will be provided by the United States Patent and Trademark Office upon request and payment of the necessary fee.

FIGS. 1A-1I are a series of photographs showing that sphere formation triggered stable changes in the morphology of cells with disruptions in the three RB1 family genes RB1, RBL1, and RBL2 (referred to herein as “triple knockout cells” or TKOs).

FIG. 1A shows TKOs at passage 4 in monolayer culture. FIG. 1B shows TKOs lacked contact inhibition and formed mounds after reaching confluence in culture. FIG. 1C shows outgrowth of mounds, such as those shown in FIG. 1B, subsequently led to detachment from the plate and sphere formation. FIG. 1D shows TKO spheres two weeks after transfer to a non-adherent plate. FIG. 1E shows central cavity formation (arrow) evident in TKO spheres after several weeks in suspension culture. FIG. 1F shows that TKO spheres formed in suspension culture reattached when transferred back to tissue culture plates, and all cells in the spheres migrated back onto the plate to reform a monolayer. FIG. 1G shows a higher power view of the boxed region in FIG. 1F. FIG. 1H shows monolayers of sphere-derived cells two days after spheres were transferred back to a tissue culture plate. FIG. 1I shows cells in FIG. 1H after one week in culture. Note that cells in FIGS. 1H and 1I had diverse morphologies, and further that they were smaller than TKO cells prior to sphere formation (FIG. 1A).

FIGS. 2A and 2B are photographs of TKO cells (FIG. 2A) and TKO-Ras cells (TKO MEFs that were infected with a retrovirus expressing the oncogenic H-Ras^{V12} allele; Sage et al., 2000; FIG. 2B) placed in suspension following trypsinization.

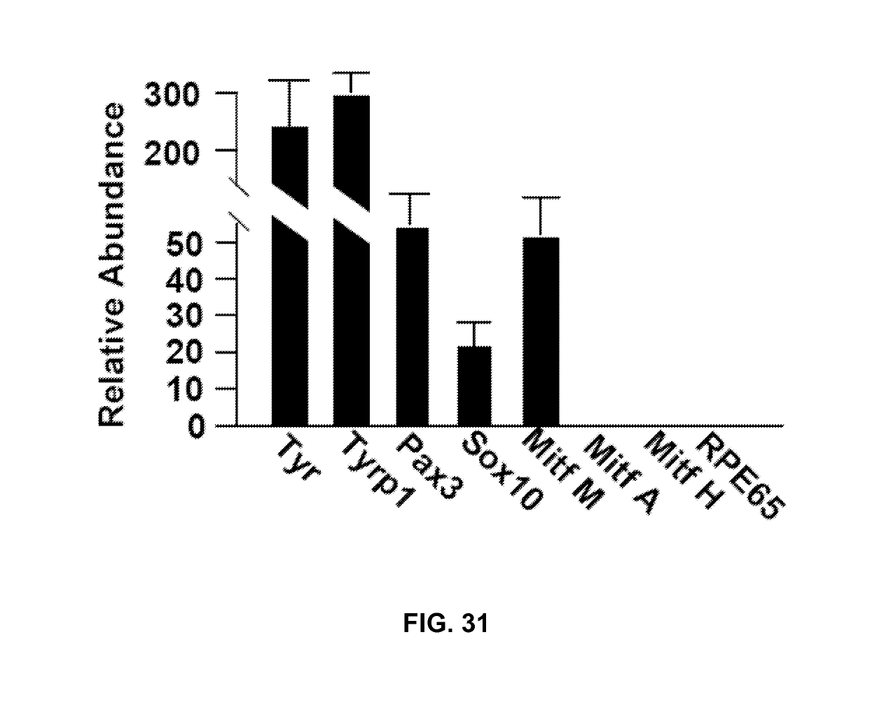

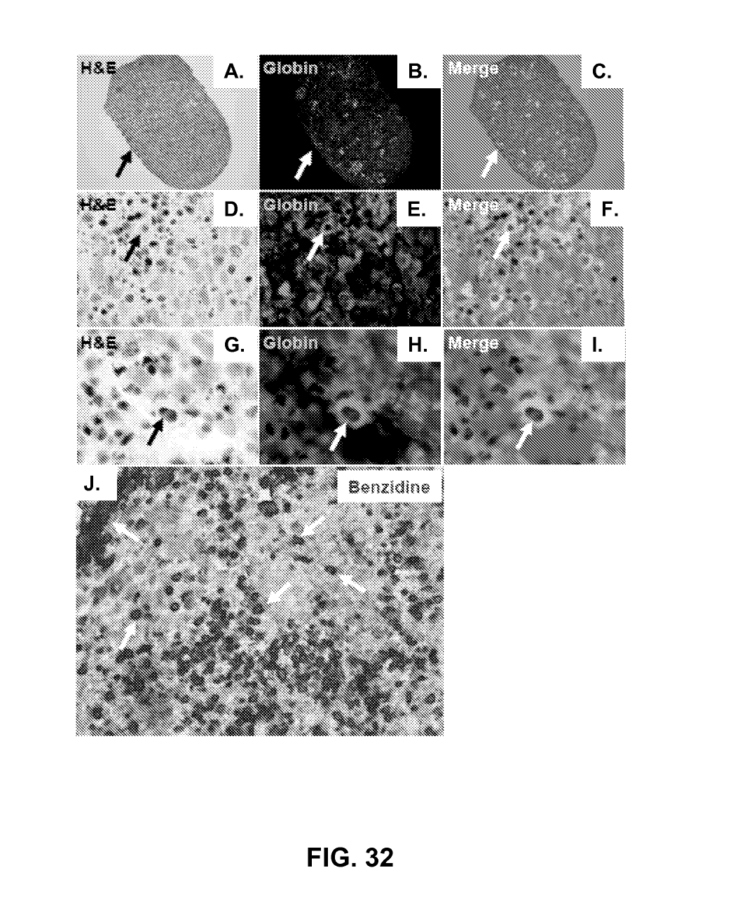

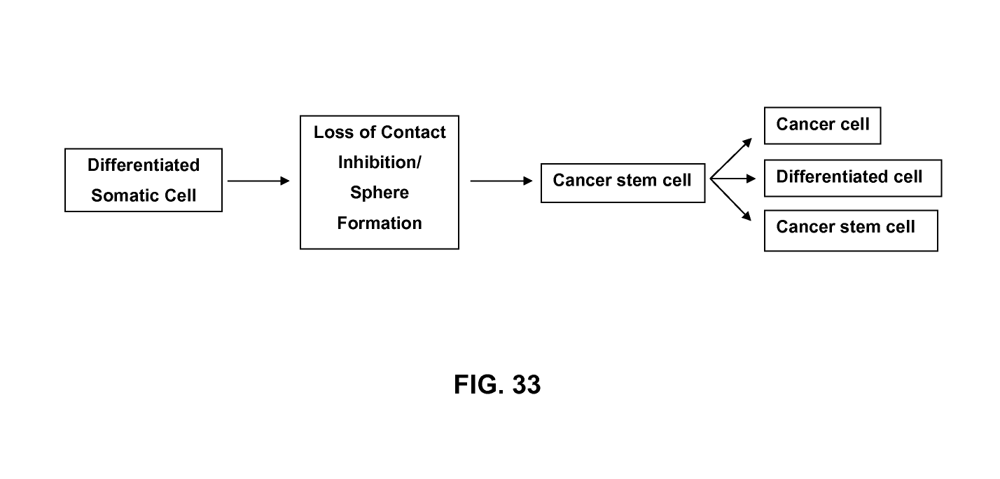

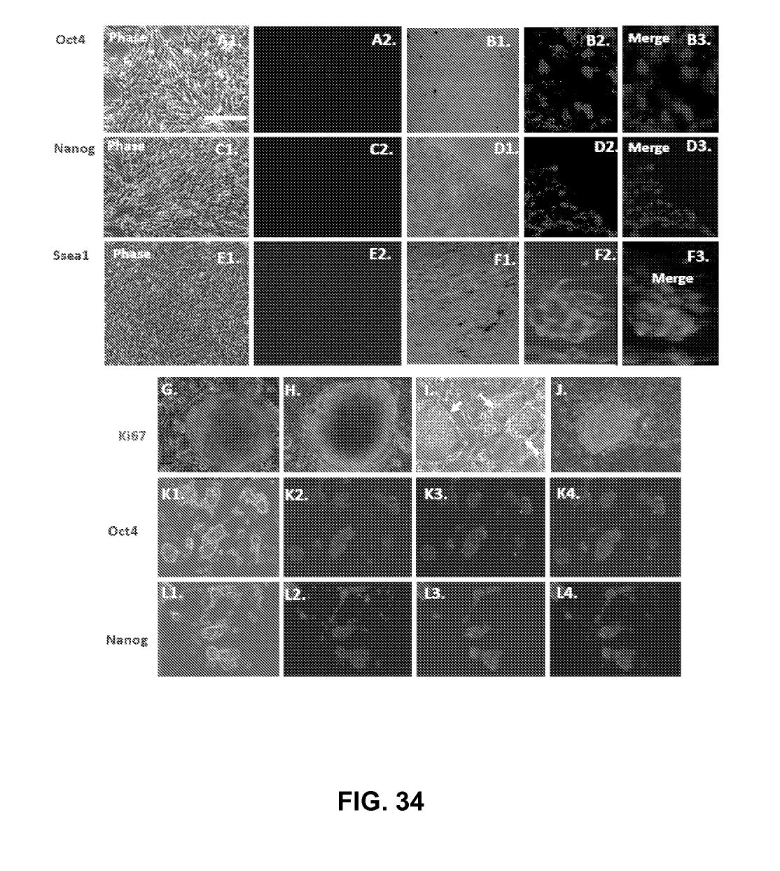

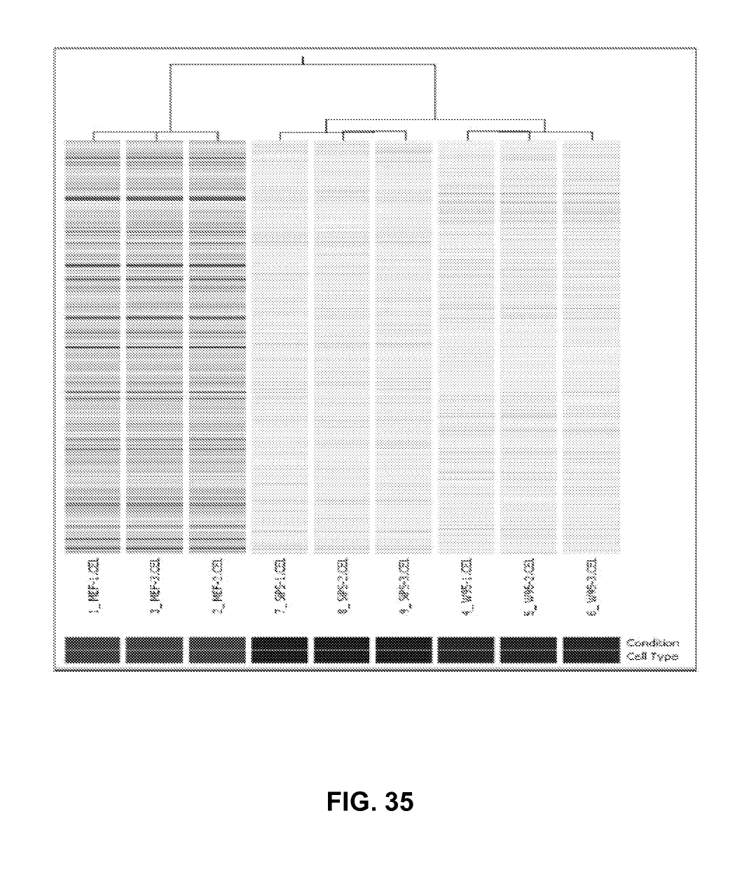

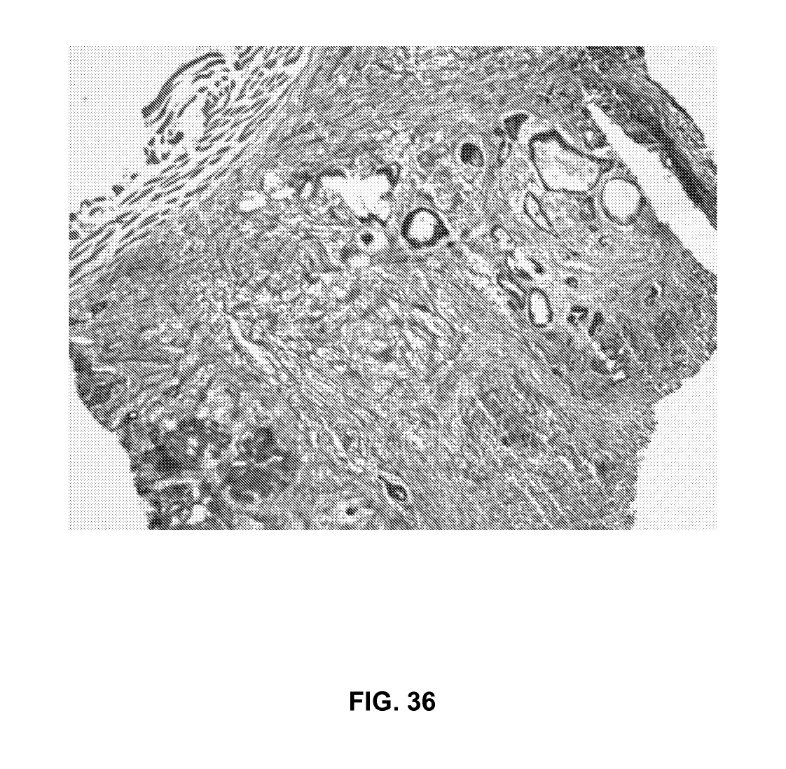

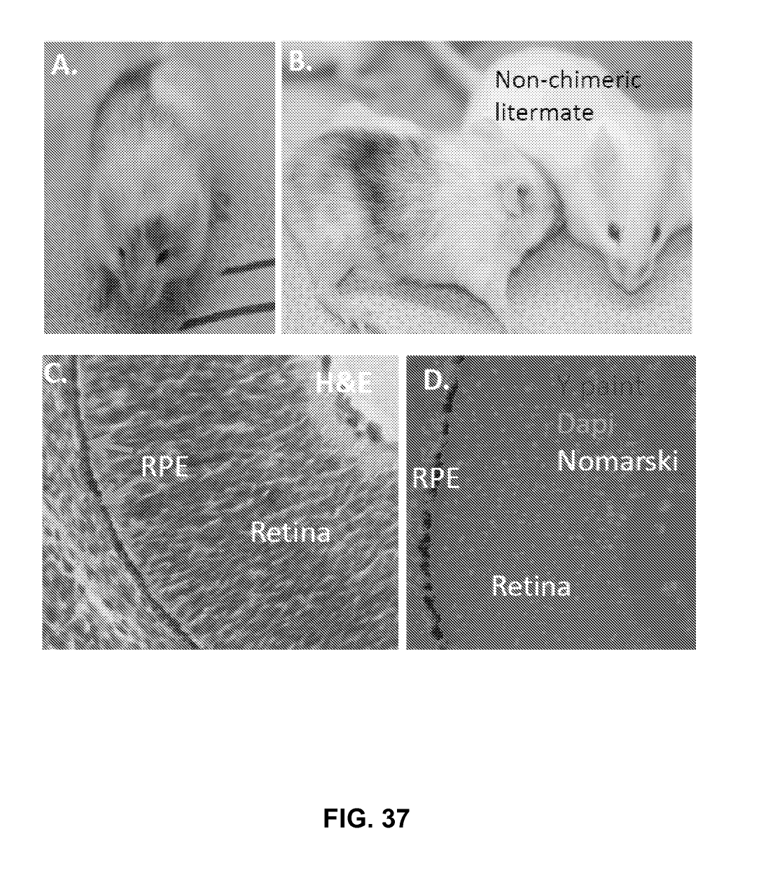

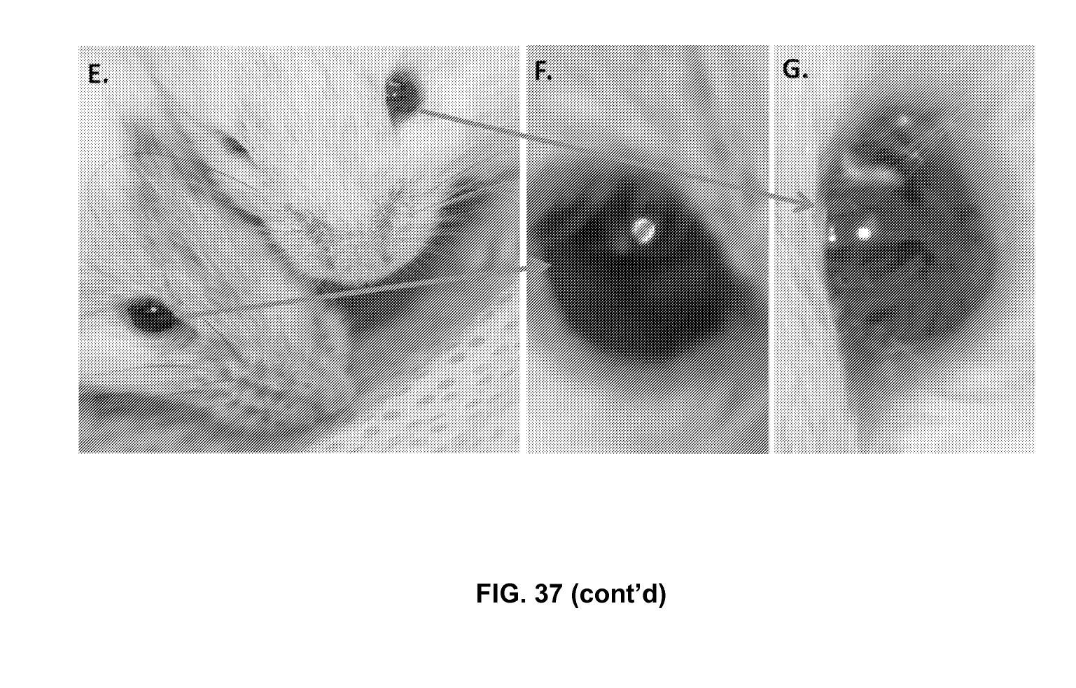

As shown in FIG. 2A, TKO cells did not form spheres in suspension. The cells died after 24 hours. Similar results were seen with RB1^{-/-} murine embryonic fibroblasts (MEFs). FIG. 2B shows that TKO-Ras cells also did not form spheres in suspension culture. Like TKO cells, TKO-Ras were not contact inhibited, but they detached from culture dishes as they became confluent and formed small groups or clusters of cells that survived in suspension and proliferated. These small groups or clusters of cells were distinguishable from the spheres of the presently disclosed subject matter in that individual cell borders remained visible and the cells were not tightly packed into a spherical structure with a defined border.

FIG. 3 is a series of bar graphs and photographs depicting the results of soft agar assays of TKOs, TKO cells derived from spheres (TKO Sphere), and TKO cells that overexpress Ras (TKO-Ras). Two independent assays are shown. Equal numbers of cells were plated, and visible colonies were counted after 3 weeks. Colony size was similar with TKO cells derived from spheres and TKO-Ras. Colonies formed with TKO cells were very small. The bar graphs below each photograph show the number of colonies per plate in each independent assay.

FIGS. 4A and 4B show Western blot analyses of Ras expression and activity in MEFs, TKOs, and TKO-Ras cells. To produce TKO-Ras cells, TKOs were infected with a H-Ras^{V12}-expressing retrovirus as described in Telang et al., 2006.

FIG. 4A is a digital image of a Western blot showing total Ras expression in TKOs and in TKO-Ras cells. The bottom panel of FIG. 4A shows β-actin expression, which was included as a loading control. FIG. 4B is a digital image of a Western blot showing activated Ras that was detected by binding to a fusion protein of Raf fused to glutathione-S-transferase (GST-Raf). The bottom panel of FIG. 4B shows a Western blot of input total Ras protein used for each assay. Note that not only did TKO-Ras cells have an increased level of Ras relative to TKOs (FIG. 4A), an increased percentage of the Ras present was in an activated form (FIG. 4B).

FIGS. 5A-5D are a series of photographs showing sphere formation in RB1^{-/-} MEFs led to stable morphological changes.

FIG. 5A shows RB1^{-/-} MEFs in monolayer culture. FIG. 5B shows spheres formed when cells were scraped from dishes and placed in suspension culture. FIG. 5C shows re-adhesion of an RB1^{-/-} MEF sphere to a tissue culture plate. Note that cells migrated from the sphere to reform a monolayer. FIG. 5D shows a higher power view of the cells in the box in FIG. 5C. Cells in FIGS. 5A and 5D are similar magnification and exemplify the differences in size and morphology of RB1^{-/-} MEFs in monolayer culture prior to (FIG. 5A) subsequent to (FIG. 5D) sphere formation.

FIGS. 6A-6D provide the results of experiments showing that sphere formation led to the expression of several stem cell markers in TKO and RB1^{-/-} MEF spheres, and to downregulation of RB1 family members (RB1, RBL1, and RBL2) in RB1^{-/-} MEFs.

FIG. 6A is a bar graph depicting the results of Real Time PCR assays showing induction of mRNAs for stem cell markers in TKO and RB1^{-/-} spheres after two weeks in suspension culture. Similar mRNA induction was maintained in monolayers derived from the spheres. FIG. 6B is a bar graph depicting the results of assays showing that Oct4 and Nanog mRNA increased in RB1^{-/-} spheres with the number of days (d) in culture. Real Time PCR was used to analyze mRNA levels. FIG. 6C is a series of photomicrographs showing the results of immunostaining for Oct4 in sections of RB1^{-/-} MEFs after 4 and 24 days in culture. The right hand panel of each 24 day photomicrograph depict a higher power view. Note only cytoplasmic staining at 4 days, whereas nuclear staining is evident at 24 days. No staining was evident in the absence of the Oct4 primary antibody. FIG. 6D is a bar graph providing the results of Real Time PCR demonstrating changes in expression of other mRNAs associated with stem cells and cancer stem cells after two weeks in suspension culture (see also FIG. 7). The comparison with respect to relative abundance is to expression of the listed genes in cells that are growing in subconfluent monolayers.

FIG. 7 is a bar graph showing the results of Real Time PCR analysis of mRNA levels of the listed genes in RB1^{-/-} cells after 8 days as spheres in suspension culture compared to RB1^{-/-} cells maintained as monolayers.

FIGS. 8A-8D show that sphere formation in TKOs or RB1^{-/-} MEFs generated cells with characteristics of a tumor side population (SP). Immunostaining for Abcg2 and CD133 is shown on the left, and Hoechst dye staining is shown on the right.

FIG. 8A is a series of fluorescence micrographs showing TKOs in subconfluent monolayer culture. FIG. 8B is a series of fluorescence micrographs showing cells derived from TKO spheres after two weeks in suspension culture. Similar results were seen with cells derived from RB1^{-/-} MEF spheres. FIG. 8C is a bar graph showing quantification of SP (Hoechst⁻/Abcg2⁺/CD133⁺) cells. FIG. 8D is a bar graph showing TKO and RB1^{-/-} MEF sphere-derived cells separated into SP (Hoechst⁻/Abcg2⁺/CD133⁺) and main population (MP; Hoechst⁺/Abcg2⁻/CD133⁻) and placed in culture (day 0). At the indicated times, the cells were again examined to quantify the appearance of MP cells within the SP population, and SP cells within the MP population.

FIG. 9 is a series of fluorescence micrographs of wild type MEFs and TKO cells maintained as subconfluent monolayers showing that these cells did not express CD133 or Abcg2 (left panels) or exclude Hoechst dye (right panels).

FIG. 10 is a FACS plot of TKO cells derived from spheres stained with Hoechst 33342 and propidium iodide (PI) dyes followed by analysis and sorting using a MOFLO™ cell sorter. Living cells were visualized on dot-plots according to their Hoechst red (Ho Red) and Hoechst blue (Ho Blue) fluorescences. SP cells excluding Hoechst 33342 were sorted from region R2 and the region enclosing only living cells identified based on PI staining (region R1, not shown). The percentage represented the content of SP in total sorted cells. Gates were set stringently to ensure no contamination with MP cells. Assessment of sorted SP cells revealed 100% Hoechst⁻/Abcg2⁺/CD133⁺ cells.

FIG. 11 is a bar graph showing about 50,000 sorted MP (Hoechst^{|}/Abcg2⁻/CD133⁻) and SP (Hoechst⁻/Abcg2⁺/CD133⁺) cells derived from spheres after two weeks in suspension culture placed in culture at day 0. SP and MP cells were then counted in the two populations after 3 days in culture. Note that SP cell number remained constant in the sorted SP cells, while this population gave rise to MP cells. Also note that sorted MP cells gave rise to a small population of SP cells (˜1%) by day three in culture.

FIGS. 12A-12E are a series of bar graphs showing the results of Real Time PCR analyses of SP cells with respect to various stem cell markers, and also showing that SP cells overexpressed the epithelial-mesenchymal transition (EMT) transcription factor Zeb1 and had a CD44^{high}/CD24^{low} mRNA pattern.

In FIG. 12A, TKO sphere-derived cells were separated into SP (Hoechst⁻/Abcg2⁻/CD133⁺) and MP (Hoechst⁺/Abcg2⁻/CD133⁻) by cell sorting, and Real Time PCR was used to assess the relative abundances of mRNAs or stem cell markers in these populations as compared to expression levels of these same markers in wild type W95 ES cells maintained in monolayer culture in the presence of LIF. Results shown are normalized to β-actin (ACTB) mRNA, but similar results were seen with normalization to glyceraldehyde 3-phosphate dehydrogenase (Gapdh) mRNA or β₂-microglobulin mRNA. FIG. 12B is a bar graph showing that Zeb1, but not Zeb2, Snai1, or Snai2 mRNA was induced in SP cells compared to the MP or unsorted sphere-derived cells. FIG. 12C is a bar graph showing that Zeb1 mRNA was induced in a time course during culture of RB1^{-/-} MEFs in suspension. FIG. 12D is a bar graph showing that CD44 mRNA was induced in SP cells, whereas CD24 was diminished. FIG. 12E is a bar graph showing that knockdown of Zeb1 (Zeb1 sh; an shRNA comprising SEQ ID NO: 72) but not Zeb2 (Zeb2 sh; an shRNA comprising SEQ ID NO: 73) induced expression of CD24 mRNA. Lentiviral shRNA constructs Zeb1 sh and Zeb2 sh were used to infect MEFs and efficiently knocked down Zeb1 and Zeb2 expression (see FIG. 13).

FIGS. 13A-13E show the results of lentiviral vector expression of green fluorescent protein (GFP) and shRNAs directed against Zeb1 or Zeb2 used to infect MEFs. Infection efficiency was >80%.

FIG. 13A is a set of photomicrographs showing an example of GFP expression in MEFs infected with a GFP-expressing lentiviral vector. FIGS. 13B and 13C are bar graphs showing RNA levels of Zeb1 and Zeb2 in uninfected vs. shRNA-containing cells, respectively, determined by Real Time PCR. FIGS. 13D and 13E are digital images of Western blots. shRNA sequences for mouse Zeb1 and Zeb2 knockdown are described in Nishimura et al., 2006 and in the Method and Materials for the EXAMPLES section herein below.

FIGS. 14A-14D are a series of photomicrographs showing TKO cells formed spheres when cultured in non-adherent culture plates.

FIG. 14A shows that 2 weeks after placing the cells in suspension, spheres began to form central cavities (denoted by the arrow). FIGS. 14B-14D show that the spheres aggregated into larger structures. Such structures are shown after 2 months in culture. FIGS. 14C and 14D are hematoxylin and eosin (H&E)-stained sections of the boxed region in FIGS. 14B and 14C, respectively.

FIGS. 15A-15I are a series of photomicrographs of H&E-stained sections of TKO spheres and aggregates after 3 weeks in non-adherent culture plates. Diverse cell morphologies are shown in the photomicrographs.

FIG. 15A shows a low power view of spheres containing cells of varying morphologies merging to form a large spherical structure. FIGS. 15B and 15C show cells with morphologies of hematopoietic cells. Cells were stained with H&E. The cells were very small cells with high nuclear to cytoplasmic ratio and intensely staining nuclei resembling lymphocytes. Additionally, the swirls of these cells resembled sites of hematopoietic differentiation typically seen in development.

FIGS. 15D-15I show cells with neural tissue morphologies. FIG. 15D shows H&E staining demonstrating cells with elongated projects resembling neurons. FIGS. 15E and 15F show cells with neuronal morphology and tissue resembling brain. FIGS. 15G-15I show additional cells with elongated morphology of neurons.

FIGS. 16A-16F are a series of bar graphs showing the results of Real Time PCR used to analyze the effect of sphere formation on expression of mRNAs representative of different embryonic layers (endoderm: FIG. 16A; ectoderm: FIG. 16B; and mesoderm: FIG. 16C), and the Wnt (FIG. 16D), Notch (FIG. 16E), and various growth factor (FIG. 16F) developmental signaling pathways. Relative mRNA expression in TKO subconfluent monolayers was compared to cells derived from TKO spheres which had been in suspension culture for three weeks. Similar results were seen with the spheres themselves. See FIG. 7 for similar analyses of RB1^{-/-} MEF spheres.

FIGS. 17A-17L are a series of photomicrographs of the results of immunostaining RB1^{-/-} spheres showing expression of markers representative of the three embryonic layers.

FIG. 17A is an H&E stained section of an RB1^{-/-} MEF sphere after two weeks in suspension culture. An arrow denotes the edge of the sphere. FIG. 17B is a higher power view of the perimeter of the sphere in FIG. 17A. Note the band of cells with endodermal-like morphology and eosinophilic cytoplasm. FIG. 17C is a higher power view of the region immediately interior to the band of cells at the perimeter of the sphere. Note cells with epithelial-like morphology. FIGS. 17D-17L show the results of immunostaining sections of RB1^{-/-} MEF spheres with antibodies directed against α-fetoprotein (AFP; FIGS. 17D and 17E), globin (FIGS. 17F-17H), CD31 (FIGS. 17I and 17J), E-cadherin (Cdh1; FIG. 17K), and β-III tubulin (Tubb3; FIG. 17L). Each of FIGS. 17D-17L includes a Nomarski image (panel 1), followed by immunostaining (panel 2), 4,6′-diamidino-2-phenylindole (DAPI) staining (panel 3), and a merged image (panel 4). Arrows denote the same position in each panel.

FIG. 18 is a series of photomicrographs of the results of immunostaining of 3 week old TKO spheres for representative markers of differentiation. α-fetoprotein (AFP); GATA4 (GATA); vimentin (Vim); α-tyrosine hydroxylase (αTH); β-III tubulin; myelin basic protein (MBP); 1s11; tyrosine hydroxylase (TH); and glial acidic fibrillary protein (GFAP). Wild type MEFs and TKOs prior to sphere formation did not immunostain for AFP, GATA4, TH, Is11, MBP, GFAP, or Tubb3. Wild type MEFs did express vimentin.

FIGS. 19A-19S are a series of photomicrographs of RB1^{-/-} MEF spheres after 24 days in suspension. FIGS. 19A-19O show autofluorescence in conjunction with H&E staining to allow assessment of cellular morphology. Note that most of the autofluorescent cells are nucleated. However, a subset of the cells lack nuclei (FIGS. 19N-19O). Cells in the perimeter of the spheres immunostained for globin (FIGS. 19M-19Q). Little green autofluorescence was seen in the absence of the primary globin antibody (FIGS. 19P-19Q). However, autofluorescence of the globin⁺ cells was seen with a red filter. This autofluorescence completely overlapped with globin immunostaining In addition to globin⁺ cells, H&E staining showed cells with characteristics of other hematopoietic cells (FIGS. 19R and 19S, the latter of which is a higher magnification of the boxed area shown in the former). Note the large multinucleated cell in the center resembling a megakaryocyte in FIGS. 19S and 19T.

FIGS. 20A-20L are a series of photographs and photomicrographs showing that SP cells are the primary tumorigenic population in the spheres, and tumors derived from these cells consist of cancer cells and neuronal whorls.

FIG. 20A is a photograph showing tumors formed in nude mice three weeks after injection of 100 SP cells subcutaneously into the hind leg. FIG. 20B is a photograph showing that tumors failed to form when 20,000 MP cells were similarly injected. FIGS. 20C and 20D are H&E stained sectioned tumors isolated from hind limbs of animals that were injected with 50,000 TKO-Ras cells (FIG. 20C) or 50,000 MP cells (FIG. 20D). These tumors were indistinguishable histologically, and appeared to be spindle cell sarcomas. Multiple tumors from the two cell types showed the same histology. FIG. 20E shows an H&E-stained section of a tumor formed three weeks after injection of 100 SP cells. Note the presence of numerous closely packed whorls with eosinophilic centers (arrows). FIG. 20F is a higher power view of a whorl (arrow) in the tumor from FIG. 20E. FIG. 20G shows a Nomarski image of a section of the tumor in FIG. 20E. FIG. 20H shows immunostaining of the section in FIG. 20G for β-III tubulin. Arrows in FIG. 20G and FIG. 20H indicate the same position. Only the whorls immunostained, and tumors derived form MP and TKO-Ras cells lacked these whorls and did not immunostain. FIG. 20I and FIG. 20J show nuclear immunostaining for Oct4 in a section of an SP cell-derived tumor. The boxed region in FIG. 20I is shown at higher magnification in FIG. 20J. FIG. 20K and FIG. 20L show nuclear immunostaining for Nanog in a section of an SP cell-derived tumor. FIG. 20L is a higher power view of the section shown in FIG. 20K.

FIGS. 21A-21D are a series of photomicrographs of tumors formed in nude mice.

FIG. 21A is an H&E-stained section of a tumor formed following injection of small spheres of TKO cells after two weeks in suspension culture into nude mice. Initially, 50,000 cells were employed for sphere formation. As a control, no tumors formed with 50,000 cells which were trypsinized and injected into nude mice as single cells. FIG. 21B is an H&E section of a tumor formed following injection of two week old RB1^{-/-} MEF spheres into nude mice. Note whorls with eosinophilic centers. FIG. 21C shows a Nomarski image of the tumor in FIG. 21B. FIG. 21D depicts immunostaining of FIG. 21C for β-III tubulin (Tubb3). Arrows in FIGS. 21C and 21D indicate the positions of whorls.

FIGS. 22A-22D depict analysis of spheres formed from wild type (i.e., RB1^{+/+}, RBL1^{|/|}, and RBL2^{|/|}) murine embryonic fibroblasts (MEFs).

FIG. 22A is a photomicrograph of spheres formed by wild type MEFs after one week in suspension culture, demonstrating that wild type fibroblasts can form spheres and survive in suspension culture. FIG. 22B is a bar graph showing the results of Real Time PCR analyses of the induction of mRNAs for genes associated with embryonic stem (ES) cells. Upregulation of the stem cell markers Oct4, Nanog, Klf4, Sox2, and SSEA1 was observed, suggested that MEFs present within the spheres were reprogrammed to an ES cell-like gene expression pattern by the techniques disclosed herein. Also, the mRNA for the epithelial-mesenchymal transition (EMT) transcription factor Zeb1 was induced. FIG. 22C is a series of photomicrographs of immunostaining of the spheres shown in FIG. 22A showing regions of cells expressing the stem cell markers Oct4, Nanog, and SSEA1. FIG. 22D is a bar graph of Real Time PCR analyses showing expression of mRNAs for a variety of transcription factors that drive differentiation as well as markers of differentiation of cell types from all three embryonic layers. mRNA expression was examined in spheres of wild type MEFs after one week in suspension culture.

FIGS. 23A-23P are photomicrographs of spheres formed from human foreskin fibroblasts (FIGS. 23A-23G) or wild type MEFs (FIGS. 23H-23P) after 2 weeks in culture.

FIG. 23A is a photomicrograph of endodermal-like cells at the border of the sphere after H&E staining FIGS. 23B and 23C show H&E staining of cells resembling nucleated blood cells. FIG. 23D shows benzidine staining, which demonstrated the presence of hemoglobin in many of the putative blood cells. FIGS. 23E-23G show the results of immunostaining the field shows in FIG. 23A for the endodermal marker α-fetoprotein (AFP; see FIG. 23E), the endothelial marker CD31 (see FIG. 23F), and α-globin (see FIG. 23G). Each of FIGS. 23E-23G includes five panels: Nomarski images (panel 1), DAPI staining (panel 2), immunostaining for the indicated genes (panel 3), merges of panels 2 and 3 (panel 4), and merges of panels 1-3 (panel 5). FIGS. 23H and 23I show low and high power views of H&E stained sections showing endothelial cells (white arrow in FIG. 23I) surrounding a blood vessel. A ductal structure is shown by the black arrow in FIG. 23I. FIG. 23J shows benzidine staining of wild type MEF spheres and demonstrates the presence of hemoglobin in the cells of these spheres. Panel 1 of FIG. 23K shows an H&E stain of an erythrocyte, and panel 2 of FIG. 23K shows immunostaining of an adjacent section of the sphere for globin, demonstrating that this erythrocyte expressed hemoglobin. FIG. 23L shows immunostaining of another erythrocyte for globin. This cell was nucleated as demonstrated by DAPI nuclear staining (panel 1), and was positive for globin (panel 2; panel 3 shows a merge of panels 1 and 2) demonstrating that wild type MEF spheres contained both nucleated and mature erythrocytes. FIG. 23M shows DAPI staining (panel 1); immunostaining for CD31, which is a marker of endothelial cells (panel 2); and a merge of panels 1 and 2 (panel 3); showing that endothelial cells are formed in the wild type MEF spheres. FIGS. 23N and 23O are photomicrographs showing a region of a wild type MEF-derived sphere containing cartilage, which is shown stained with alcian blue in FIG. 23O. FIG. 23P is a photomicrograph showing pearls of keratin (dark staining) in a keratinized cyst present within a wild type MEF-derived sphere.

FIGS. 24A-24F are photomicrographs of wild type MEFs allowed to form spheres in suspension culture for 3 weeks, demonstrating that these cells gave rise to differentiated structures and tissues.

FIG. 24A is a photomicrograph showing a secretory epithelium ascinar-like structure with a central duct (arrow). FIG. 24B is a photomicrograph showing secretory ducts (gray arrows) and red blood cells (white arrow). FIGS. 24C and 24D are photomicrographs showing immunostaining for the epithelial marker E cadherin (Cdh1) and the neuronal marker β-III tubulin (β3Tub). Each of FIGS. 24C and 24D includes four panels: panel 1 is a photograph of Nowarski optics, panel 2 is a DAPI stain showing cellular nuclei, panel 3 is an immunostain with an antibody directed against E cadherin or β-III tubulin, and panel 4 is a merge of panels 2 and 3. FIGS. 24E and 24F (the latter an enlargement of the field in the box in FIG. 24E) show hair fibers at the border of the spheres (the border is identified by black arrows). FIGS. 24A, 24B, 24E, and 24F depict H&E-stained cells.

FIGS. 25A-25Q are a series of photomicrographs of spheres produced by Hoechst⁻/Abcg2⁺/CD133⁺ cells derived from wild type MEFs that express a Green Fluorescent Protein (GFP) transgene after 2 weeks in culture. The Hoechst⁻/Abcg2⁻/CD133⁺ cells were isolated by cell sorting and cultured on a feeder layer of irradiated fibroblasts. Hoechst⁻/Abcg2⁺/CD133⁺ cells are shown on feeder layers after one day (FIGS. 25A and 25B) and after one week in culture (FIG. 25C). Immunostaining for the indicated markers is shown after one week in monolayer culture in FIGS. 25D-25Q. Each of FIGS. 25D-25Q includes three panels: the left panels show Nomarski images, the center panels show immunostaining for the indicated markers of the same fields as shown in the Nomarski images as well as nuclear localization with DAPI, and the right panels show merges of the left and center panels for each Figure.

FIGS. 26A-26E are a series of photomicrographs of teratoma formation by Hoechst⁻/Abcg2⁺/CD133⁺ cells derived from spheres derived from wild type MEFs that express GFP after 2 weeks in suspension culture. Four independent preparations of 50,000 cells were injected into both hind limbs of nude mice. Tumors were observed in all 8 injections, and were tumors were collected after three weeks.

FIG. 26A is a Nomarski image of a teratoma. FIG. 26B is a higher power view of an adjacent section of the tumor shown in FIG. 26A stained with H&E. Note the variety of structures characteristic of a teratoma. FIG. 26C shows DAPI nuclear staining of the section presented in FIG. 26A. The MEFs were isolated from Actin-GFP mice and immunostaining for GFP in FIG. 26D, which shows that the tumor is GFP⁺ whereas surrounding host tissue is GFP⁻. FIG. 26E is a merge of FIGS. 26C and 26D.

FIGS. 27A-27H are a series of photomicrographs of teratomas formed with Hoechst⁻/Abcg2⁺/CD133⁺ cells derived from wild type MEF spheres showing cobblestone epithelial morphology and expressing the epithelial specification protein E-cadherin.

FIGS. 27A-27D are a series of low power views. A Nomarski image of the section is shown in FIG. 27A. DAPI nuclear staining is shown in FIG. 27B, and E-cadherin immunostaining on the surface of the cells is shown in FIG. 27C. A merge of FIGS. 27B and 27C is shown in FIG. 27D.

FIGS. 27E-27H are a series of higher power images. A Nomarski image is shown in FIG. 27E. DAPI nuclear staining is shown in FIG. 27F, and E-cadherin immunostaining on the surface of the cells is shown in FIG. 27G. A merge of FIGS. 27F and 27G is shown in FIG. 27H.

FIGS. 28A-28P are a series of photomicrographs showing the formation of differentiated tissues in teratomas produced from Hoechst⁻/Abcg2⁺/CD133⁺ cells isolated from spheres derived from wild type MEFs expressing GFP. Tumors were isolated 3 weeks after injection of 50,000 cells and sectioned for immunostaining.

FIG. 28A is a Nomarski image of adipose tissue present in a teratoma. FIG. 28B shows DAPI staining showing cell nuclei. FIG. 28C shows immunostaining for GFP demonstrating that the adipose tissue is derived from the injected Hoechst⁻/Abcg2/CD133^{|} cells. FIG. 28D is a merge of FIGS. 28B and 28C.

FIG. 28E is a Nomarski image of a neuronal structure in a teratoma. FIG. 28F shows DAPI nuclear staining of the section in FIG. 28D. FIG. 28G shows immunostaining of the section of FIG. 28E for β-III tubulin, showing a cluster of neurons within a neuronal structure in the teratoma. FIG. 28H is a merge of FIGS. 28F and 28G.

FIG. 28I is a Nomarski image of a region of intestinal-like epithelium in a teratoma. FIG. 28J shows DAPI nuclear staining of the section of FIG. 28I. FIG. 28K shows immunostaining for GFP, and shows that this intestinal-like structure is derived from injected Hoechst⁻/Abcg2^{'1}/CD133⁺ cells. FIG. 28L is a merge of FIGS. 28J and 28K.

FIG. 28M is a Nomarski image of a secretory epithelium-like structure in a teratoma. FIG. 28N shows DAPI nuclear staining in the structure of FIG. 28M. FIG. 28O shows GFP immunostaining and demonstrates that the structure in FIG. 28M is derived from the injected Hoechst⁻/Abcg2⁺/CD133⁺ cells. FIG. 28P shows the results of immunostaining for CDH1, which demonstrates that the structure shown is epithelial. These Figures show the presence of multiple differentiated tissues in the teratomas formed with Hoechst⁻/Abcg2⁺/CD 133⁺ cells derived from wild type MEF cells that express a GFP transgene following sphere formation.

FIGS. 29A-29I are a series of photomicrographs showing formation of skeletal muscle in a teratoma arising from injection of Hoechst⁻/Abcg2⁺/CD133⁺ cells derived from spheres produced from wild type MEF expressing GFP into nude mice.

FIG. 29A is a photomicrograph of an H&E stained section showing skeletal muscle fibers in the teratoma. A Nomarski image of an adjacent section is shown as FIG. 29B. DAPI nuclear staining is shown in FIG. 29C, and GFP staining is shown in FIG. 29D, demonstrating that the muscle cells were tumor-derived. A merge is shown in FIG. 29E. FIGS. 29F-29I are a series of control photomicrographs. A Nomarski image of host skeletal muscle is shown in FIG. 29F. DAPI staining is shown in FIG. 29G and GFP is shown in FIG. 29H. There was a lack of GFP staining in FIG. 29H, which is muscle present in the host, which does not express GFP.

FIGS. 30A-30K are a series of photomicrographs of wild type MEF-derived spheres after two weeks in suspension culture. Spheres attached to the culture plates and cells began to migrate out onto the culture plates as with TKO and RB1^{-/-} MEF spheres. However, in contrast to the TKO and RB1^{-/-} MEFs, only a portion of the cells from the wild type MEF spheres migrated back onto the plate. These cells were highly pigmented (see FIGS. 30A-30C). Initially, most of the cells were rounded or epithelial in appearance. However, after several days on the culture plates, the cells remained pigmented but began to elongate (see FIGS. 30D-30F). FIGS. 30G and 30H show lower power views of the cells.

FIGS. 30I-30K each consist of five panels: panel 1 is Nomarski optics, panel 2 is DAPI staining to show cell nuclei, panel 3 is staining for Mitf (FIGS. 30I and 30J) or Mel5 (FIG. 30K), panel 4 is a merge of panels 2 and 3, and panel 5 is a merge of panels 1-3. FIGS. 30I and 30J show immunostaining of these cells for the melanocyte marker Mitf (FIG. 30J being a higher power magnification of FIG. 30I), and FIG. 30K shows immunostaining of the cells for a second melanocyte marker Mel5. Taken together, these results demonstrated that immature melanosomes were formed in the spheres (the highly pigmented cells lacking dendritic extensions in FIGS. 30A-30D), and when the spheres were allowed to attach to a culture plate, these cells migrated from the spheres onto the culture plate and underwent differentiation as characterized by dendrite formation and expression of two markers of melanocytes. Melanocyte differentiation is also a property shared by ES cells and iPSC.

FIG. 31 is a bar graph showing gene expression analysis of the cells shown in FIG. 30. The Real Time PCR results for mRNA levels were compared to monolayers of control wild type MEFs prior to sphere formation and expressed as Relative Abundance (i.e., a ratio of expression in MEF-derived spheres to expression in MEF-derived monolayers prior to sphere formation).

FIGS. 32A-32J are a series of photomicrographs showing primary cultures of human lung bronchial epithelial cells grown to confluence, scraped from tissue culture dishes, and placed in suspension culture in non-adherent plates as described herein for fibroblasts. Spheres were allowed to form for 5 days, and then the spheres were fixed and sectioned into 5 micron sections.

FIGS. 32A-32C show sections of an exemplary sphere stained with H&E (FIG. 32A), immunostained for the presence of globin (FIG. 32B), and a merge of the H&E and immunostained fields (FIG. 32C) demonstrating erythrocyte differentiation in the spheres.

FIGS. 32D-32I show higher power views of an exemplary sphere showing erythrocytes immunostaining for globin.

FIG. 32J shows benzidine staining of a section of an exemplary sphere, further demonstrating the presence of hemoglobin.

FIG. 33 depicts a proposed, non-limiting model of a pathway for generation of cells with properties of cancer stem cells from differentiated somatic cells.

FIGS. 34A-34L are a series of photomicrographs of mouse neonatal skin fibroblasts and cells derived there from at various stages of induction to form sphere-induced pluripotent cells (siPS).

FIGS. 34A, 34C, and 34E are photomicrographs of neonatal skin fibroblasts immunostained with antibodies against Oct4, Nanog, and Ssea1, respectively. For each of FIGS. 34A, 34C, and 34E, panel 1 is a bright field image of fibroblasts prior to sphere formation and panel 2 is the panel 1 cells immunostained with the appropriate antibody. The absence of staining in panel 2 of each figure is indicative of a lack of expression of these markers in fibroblasts prior to sphere formation.

FIGS. 34B, 34D, and 34F are photomicrographs of neonatal skin fibroblast-derived cells immunostained with antibodies against Oct4, Nanog, and Ssea1, respectively, after the cells had formed spheres and been replated on feeder layers. For each of FIGS. 34B, 34D, and 34F, panel 1 is a low power photomicrograph of sphere-derived cells stained with the appropriate antibody, panel 2 is a high power photomicrograph of the sphere-derived cells in panel 1, and panel 3 is a merge of the panel 2 cells immunostained with the appropriate antibody and stained with the nuclear stain DAPI.

FIG. 34G is a bright field photomicrograph of a sphere of mouse tail fibroblast sphere-derived cells after 7 days in suspension culture immediately after re-plating on irradiated fibroblasts. FIG. 34H is a bright field photomicrograph of the same mouse tail fibroblast sphere-derived cells shown in FIG. 34H one (1) day after growth in culture, showing the migration of cells out of the sphere. FIG. 34I is a bright field photomicrograph of embryonic stem (ES) cell-like colonies (indicated by arrows) which arose from the spheres of mouse tail fibroblast sphere-derived cells. Spheres were plated on the feeder layer, and after one week the cultures were trypsinized and replated onto new feeder layers. Two weeks later, ES cell-like colonies were evident. The arrows indicate colonies that have the distinctive morphology typical of mouse ES cell colonies growing on fibroblasts.

FIG. 34J is a photomicrograph of the a colony like that in FIG. 34I immunostained for Ki67, which is a marker of cell proliferation, thus demonstrating that the cells in the colonies were dividing.

FIGS. 34K and 34L are a series of photomicrographs of sphere-derived cells immunostained for Oct4 and Nanog, respectively, demonstrating that the cells in the colonies expressed these stem cell factors in a manner reminiscent of embryonic stem cells. In each of FIGS. 34K and 34L, panels 1-4 are bright field, DAPI staining, anti-Oct 4 or anti-Nanog staining, and a merge of panels 3 and 4, respectively.

FIG. 35 is a heat map of gene expression patters of murine embryonic fibroblasts (MEF), sphere-induced pluripotent cells (siPS), and wild type murine ES cells (W95). Each cell type was tested in triplicate, thereby resulting in 3 heat maps per cell type.

FIG. 36 is a photomicrograph of a tumor formed three weeks after transplanting 50,000 siPS into the hind limbs of nude mice. Frozen sections of recovered tumors were stained with H&E. Histological analysis of the tumors indicated that the tumors were teratomas as tissues representative of all three embryonic layers were present.

FIGS. 37A-37G are a series of photographs of chimeric mice (or specific tissues thereof) generated by introducing siPS derived from male C57BL/6-derived MEFs into albino host mouse blastocysts and transferring the host blastocyst to pseudopregnant female mice, where they developed to term and were born. Hence, the chimeric animals shown in FIGS. 37A-37G exhibited coat and eye color chimerisms indicative of the contribution of the siPS to the epidermal layer of the chimeras.

FIG. 37A is a photograph of an exemplary chimeric mouse generated from C57BL/6-derived MEFs. Note the black hairs present, which are indicative of the contribution of the C57BL/6-derived MEFs to the epidermis of the chimera. This chimera also has eyes that are considerably darker than those seen in albino animals, indicative of the contribution of the C57BL/6-derived MEFs to the retinal pigmented epithelium (RPE) of the chimera.

FIG. 37B is a photograph of an exemplary chimeric mouse (left) and a non-chimeric littermate (right). Non-chimeric animals had white fur and red eyes, consistent with their albino phenotype.

FIGS. 37C and 37D are photomicrographs of sections through the eyes of anatomically female chimeric embryos at embryonic day 15 (E15) of development. In FIG. 37C, hematoxylin and eosin (H&E) staining of the section was employed to show the cellular structure of the tissues in the section. In particular, FIG. 37C shows a dark-staining RPE, which demonstrated the contribution of the C57BL/6-derived MEFs to the RPE of the chimera. FIG. 37D is a fluorescence micrograph of the same region of the eye using Nomarksi optics. The lighter gray areas were observed to be stained blue with DAPI when the field was viewed in color, which shows the locations of cellular nuclei. The light stippling when the field was viewed in color was pink staining (Y paint) that was specific for cells that have a Y chromosome (i.e., cells that are derived from the siPS generated from male C57BL/6-derived MEFs), thereby demonstrating the extensive contribution of the siPS to the eye of the chimera.

FIGS. 37E-37G are close up photographs of the eyes of exemplary chimeric animals, with the darker regions showing varying extents of siPS contributions to the eyes of these chimeras.

### BRIEF DESCRIPTION OF THE SEQUENCE LISTING

SEQ ID NOs: 1-70 are the nucleotide sequences of oligonucleotide primers that can be employed in pairwise combination (e.g., SEQ ID NOs: 1 and 2; SEQ ID NOs: 3 and 4, SEQ ID NOs: 5 and 6, etc.) to detect the expression of the 25 genes listed in Table 1 below.

SEQ ID NO: 71 is the nucleotide sequence of an oligonucleotide that specifically binds to an SP6 promoter fragment.

SEQ ID NO: 72 is a nucleotide sequence of an exemplary shRNA sense strand that can be used to knockdown expression of Zeb1.

SEQ ID NO: 73 is a nucleotide sequence of an exemplary shRNA sense strand that can be used to knockdown expression of Zeb2.

SEQ ID NO: 74 is a nucleotide sequence of a control shRNA sense strand that can be used to test the specificity of the shRNAs comprising SEQ ID NO: 72 or SEQ ID NO: 73 used to knockdown expression of Zeb1 or Zeb2, respectively.

TABLE 1
Summary of PCR Primers Employed

| | | | Tₘ | Ampl. |
|---|---|---|---|---|
| Gene | Primer Pair Sequences | | (° C.) | Size |
| Aldob | AGTGGCGTGCTGTGTTGAG | (SEQ ID NO: 1) | 61 | 122 |
| | AACAATAGGGACCAGCCCATT | (SEQ ID NO: 2) | 62 | bp |
| Acta2 | GTCCCAGACATCAGGGAGTAA | (SEQ ID NO: 3) | 59 | 102 |
| | TCGGATACTTCAGCGTCAGGA | (SEQ ID NO: 4) | 63 | bp |
| Des | GTGGATGCAGCCACTCTAG | (SEQ ID NO: 5) | 57 | 218 |
| | TTAGCCGCGATGGTCTCATA | (SEQ ID NO: 6) | 62 | bp |
| CD34 | AAGGCTGGGTGAAGACCCTTA | (SEQ ID NO: 7) | 62 | 157 |
| | TGAATGGCCGTTTCTGGAAGT | (SEQ ID NO: 8) | 64 | bp |
| Col4 | CAAGCATAGTGGTCCGAGTC | (SEQ ID NO: 9) | 58 | 463 |
| | AGGCAGGTCAAGTTCTAGCG | (SEQ ID NO: 10) | 60 | bp |
| GATA4 | CACCCCAATCTCGATATGTTT | (SEQ ID NO: 11) | 59 | 151 |
| | GGTTGATGCCGTTCATCTTGT | (SEQ ID NO: 12) | 62 | bp |
| Myh2 | AAGTGACTGTGAAAACAGAA | (SEQ ID NO: 13) | 51 | 222 |
| | GCAGCCATTTGTAAGGGTTGA | (SEQ ID NO: 14) | 62 | bp |
| LAMB- | GAAAGGAAGACCCGAAGAAA | (SEQ ID NO: 15) | 58 | 131 |
| 1 | CCATAGGGCTAGGACACCAAA | (SEQ ID NO: 16) | 61 | bp |
| Nes | AACTGGCACACCTCAAGATGT | (SEQ ID NO: 17) | 56.8 | 235 |
| | TCAAGGGTATTAGGCAAGGGG | (SEQ ID NO: 18) | 56.5 | bp |
| Trf | TCCTCCACTCAACCATTCTT | (SEQ ID NO: 19) | 57 | 149 |
| | TCAAGGCAGAGCAGTTCATA | (SEQ ID NO: 20) | 57 | bp |
| FGFR2 | GGATCTTCATGGTGAATGTCA | (SEQ ID NO: 21) | 58 | 103 |
| | CTCTGGTTGCTCCTGTTCTCA | (SEQ ID NO: 22) | 61 | bp |
| BMP4 | GACTTCGAGGCGACACTTCTA | (SEQ ID NO: 23) | 60 | 267 |
| | GTTGAAGAGGAAACGAAAAGCA | (SEQ ID NO: 24) | 61 | bp |
| FGF9 | TCTTCCCCAACGGTACTATC | (SEQ ID NO: 25) | 57 | 124 |
| | CCGAGGTAGAGTCCACTGT | (SEQ ID NO: 26) | 55 | bp |
| Oct4 | AGTTGGCGTGGAGACTTTGC | (SEQ ID NO: 27) | 58.2 | 160 |
| | CAGGGCTTTCATGTCCTGG | (SEQ ID NO: 28) | 56 | bp |
| Prom1 | GTTGAGACTGTGCCCATGAAA | (SEQ ID NO: 29) | 55.5 | 98 |
| | GACGGGCTTGTCATAACAGGA | (SEQ ID NO: 30) | 57 | bp |
| Msi1 | CCTCTCACGGCTTATGGGC | (SEQ ID NO: 31) | 58.1 | 271 |
| | CTGTGGCAATCAAGGGACC | (SEQ ID NO: 32) | 56.2 | bp |
| CD44 | TCTGCCATCTAGCACTAAGAGC | (SEQ ID NO: 33) | 56.3 | 106 |
| | GTCTGGGTATTGAAAGGTGTAGC | (SEQ ID NO: 34) | 55.4 | bp |
| CD24a | ACCCACGCAGATTTACTGCAA | (SEQ ID NO: 35) | 57.2 | 101 |
| | CCCCTCTGGTGGTAGCGTTA | (SEQ ID NO: 36) | 58.7 | bp |
| Flot2 | TGTGAGGACGTAGAGACGG | (SEQ ID NO: 37) | 55.8 | 148 |
| | GCAGCACGACGTTCTTAATGTC | (SEQ ID NO: 38) | 56.5 | bp |
| Nanog | TTGCTTACAAGGGTCTGCTACT | (SEQ ID NO: 39) | 56 | 106 |
| | ACTGGTAGAAGAATCAGGGCT | (SEQ ID NO: 40) | 55.4 | bp |
| Sox2 | GCGGAGTGGAAACTTTTGTCC | (SEQ ID NO: 41) | 56.7 | 157 |
| | CGGGAAGCGTGTACTTATCCTT | (SEQ ID NO: 42) | 56.7 | bp |
| Stat3 | AGCTGGACACACGCTACCT | (SEQ ID NO: 43) | 58.7 | 190 |
| | AGGAATCGGCTATATTGCTGGT | (SEQ ID NO: 44) | 56 | bp |
| Sca1 | AGGAGGCAGCAGTTATTGTGG | (SEQ ID NO: 45) | 57.4 | 114 |
| | CGTTGACCTTAGTACCCAGGA | (SEQ ID NO: 46) | 55.9 | bp |
| ACTB | GGCTGTATTCCCCTCCATCG | (SEQ ID NO: 47) | 57.6 | 154 |
| | CCAGTTGGTAACAATGCCATGT | (SEQ ID NO: 48) | 55.9 | bp |
| GAPDH | AGGTCGGTGTGAACGGATTTG | (SEQ ID NO: 49) | 57.6 | 123 |
| | TGTAGACCATGTAGTTGAGGTCA | (SEQ ID NO: 50) | 55.1 | bp |
| Pax3 | GGGCAGAATTACCCACGCA | (SEQ ID NO: 51) | 58.1 | 154 |
| | CTGGCGAGAAATGACGCAA | (SEQ ID NO: 52) | 55.9 | bp |
| Sox10 | ACACCTTGGGACACGGTTTTC | (SEQ ID NO: 53) | 57.9 | 123 |
| | TAGGTCTTGTTCCTCGGCCAT | (SEQ ID NO: 54) | 58.1 | bp |
| Tyr | AGTCGTATCTGGCCATGGCTTCTT | (SEQ ID NO: 55) | 60.3 | 145 |
| | ACAGCAAGCTGTGGTAGTCGTCTT | (SEQ ID NO: 56) | 60.4 | bp |
| Tyrp1 | ATACTGGGACCAGATGGCAACACA | (SEQ ID NO: 57) | 60.3 | 137 |
| | AAGCGGGTCCTTCGTGAGAGAAAT | (SEQ ID NO: 58) | 60.3 | bp |
| RPE65 | TGGATCTCTGTTGCTGGAAAGGGT | (SEQ ID NO: 59) | 60.3 | 177 |
| | AGGCTGAGGAGCCTTCATAGCATT | (SEQ ID NO: 60) | 60.2 | bp |
| MITF | TTGATGGATCCGGCCTTGCAAATG | (SEQ ID NO: 61) | 60.3 | 165 |
| | TATGTTGGGAAGGTTGGCTGGACA | (SEQ ID NO: 62) | 60.5 | bp |
| MITF-A | TTCACGAAGAACCCAAAACC | (SEQ ID NO: 63) | 53.3 | 135 |
| | AGTTGCTGGCGTAGCAAGAT | (SEQ ID NO: 64) | 57.1 | bp |
| MITF-H | GATGGAGGCGCTTAGATTTGA | (SEQ ID NO: 65) | 54.9 | 139 |
| | CATGAGTTGCTGGCGTAGCA | (SEQ ID NO: 66) | 58 | bp |
| MITF- | GCTGGAAATGCTAGAATAC | (SEQ ID NO: 67) | 48.1 | 172 |
| M | GGCTGGCATGTTTATTTGCT | (SEQ ID NO: 68) | 54.2 | bp |
| ACTB | GGCTGTATTCCCCTCCATCG | (SEQ ID NO: 69) | 57.6 | 154 |
| | CCAGTTGGTAACAATGCCATGT | (SEQ ID NO: 70) | 55.9 | bp |

### DETAILED DESCRIPTION

Disclosed herein is the discovery that outgrowth of fibroblasts in which all three retinoblastoma (RB1) family members have been mutated (referred to herein as “triple knockouts”; TKOs) into spheres led to stable reprogramming of the cells to a cancer stem cell phenotype. While fibroblasts containing only an RB1 mutation retained cell contact inhibition, bypassing this inhibition by forcing the cells to form spheres in suspension led to downregulation of RBL1 and RBL2, and to similar reprogramming of the RB1^{-/-} cells to a cancer stem cell phenotype. These cancer stem cells not only divided asymmetrically to produce cancer cells, they also generated differentiated cells. The results presented herein provide evidence of a potential pathway for generation of cancer stem cells from differentiated somatic cells. Based at least in part on these findings, disclosed herein is a new tumor suppressor function for the RB1 pathway that imposes contact inhibition to prevent outgrowth of differentiated somatic cells into spherical structures where reprogramming to cancer stem cells can occur.

Also disclosed herein is the discovery that when wild type mouse or human fibroblasts were induced to form spheres, they were also reprogrammed, but these cells only gave rise to differentiated cells; i.e., they did not produce cancer stem cells or cancer cells. Therefore, an intact RB1 pathway can prevent cancer cell formation when fibroblasts are reprogrammed by sphere formation.

Also disclosed herein is the discovery that when cells reprogrammed by the methods of the presently disclosed subject matter are reintroduced into embryos, they can contribute to some or all cell and tissue types in the developing embryo, thereby forming chimeric animals.

### I. Definitions

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

Following long-standing patent law convention, the terms “a”, “an”, and “the” mean “one or more” when used in this application, including the claims. Thus, the phrase “a stem cell” refers to one or more stem cells, unless the context clearly indicates otherwise.

Unless otherwise indicated, all numbers expressing quantities of ingredients, reaction conditions, and so forth used in the specification and claims are to be understood as being modified in all instances by the term “about”. The term “about”, as used herein when referring to a measurable value such as an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods. Accordingly, unless indicated to the contrary, the numerical parameters set forth in this specification and attached claims are approximations that can vary depending upon the desired properties sought to be obtained by the presently disclosed subject matter.

As used herein, the term “and/or” when used in the context of a list of entities, refers to the entities being present singly or in combination. Thus, for example, the phrase “A, B, C, and/or D” includes A, B, C, and D individually, but also includes any and all combinations and subcombinations of A, B, C, and D.

The term “comprising”, which is synonymous with “including” “containing”, or “characterized by”, is inclusive or open-ended and does not exclude additional, unrecited elements and/or method steps. “Comprising” is a term of art that means that the named elements and/or steps are present, but that other elements and/or steps can be added and still fall within the scope of the relevant subject matter.

As used herein, the phrase “consisting of” excludes any element, step, or ingredient not specifically recited. For example, when the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of the related disclosure or claim to the specified materials and/or steps, plus those that do not materially affect the basic and novel characteristic(s) of the disclosed and/or claimed subject matter. For example, a pharmaceutical composition can “consist essentially of” a pharmaceutically active agent or a plurality of pharmaceutically active agents, which means that the recited pharmaceutically active agent(s) is/are the only pharmaceutically active agent(s) present in the pharmaceutical composition. It is noted, however, that carriers, excipients, and other inactive agents can and likely would be present in the pharmaceutical composition.

With respect to the terms “comprising”, “consisting essentially of”, and “consisting of”, where one of these three terms is used herein, the presently disclosed and claimed subject matter can include the use of either of the other two terms. For example, the presently disclosed subject matter relates in some embodiments to compositions that comprise reprogrammed cells. It is understood that the presently disclosed subject matter thus also encompasses compositions that in some embodiments consist essentially of reprogrammed cells, as well as compositions that in some embodiments consist of reprogrammed cells. Similarly, it is also understood that in some embodiments the methods of the presently disclosed subject matter comprise the steps that are disclosed herein and/or that are recited in the claims, in some embodiments the methods of the presently disclosed subject matter consist essentially of the steps that are disclosed herein and/or that are recited in the claims, and in some embodiments the methods of the presently disclosed subject matter consist of the steps that are disclosed herein and/or that are recited in the claim.

The term “subject” as used herein refers to a member of any invertebrate or vertebrate species. Accordingly, the term “subject” is intended to encompass any member of the Kingdom Animalia including, but not limited to the phylum Chordata (i.e., members of Classes Osteichythyes (bony fish), Amphibia (amphibians), Reptilia (reptiles), Ayes (birds), and Mammalia (mammals)), and all Orders and Families encompassed therein.

Similarly, all genes, gene names, and gene products disclosed herein are intended to correspond to homologs and/or orthologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates. Thus, a given nucleic acid or amino acid sequence is intended to encompass homologous and/or orthologous genes and gene products from other animals including, but not limited to other mammals, fish, amphibians, reptiles, and birds.

The methods and compositions of the presently disclosed subject matter are particularly useful for warm-blooded vertebrates. Thus, the presently disclosed subject matter concerns mammals and birds. More particularly provided is the isolation, manipulation, and use of reprogrammed somatic cells from mammals such as humans and other primates, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), rodents (such as mice, rats, and rabbits), marsupials, and horses. Also provided is the use of the disclosed methods and compositions on birds, including those kinds of birds that are endangered, kept in zoos, as well as fowl, and more particularly domesticated fowl, e.g., poultry, such as turkeys, chickens, ducks, geese, guinea fowl, and the like, as they are also of economic importance to humans. Thus, also provided is the isolation, manipulation, and use of reprogrammed somatic cells from livestock, including but not limited to domesticated swine (pigs and hogs), ruminants, horses, poultry, and the like.

The term “isolated”, as used in the context of a nucleic acid or polypeptide (including, for example, a peptide), indicates that the nucleic acid or polypeptide exists apart from its native environment. An isolated nucleic acid or polypeptide can exist in a purified form or can exist in a non-native environment.

The terms “nucleic acid molecule” and “nucleic acid” refer to deoxyribonucleotides, ribonucleotides, and polymers thereof, in single-stranded or double-stranded form. Unless specifically limited, the term encompasses nucleic acids containing known analogues of natural nucleotides that have similar properties as the reference natural nucleic acid. The terms “nucleic acid molecule” and “nucleic acid” can also be used in place of “gene”, “cDNA”, and “mRNA”. Nucleic acids can be synthesized, or can be derived from any biological source, including any organism.

Several genes are disclosed herein. Representative sequences of nucleic acid and amino acid products from these genes are set forth in Table 2. It is understood that while Table 2 discloses Accession Numbers for certain of these genes that can be found in the GENBANK® database as they relate to humans and mice, other sequences from humans, mice, and other species are also included within the scope of the present disclosure and would be known and/or identifiable by one of ordinary skill in the art after consideration of the instant disclosure.

TABLE 2
GENBANK ® Accession Nos. for Representative Nucleic acid
and Amino acid Sequences

| Gene | | Homo sapiens | Mus musculus |
|---|---|---|---|
| β-III tubulin | Nucleic acid | NM_006086 | NM_023279 |
| | Amino acid | NP_006077 | NP_075768 |
| C-peptide | Nucleic acid | NM_000207^{a} | NM_008386^{b} |
| | Amino acid | NP_000198 | NP_032412 |
| FGF4 | Nucleic acid | NM_002007 | NM_010202 |
| | Amino acid | NP_001998 | NP_034332 |
| GATA4 | Nucleic acid | NM_002052 | NM_008092 |
| | Amino acid | NP_002043 | NP_032118 |
| GFAP | Nucleic acid | NM_002055 | NM_010277 |
| | Amino acid | NP_002046 | NP_034407 |
| KLF4 | Nucleic acid | NM_004235 | NM_010637 |
| | Amino acid | NP_004226 | NP_034767 |
| NANOG | Nucleic acid | NM_024865 | NM_028016 |
| | Amino acid | NP_079141 | NP_082292 |
| NESTIN | Nucleic acid | NM_006617 | NM_016701 |
| | Amino acid | NP_006608 | NP_057910 |
| NKX6-1 | Nucleic acid | NM_006168 | NM_144955 |
| | Amino acid | NP_006159 | NP_659204 |
| NKX2-5/CSX | Nucleic acid | NM_004387 | NP_004378 |
| | Amino acid | NM_008700 | NP_032726 |
| OCT4 | Nucleic acid | NM_002701 | NM_013633 |
| | Amino acid | NP_002692 | NP_038661 |
| OLIG1 | Nucleic acid | NM_138983 | NM_016968 |
| | Amino acid | NP_620450 | NP_058664 |
| OLIG2 | Nucleic acid | NM_005806 | NM_016967 |
| | Amino acid | NP_005797 | NP_058663 |
| PDX1 | Nucleic acid | NM_000209 | NM_008814 |
| | Amino acid | NP_000200 | NP_032840.1 |
| SOX2 | Nucleic acid | NM_003106 | NM_011443 |
| | Amino acid | NP_003097 | NP_035573 |
| SSEA1 | Nucleic acid | NM_002033 | NM_010242 |
| | Amino acid | NP_002024 | NP_034372 |
| STAT3 | Nucleic acid | NM_139276 | NM_213659 |
| | Amino acid | NP_644805 | NP_998824 |

^{a}NM_000207 is a nucleotide sequence of human insulin. Nucleotides 228-320 of NM_000207 encode the human C-peptide, which corresponds to amino acids 57-87 of NP_000198.
^{b}NM_008386 is a nucleotide sequence of murine insulin. Nucleotides 351-438 of NM_008386 encode the murine C-peptide, which corresponds to amino acids 57-85 of NP_032412.

The term “isolated”, as used in the context of a cell (including, for example, a fibroblast or a reprogrammed somatic cell of the presently disclosed subject matter), indicates that the cell exists apart from its native environment. An isolated cell can also exist in a purified form or can exist in a non-native environment.

As used herein, a cell exists in a “purified form” when it has been isolated away from all other cells that exist in its native environment, but also when the proportion of that cell in a mixture of cells is greater than would be found in its native environment. Stated another way, a cell is considered to be in “purified form” when the population of cells in question represents an enriched population of the cell of interest, even if other cells and cell types are also present in the enriched population. A cell can be considered in purified form when it comprises in some embodiments at least about 10% of a mixed population of cells, in some embodiments at least about 20% of a mixed population of cells, in some embodiments at least about 25% of a mixed population of cells, in some embodiments at least about 30% of a mixed population of cells, in some embodiments at least about 40% of a mixed population of cells, in some embodiments at least about 50% of a mixed population of cells, in some embodiments at least about 60% of a mixed population of cells, in some embodiments at least about 70% of a mixed population of cells, in some embodiments at least about 75% of a mixed population of cells, in some embodiments at least about 80% of a mixed population of cells, in some embodiments at least about 90% of a mixed population of cells, in some embodiments at least about 95% of a mixed population of cells, in some embodiments at least about 99% of a mixed population of cells, and in some embodiments about 100% of a mixed population of cells, with the proviso that the cell comprises a greater percentage of the total cell population in the “purified” population that it did in the population prior to the purification. In this respect, the terms “purified” and “enriched” can be considered synonymous.

As used herein, the phrase “sphere-induced Pluripotent Cells”, also referred to herein as “siPS cells” or “siPS”, refer to cells derived from embryoid body-like spheres produced from fibroblasts as set forth herein after replating and colony formation. The cells of the colonies, whether present in colonies or disaggregated therefrom, are referred to herein as siPS. In some embodiments, siPS form teratomas when transferred into nude mice. In some embodiments, siPS contribute to one or more lineages in chimeric mice when introduced into appropriate stage mouse embryos.

### II. Reprogrammed Somatic Cells and Methods for Producing the Same

The presently disclosed subject matter provides in some embodiments methods for producing a reprogrammed cell (e.g., a reprogrammed fibroblast).

As used herein, the term “reprogrammed”, and grammatical variants thereof, refers to a cell that has be manipulated in culture in order to acquire a degree of pluripotency that it would not have had if the manipulation in culture not taken place. Exemplary reprogrammed cells include, but are not limited to fibroblasts that as a result of the manipulations disclosed herein are induced to express markers associated with stem cells or with differentiated cells other than fibroblasts that the fibroblasts in culture do not and/or would not have expressed if maintained in monolayer culture.

Exemplary reprogrammed cells thus include the reprogrammed fibroblasts disclosed herein. In some embodiments, a reprogrammed fibroblast is a cell that has been isolated from an embryoid body-like sphere of the presently disclosed subject matter by sorting those cells that express certain markers associated with stem cells. In some embodiments, a reprogrammed fibroblast is a sphere-induced pluripotent cell (siPS) that has been produced by replating an embryoid body-like sphere of the presently disclosed subject matter under conditions sufficient for colony formation, wherein the colonies thus formed comprise reprogrammed fibroblasts. In some embodiments, a reprogrammed fibroblast is a cell line that has been generated from such a colony.

As used herein, the phrases “markers associated with stem cells”, “stem cell markers”, and “mRNA for stem cell markers” refer to genes the expression of which is generally associated with stem cells and other pluripotent and/or totipotent cells including, but not limited to embryonic stem (ES) cells and induced pluripotent cells (iPSC), but that that is not generally associated with reprogrammed cells in culture prior to the in vitro manipulation(s) that caused the cells to become reprogrammed. For example, the genes Oct4, Nanog, fibroblast growth factor-4 (FGF4), Sox2, Klf4, SSEA1, and Stat3 are all expressed by ES cells and other pluripotent cells, but are not expressed or expressed at a much lower level in fibroblasts. As such, they are referred to herein as “stem cell genes”, “genes associated with stem cells”, or “stem cell marker genes”. Upon reprogramming, fibroblasts upregulate one or more of these genes, and the upregulation of the one or more of these stem cell markers is in some embodiments indicative of reprogramming.

Thus, in some embodiments, the methods comprise (a) growing a plurality of cells (e.g., fibroblasts) in monolayer culture to confluency; and (b) disrupting the monolayer culture to place at least a fraction of the plurality of cells into suspension culture under conditions sufficient to form one or more embryoid body-like spheres, wherein the one or more embryoid body-like spheres comprise a reprogrammed cell induced to express at least one endogenous gene not expressed by the cell growing in the monolayer culture prior to the disrupting step.

As used herein, the phrase “conditions sufficient to form one or more embryoid body-like spheres” refers to any culture conditions wherein cells growing in monolayers that are disrupted initiate sphere formation while growing in suspension. Such conditions include various tissue culture media as well as different disruption techniques, examples of which are disclosed herein.

For example, in some embodiments the monolayers and/or the spheres that are generated therefrom are grown in a tissue culture medium. Tissue culture media that can be employed in the growth and maintenance of the cells and spheres of the presently disclosed subject matter include, but are not limited to any tissue culture medium that is generally used for growing and maintaining mammalian cells, particularly stem cells such as, but not limited to embryonic stem cells. Non-limiting examples of such media are DMEM, F12, RPMI-1640, and combinations thereof, which can be augmented with mammalian serum (e.g., 5-20% fetal bovine or fetal calf serum) and/or serum substitutes (e.g., OPTI-MEM® Reduced Serum Medium available from INVITROGEN™), glutamine and/or other essential amino acids, antibiotics and/or antimycotics, etc. as would be understood by one of ordinary skill in the art. Exemplary media that can be employed in the practice of the presently disclosed subject matter are disclosed in Nagy et al., 2003 and in U.S. Pat. Nos. 6,602,711; 7,153,684; and 7,220,584.

As used herein, the terms disrupted, “disruption”, and grammatical variants thereof refer to a manipulation of a monolayer of cells in culture that results in at least a subset of the monolayer detaching from the substrate upon which it is growing (and optionally, from other cells present in the monolayer) and growing in suspension. Mechanical methods of disruption including, but not limited to scraping a portion of the monolayer off a tissue culture plate, can be employed. Non-limiting examples of other disruption strategies include using light trypsinization and/or collagenase treatment to remove sheets of cells and scraping of monolayer cells followed by moderate pipetting with a pipetting device to dissociate the cells into smaller aggregates.

Thus, the term “disrupted” refers to a physical manipulation of the monolayer such that a plurality of cells becomes detached from the rest of the monolayer and from the growth surface and grows in suspension. The disruption can be anything that causes pluralities of cells as a unit to detach from the growth surface and grow in suspension. In some embodiments, the disrupting comprises scraping at least a fraction of the confluent monolayer off of a substrate upon which the confluent monolayer is being cultured.

Alternatively or in addition, a hanging drop method wherein lightly trypsinized cells in suspension are allowed to adhere to the underside of a tissue culture plate top can also be employed. Subsequently (in some embodiments one day later), the drops can be removed and placed in suspension culture. This procedure has been employed with ES cells to produced uniformly sized spheres or embryoid bodies, and can also be employed with the methods and compositions of the presently disclosed subject matter.

In some embodiments, a reprogrammed cell of the presently disclosed subject matter has the property of long term self-renewal. The phrase “long term self-renewal” refers to an ability to self-renew in culture over a period of in some embodiments at least one month, in some embodiments at least two months, in some embodiments at least three months, in some embodiments at least four months, in some embodiments at least five months, in some embodiments at least six months, and in some embodiments longer.

In some embodiments, a cell of the presently disclosed subject matter is a fibroblast. Fibroblasts can come from many sources from various species. In some embodiments, the fibroblast is a mammalian fibroblast, optionally a human fibroblast. Methods for isolating fibroblasts from various species are also known.

In some embodiments, the cell is selected from the group including adult human skin fibroblasts, adult peripheral blood mononuclear cells, adult human bone marrow-derived mononuclear cells, neonatal human skin fibroblasts, human umbilical vein endothelial cells, human umbilical artery smooth muscle cells, human postnatal skeletal muscle cells, human postnatal adipose cells, human postnatal peripheral blood mononuclear cells, or human cord blood mononuclear cells.

In some embodiments, a fibroblast is isolated from a source and grown in culture without any genetic manipulation (i.e., without the introduction of any exogenous coding and/or regulatory sequences using recombinant DNA technology). Thus, in such embodiments the cell (i.e., the fibroblast) is referred to as a non-recombinant cell.

Alternatively, a cell can be genetically manipulated by introducing into the cell one or more exogenous nucleic acid sequences. The exogenous nucleic acid sequences can include coding sequences. Alternatively or in addition, the exogenous nucleic acid sequence can include one or more regulatory sequences designed to regulate the expression of the exogenous coding sequences, endogenous coding sequences present in the cell, or both.

As such, in order to create one or more embryoid body-like spheres from cells (e.g., fibroblasts) growing in monolayer culture, the monolayers are disrupted to place at least a fraction of the fibroblasts into suspension culture. As the disrupted cells (e.g., fibroblasts) grow in suspension culture, they can form one or more embryoid body-like spheres. As used herein, the phrase “embryoid body-like sphere” refers to an aggregate of disrupted cells that appears morphologically similar to an embryoid body formed by embryonic stem (ES) cells under appropriate in vitro culturing conditions (see e.g., Nagy et al., 2003; U.S. Pat. No. 5,914,268). These embryoid body-like spheres are stable in culture; in some embodiments, they can be maintained in suspension culture for at least one month, and in some embodiments, they can be maintained in suspension culture for at least two months. In some embodiments, the one or more embryoid body-like spheres are maintained in a medium comprising Dulbecco's Modified Eagle Medium (DMEM) and 10% fetal bovine serum (FBS).

Upon formation of embryoid body-like spheres, some of the cells present therein are reprogrammed cells (in some embodiments, reprogrammed fibroblasts). The reprogrammed cells can be characterized by the expression of one or more stem cell markers that are not expressed (or are expressed to a much lower degree) by the cells (e.g., fibroblasts) in monolayer culture prior to formation of the embryoid body-like sphere. In some embodiments, the reprogrammed fibroblasts express at least one stem cell marker selected from the group including, but not limited to Oct4, Nanog, FGF4, Sox2, Klf4, Ssea1, and Stat3. Reagents that can be employed to assay for the expression of these stem cell markers and others include oligonucleotide primers comprising the sequences set forth in Table 1 herein above (e.g., for use in expression assays such as the RT-PCR assay). Like ES cells, the reprogrammed fibroblasts of the presently disclosed subject matter form teratomas in nude mice.

Since reprogrammed cells (e.g., fibroblasts) express certain stem cell markers that are not expressed by the cells absent reprogramming (or are expressed at a much lower level), the presently disclosed subject matter also provides methods for inducing expression of one or more stem cell markers in a cell (in some embodiments, a fibroblast). In some embodiments, the methods comprise (a) growing a plurality of cells in monolayer culture to confluency; and (b) disrupting the monolayer culture to place at least a fraction of the plurality of cells into suspension culture under conditions sufficient to form one or more spheres, wherein the one or more spheres comprise a cell with upregulated expression of one or more stem cell markers.

The presently disclosed subject matter also provides reprogrammed cells produced by the presently disclosed methods, reprogrammed cells non-recombinantly induced to express one or more endogenous stem cell markers, embryoid body-like spheres comprising a plurality of reprogrammed cells, and cell cultures comprising the presently disclosed embryoid body-like spheres in a medium sufficient to maintain the embryoid body-like spheres in suspension culture for at least one month. In some embodiments, the cells are fibroblasts.

Once formed, reprogrammed cells (e.g., fibroblasts) can be manipulated in vitro to differentiate into cell types of interest. Thus, the presently disclosed subject matter also provides methods for differentiating a reprogrammed cell into a cell type of interest. In some embodiments, the methods comprise (a) providing an embryoid body-like sphere comprising reprogrammed cells; and (b) culturing the embryoid body-like sphere in a culture medium comprising a differentiation-inducing amount of one or more factors that induce differentiation of the reprogrammed cells or derivatives thereof into the cell type of interest until the cell type of interest appears in the culture.

The reprogrammed cells of the presently disclosed subject matter can thus be differentiated into cell-types of various lineages, if desired. Examples of differentiated cells include any differentiated cells from ectodermal (e.g., neurons and fibroblasts), mesodermal (e.g., cardiomyocytes), or endodermal (e.g., pancreatic cells) lineages. By way of further example and not limitation, the differentiated cells can be in some embodiments pancreatic beta cells, in some embodiments neural stem cells, in some embodiments neurons (including, but not limited to dopaminergic neurons), in some embodiments oligodendrocytes, in some embodiments oligodendrocyte progenitor cells, in some embodiments hepatocytes, in some embodiments hepatic stem cells, in some embodiments astrocytes, in some embodiments myocytes, in some embodiments hematopoietic cells, and in some embodiments cardiomyocytes.

The differentiated cells derived from the reprogrammed cells of the presently disclosed subject matter can in some embodiments be terminally differentiated cells, or they can in some embodiments be capable of giving rise to cells of a specific lineage. For example, reprogrammed cells of the presently disclosed subject matter can be differentiated into a variety of multipotent cell types; e.g., neural stem cells, cardiac stem cells, and/or hepatic stem cells. These stem cells can then be further differentiated into new cell types, e.g., neural stem cells can be differentiated into neurons; cardiac stem cells can be differentiated into cardiomyocytes; and hepatic stem cells can be differentiated into hepatocytes.

There are numerous methods for differentiating the reprogrammed cells of the presently disclosed subject matter into more specialized cell types. Methods of differentiating reprogrammed cells can be similar to and based on those methods used to differentiate stem cells, particularly ES cells, mesenchymal stem cells (MSCs), multipotent adult progenitor cells (MAPCs), Marrow-isolated adult multilineage inducible cells (MIAMI cells), and hematopoietic stem cells (HSCs). In some embodiments, the differentiation occurs ex vivo; in some embodiments the differentiation occurs in vivo.

Any known method for generating neural stem cells from ES cells can be used to generate neural stem cells from the presently disclosed reprogrammed cells (see e.g., Reubinoff et al., 2001). For example, neural stem cells can be generated by culturing the reprogrammed cells of the presently disclosed subject matter in the presence of noggin and/or other bone morphogenetic protein antagonists (see e.g., Itsykson et al., 2005). In some embodiments, neural stem cells can be generated by culturing the reprogrammed cells of the presently disclosed subject matter in the presence of growth factors including, but not limited to FGF-2 (see Zhang et al., 2001). In some embodiments, the cells are cultured in serum-free medium containing FGF-2. In some embodiments, the reprogrammed cells of the presently disclosed subject matter are co-cultured with a mouse stromal cell line (e.g., the PA6 mouse stromal cell line) in the presence of serum-free medium comprising FGF-2 (see e.g., Kawasaki et al., 2000). In some embodiments, the reprogrammed cells of the presently disclosed subject matter are directly transferred to serum-free medium containing FGF-2 to directly induce differentiation.

Neural stems derived from the reprogrammed cells of the presently disclosed subject matter can be differentiated into neurons, oligodendrocytes, and/or astrocytes. Often, the conditions used to generate neural stem cells can also be used to generate neurons, oligodendrocytes, and/or astrocytes.

Dopaminergic neurons play a central role in Parkinson's Disease and other neurodegenerative diseases and are thus of particular interest. In order to promote differentiation into dopaminergic neurons, reprogrammed cells of the presently disclosed subject matter can be co-cultured with the PA6 mouse stromal cell line under serum-free conditions (see e.g., Kawasaki et al., 2000). Other methods have also been described in, for example, Pomp et al., 2005; U.S. Pat. No. 6,395,546; Lee et al., 2000.

Oligodendrocytes can also be generated from the reprogrammed cells of the presently disclosed subject matter. Differentiation of the reprogrammed cells of the presently disclosed subject matter into oligodendrocytes can be accomplished by methods that can be employed for differentiating ES cells or neural stem cells into oligodendrocytes. For example, oligodendrocytes can be generated by co-culturing reprogrammed cells of the presently disclosed subject matter and/or neural stem cells derived therefrom with stromal cells (see e.g., Hermann et al., 2004). In some embodiments, oligodendrocytes can be generated by culturing the reprogrammed cells of the presently disclosed subject matter and/or neural stem cells derived therefrom in the presence of a fusion protein in which the Interleukin (IL)-6 receptor or a biologically functional derivative thereof is linked to the IL-6 cytokine or a biologically functional derivative thereof. Oligodendrocytes can also be generated from the reprogrammed cells of the presently disclosed subject matter by other methods known in the art (see e.g. Kang et al., 2007).

Astrocytes can also be produced from the reprogrammed cells of the presently disclosed subject matter. Astrocytes can be generated by culturing reprogrammed cells of the presently disclosed subject matter and/or neural stem cells derived therefrom in the presence of neurogenic medium with bFGF and EGF (see e.g., Brustle et al., 1999).

Reprogrammed cells of the presently disclosed subject matter can be differentiated into pancreatic beta cells by methods known in the art (see e.g., Assady et al., 2001; Lumelsky et al., 2001; D'Amour et al., 2005; D'Amour et al., 2006). By way of example and not limitation, in some embodiments the methods can comprise culturing the reprogrammed cells of the presently disclosed subject matter in serum-free medium supplemented with Activin A, followed by culturing in the presence of serum-free medium supplemented with all-trans retinoic acid, followed by culturing in the presence of serum-free medium supplemented with bFGF and nicotinamide (see e.g., Jiang et al., 2007). In some embodiments, the method comprises culturing the reprogrammed cells of the presently disclosed subject matter in the presence of serum-free medium, activin A, and Wnt protein from about 0.5 to about 6 days, e.g., about 0.5, 1, 2, 3, 4, 5, 6, days; followed by culturing in the presence of from about 0.1% to about 2%, e.g., 0.2%, FBS and activin A from about 1 to about 4 days, e.g., about 1, 2, 3, or 4 days; followed by culturing in the presence of 2% FBS, FGF-10, and KAAD-cyclopamine (keto-N-aminoethylaminocaproyl dihydro cinnamoylcyclopamine) and retinoic acid from about 1 to about 5 days, e.g., 1, 2, 3, 4, or 5 days; followed by culturing with 1% B27, gamma secretase inhibitor and extendin-4 from about 1 to about 4 days, e.g., 1, 2, 3, or 4 days; and finally culturing in the presence of 1% B27, extendin-4, IGF-1, and HGF for from about 1 to about 4 days, e.g., 1, 2, 3, or 4 days.

Hepatic cells and/or hepatic stem cells can be differentiated from the reprogrammed cells of the presently disclosed subject matter. For example, culturing the reprogrammed cells of the presently disclosed subject matter in the presence of sodium butyrate can generate hepatocytes (see e.g., Rambhatla et al., 2003). In some embodiments, hepatocytes can be produced by culturing the reprogrammed cells of the presently disclosed subject matter in serum-free medium in the presence of Activin A, followed by culturing the cells in fibroblast growth factor-4 and bone morphogenetic protein-2 (see e.g., Cai et al., 2007). In some embodiments, the reprogrammed cells of the presently disclosed subject matter can be differentiated into hepatic cells and/or hepatic stem cells by culturing the reprogrammed cells of the presently disclosed subject matter in the presence of Activin A from about 2 to about 6 days, e.g., about 2, about 3, about 4, about 5, or about 6 days, and then culturing the reprogrammed cells of the presently disclosed subject matter in the presence of hepatocyte growth factor (HGF) for from about 5 days to about 10 days, e.g., about 5, about 6, about 7, about 8, about 9, or about 10 days.

The reprogrammed cells of the presently disclosed subject matter can also be differentiated into cardiac muscle cells. Inhibition of bone morphogenetic protein (BMP) signaling can result in the generation of cardiac muscle cells or cardiomyocytes (see e.g., Yuasa et al., 2005). Thus, in some embodiments, the reprogrammed cells of the presently disclosed subject matter are cultured in the presence of noggin for from about two to about six days, e.g., about 2, about 3, about 4, about 5, or about 6 days, prior to allowing formation of an embryoid body, and culturing the embryoid body for from about 1 week to about 4 weeks, e.g., about 1, about 2, about 3, or about 4 weeks.

In some embodiments, cardiomyocytes can be generated by culturing the reprogrammed cells of the presently disclosed subject matter in the presence of leukemia inhibitory factor (LIF), or by subjecting them to other methods known in the art to generate cardiomyocytes from ES cells (see e.g., Bader et al., 2000; Kehat et al., 2001; Mummery et al., 2003).

Examples of methods to generate other cell-types from reprogrammed cells of the presently disclosed subject matter include:

(1) culturing reprogrammed cells of the presently disclosed subject matter in the presence of retinoic acid, leukemia inhibitory factor (LIF), thyroid hormone (T3), and insulin in order to generate adipocytes (see e.g., Dani et al., 1997);

(2) culturing reprogrammed cells of the presently disclosed subject matter in the presence of BMP-2 or BMP-4 to generate chondrocytes (see e.g., Kramer et al., 2000);

(3) culturing the reprogrammed cells of the presently disclosed subject matter under conditions to generate smooth muscle (see e.g., Yamashita et al., 2000);

(4) culturing the reprogrammed cells of the presently disclosed subject matter in the presence of β1 integrin to generate keratinocytes (see e.g., Bagutti et al., 1996);

(5) culturing the reprogrammed cells of the presently disclosed subject matter in the presence of Interleukin-3 (IL-3) and macrophage colony stimulating factor to generate macrophages (see e.g., Lieschke & Dunn, 1995);

(6) culturing the reprogrammed cells of the presently disclosed subject matter in the presence of IL-3 and stem cell factor to generate mast cells (see e.g., Tsai et al., 2000);

(7) culturing the reprogrammed cells of the presently disclosed subject matter in the presence of dexamethasone and stromal cell layer, steel factor to generate melanocytes (see e.g., Yamane et al., 1999);

(8) co-culturing the reprogrammed cells of the presently disclosed subject matter with fetal mouse osteoblasts in the presence of dexamethasone, retinoic acid, ascorbic acid, and β-glycerophosphate to generate osteoblasts (see e.g., Buttery et al., 2001);

(9) culturing the reprogrammed cells of the presently disclosed subject matter in the presence of osteogenic factors to generate osteoblasts (see e.g., Sottile et al., 2003);

(10) overexpressing insulin-like growth factor-2 in the reprogrammed cells of the presently disclosed subject matter and culturing the cells in the presence of dimethyl sulfoxide to generate skeletal muscle cells (see e.g., Prelle et al., 2000);

(11) subjecting the reprogrammed cells of the presently disclosed subject matter to conditions for generating white blood cells; or

(12) culturing the reprogrammed cells of the presently disclosed subject matter in the presence of BMP4 and one or more: SCF, FLT3, IL-3, IL-6, and GCSF to generate hematopoietic progenitor cells (see e.g., Chadwick et al., (2003).

Thus, in some embodiments, a reprogrammed cell of the presently disclosed subject matter can be differentiated into a cell type of interest selected from the group including, but not limited to a neuronal cell, an endodermal cell, a cardiomyocyte, and derivatives thereof.

In some embodiments, the cell type of interest is a neuronal cell or a derivative thereof. In some embodiments, the neuronal cell or derivative thereof is selected from the group including, but not limited to an oligodendrocyte, an astrocyte, a glial cell, and a neuron. In some embodiments, the neuronal cell or derivative thereof expresses a marker selected from the group including, but not limited to GFAP, nestin, β III tubulin, Olig1, and Olig2. In some embodiments, the culture medium comprises about 10 ng/ml rhEGF, about 20 ng/ml FGF2, and about 20 ng/ml NGF, optionally wherein the culturing is for at least about 10 days. Neuronal cells and/or derivatives thereof can be identified using techniques known in the art including, but not limited to the use of antibodies that bind to GFAP, nestin, β III tubulin, Olig1, and Olig2, and/or other neuronal cell markers, or Reverse Transcription PCR using oligonucleotides are specific for GFAP, nestin, β III tubulin, Olig1, and Olig2 and/or other genes expressed in neuronal cells or their derivatives. Exemplary oligonucleotides are set forth in Table 1 herein above.

In some embodiments, the cell type of interest is an endodermal cell or derivative thereof. Culture conditions that can give rise to endodermal cells and/or derivatives thereof from reprogrammed fibroblasts include, but are not limited to culturing an embryoid body-like sphere in a first culture medium comprising Activin A; and thereafter culturing the embryoid body-like sphere in a second culture medium comprising N2 supplement-A, B27 supplement, and about 10 mM nicotinamide. In some embodiments, the culturing in the first culture medium is for about 48 hours. In some embodiments, the culturing in the second culture medium is for at least about 12 days. Culturing under one or more of these conditions can be sufficient to cause a differentiated derivative of a reprogrammed fibroblast to express a marker selected from the group including, but not limited to Nkx6-1, Pdx 1, and C-peptide. Endodermal cells and/or derivatives thereof can be identified using techniques known in the art including, but not limited to the use of antibodies that bind to Nkx6-1, Pdx 1, and C-peptide, and/or other endodermal cell markers, or Reverse Transcription PCR using oligonucleotides are specific for Nkx6-1, Pdx 1, C-peptide, and/or other genes expressed in endodermal cells or their derivatives. Exemplary oligonucleotides are set forth in Table 1 herein above.

In some embodiments, the cell type of interest is a cardiomyocyte or a derivative thereof. To produce a cardiomyocyte or a derivative thereof, the culturing is in some embodiments for at least about 15 days, optionally, in a culture medium comprising a combination of basic fibroblast growth factor, vascular endothelial growth factor, and transforming growth factor β1 in an amount sufficient to cause a subset of the embryoid body-like sphere cells to differentiate into cardiomyocytes. Culturing under these conditions can lead to the cardiomyocyte or the derivative thereof expressing a marker selected from the group including, but not limited to Nkx2-5/Csx and GATA4. Cardiomyocytes and/or derivatives thereof can be identified using techniques known in the art including, but not limited to the use of antibodies that bind to Nkx2-5/Csx and GATA4, and/or other cardiomyocyte markers, or Reverse Transcription PCR using oligonucleotides are specific for Nkx2-5/Csx, GATA4, and/or other genes expressed in cardiomyocytes and/or their derivatives. Exemplary oligonucleotides are set forth in Table 1 herein above.

### III. Applications

III.A. Methods for Obtaining Cells to be Reprogrammed

Exemplary methods for obtaining somatic cells (e.g., human somatic cells) are well established. See e.g., Schantz & Ng, 2004. In some embodiments, the methods include obtaining a cellular sample (e.g., by a biopsy such as, but not limited to a skin biopsy), blood draw, and/or alveolar and/or other pulmonary lavage. It is to be understood that initial plating densities of cells obtained and/or prepared from a tissue can be varied based on such variables as expected viability or adherence of cells from the particular tissue. Methods for obtaining various types of somatic cells include, but are not limited to, the following exemplary methods.

Skin tissue containing the dermis is harvested, for example, from the back of a knee or buttock. The skin tissue is then incubated for 30 minutes at 37° C. in 0.6% trypsin/Dulbecco's Modified Eagle's Medium (DMEM)/F-12 with 1% antibiotics/antimycotics, with the inner side of the skin facing downward.

After the skin tissue is turned over, tweezers are used to lightly scrub the inner side of the skin. The skin tissue is finely cut into 1 mm² sections and is then centrifuged at 1200 rpm for 10 minutes at room temperature. The supernatant is removed, and 25 ml of 0.1% trypsin/DMEM/F-12/1% antibiotics, antimycotics, is added to the tissue precipitate. The mixture is stirred at 200-300 rpm using a stirrer at 37° C. for 40 minutes. After confirming that the tissue precipitate is fully digested, 3 ml fetal bovine serum (FBS) is added, and filtered sequentially with gauze, a 100 μm nylon filter, and a 40 μm nylon filter. After centrifuging the resulting filtrate at 1200 rpm for 10 minutes at room temperature to remove the supernatant, DMEM/F-12/1% antibiotics, antimycotics is added to wash the precipitate, and then centrifuged at 1200 rpm at room temperature for 10 minutes. The cell fraction thus obtained is then cultured as described herein.

Dermal cells can be enriched by isolating dermal papilla from scalp tissue. By way of example and not limitation, human scalp tissue (0.5-2 cm² or less) is rinsed, trimmed to remove excess adipose tissues, and cut into small pieces. These tissue pieces are enzymatically digested in 12.5 mg/ml dispase (INVITROGEN™, Carlsbad, Calif., United States of America) in DMEM for 24 hours at 4° C. After the enzymatic treatment, the epidermis is peeled from the dermis and hair follicles are removed from the dermis. Hair follicles are washed with phosphate-buffered saline (PBS) and the epidermis and dermis are removed. A microscope can be used for this procedure. Single dermal-papilla derived cells are generated by culturing the explanted papilla on a plastic tissue culture dish in the medium containing DMEM and 10% fetal calf serum (FCS) for 1 week. When single dermal papilla cells are generated, these cells are removed and cultured in FBM supplemented with FGM-2 SINGLEQUOTS® (Lonza Inc., Allendale, N.J., United States of America) or cultured in the presence of 20 ng/ml EGF, 40 ng/ml FGF-2, and B27 without serum.

Epidermal cells can be also enriched, for example, from human scalp tissue (0.5-2 cm² or less). Human scalp tissue is rinsed, trimmed to remove excess adipose tissues, and cut into small pieces. These tissue pieces are enzymatically digested in 12.5 mg/ml dispase (INVITROGEN™) in Dulbecco's modified Eagle's medium (DMEM) for 24 hours at 4° C. After the enzymatic treatment, the epidermis is peeled off from the dermis; and hair follicles are pulled out from the dermis. The bulb and intact outer root sheath (ORS) are dissected under a microscope. After the wash, the follicles are transferred into a plastic dish. Then the bulge region is dissected from the upper follicle using a fine needle. After the wash, the bulge is transferred into a new dish and cultured in medium containing DMEM/F12 and 10% FBS. After the cells are identified, culture medium is changed to the EPILIFE™ Extended-Lifespan Serum-Free Medium (Sigma-Aldrich Corp., St. Louis, Mo., United States of America).

III.B. Methods of Treatment

The presently disclosed subject matter provides in some embodiments methods for treating a disease, disorder, and/or injury to a tissue in a subject. In some embodiments, the methods comprise administering to the subject a composition comprising a plurality of reprogrammed cells (e.g., fibroblasts) in a pharmaceutically acceptable carrier in an amount and via a route sufficient to allow at least a fraction of the reprogrammed cells to engraft the target tissue and differentiate therein, whereby the disease, disorder, and/or injury is treated. The disease, disorder, and/or injury can be any disease, disorder, and/or injury in which cell replacement therapy might be expected to be beneficial. As such, in some embodiments the disease, disorder, and/or injury is selected from the group including, but not limited to an ischemic injury, a myocardial infarction, and stroke.

The terms “target tissue” and “target organ” as used herein refer to an intended site for accumulation of a reprogrammed cell of the presently disclosed subject matter and/or a differentiated derivative thereof (e.g., an in vitro differentiated derivative thereof) following administration to a subject. For example, in some embodiments the methods of the presently disclosed subject matter involve a target tissue or a target organ that has been damaged, for example by ischemia or other injury.

The term “control tissue” as used herein refers to a site suspected to substantially lack accumulation of an administered cell. For example, in accordance with the methods of the presently disclosed subject matter, a tissue or organ that has not been injured or damaged is a representative control tissue, as is a tissue or organ other than the intended target tissue.

The terms “targeting” and “homing”, as used herein to describe the in vivo activity of a cell (for example, a reprogrammed cell of the presently disclosed subject matter and/or an in vitro differentiated derivative thereof) following administration to a subject, and refer to the preferential movement and/or accumulation of the cell in a target tissue as compared to a control tissue.

The terms “selective targeting” and “selective homing” as used herein refer to a preferential localization of a cell (for example, a reprogrammed cell of the presently disclosed subject matter and/or an in vitro differentiated derivative thereof) that results in an accumulation of the administered reprogrammed cell of the presently disclosed subject matter and/or an in vitro differentiated derivative thereof in a target tissue that is in some embodiments about 2-fold greater than accumulation of the administered reprogrammed cell of the presently disclosed subject matter and/or an in vitro differentiated derivative thereof in a control tissue, in some embodiments accumulation of the administered reprogrammed cell of the presently disclosed subject matter and/or an in vitro differentiated derivative thereof that is about 5-fold or greater, and in some embodiments an accumulation of the administered reprogrammed cell of the presently disclosed subject matter and/or an in vitro differentiated derivative thereof that is about 10-fold or greater than in an control tissue. The terms “selective targeting” and “selective homing” also refer to accumulation of a reprogrammed cell of the presently disclosed subject matter and/or an in vitro differentiated derivative thereof in a target tissue concomitant with an absence of accumulation in a control tissue, in some embodiments the absence of accumulation in all control tissues. Techniques that can be employed for targeting reprogrammed cells of the presently disclosed subject matter are disclosed in PCT International Patent Application Publication Nos. WO 2007/067280 and WO 2009/059032, the disclosure of each of which is incorporated by reference herein in its entirety.

The term “absence of targeting” is used herein to describe substantially no binding or accumulation of a reprogrammed cell of the presently disclosed subject matter and/or an in vitro differentiated derivative thereof in one or more control tissues under conditions wherein accumulation would be detectable if present. The phrase also is intended to include minimal, background accumulation of a reprogrammed cell of the presently disclosed subject matter and/or an in vitro differentiated derivative thereof in one or more control tissues under such conditions.

In some embodiments, the administering is of a reprogrammed cell, or a differentiated derivative thereof, which is from a donor. In some embodiments, the donor is the same individual as the recipient, but in some embodiments the donor is a different individual. In the case of different donors and recipients, the donor can be immunocompatible with the recipient. In some embodiments, the donor is identified as immunocompatible if the HLA genotype matches the HLA genotype of the recipient. In some embodiments, the immunocompatible donor is identified by genotyping a blood sample from the immunocompatible donor.

Depending on the nature of the injury to be treated, the methods can further comprise differentiating the reprogrammed cells (e.g., fibroblasts) to produce a pre-determined cell type prior to administering the composition to the subject. For example, the pre-determined cell type can be selected from the group including, but not limited to a neural cell, an endoderm cell, a cardiomyocyte, and derivatives thereof, although the presently disclosed subject matter is not limited to just these cell types of interest.

III.B.1. Formulations

The compositions of the presently disclosed subject matter comprise in some embodiments a composition that includes an active agent (e.g., a reprogrammed cell and/or a derivative thereof, as well as pluralities thereof) and a carrier, particularly a pharmaceutically acceptable carrier, such as but not limited to a carrier pharmaceutically acceptable for use in humans. Any suitable pharmaceutical formulation can be used to prepare the compositions for administration to a subject.

For example, suitable formulations can include aqueous and non-aqueous sterile injection solutions that can contain anti-oxidants, buffers, bacteriostatics, bactericidal antibiotics, and solutes that render the formulation isotonic with the bodily fluids of the intended recipient.

It should be understood that in addition to the ingredients particularly mentioned above the formulations of the presently disclosed subject matter can include other agents conventional in the art with regard to the type of formulation in question. For example, sterile pyrogen-free aqueous and non-aqueous solutions can be used.

The therapeutic regimens and compositions of the presently disclosed subject matter can be used with additional adjuvants and/or biological response modifiers (BRMs) including, but not limited to, cytokines and other immunomodulating compounds. Exemplary adjuvants and/or biological response modifiers include, but are not limited to monoclonal antibodies, interferons (IFNs, including but not limited to IFN-α and IFN-γ), interleukins (ILs, including but not limited to IL2, IL4, IL6, and IL10), cytokines (including, but not limited to tumor necrosis factors), and colony-stimulating factors (CSFs, including by not limited to GM-CSF and GCSF).

III.B.2. Administration

Suitable methods for administration of the compositions of the presently disclosed subject matter include, but are not limited to intravenous administration and delivery directly to the target tissue or organ. In some embodiments, the method of administration encompasses features for regionalized delivery or accumulation of the compositions of the presently disclosed subject matter at the site in need of treatment. In some embodiments, the compositions of the presently disclosed subject matter are delivered directly into the tissue or organ to be treated. In some embodiments, selective delivery of the cells present in the compositions of the presently disclosed subject matter is accomplished by intravenous injection of the presently disclosed compositions, where the cells present therein can home to the target tissue and/or organ and engraft therein.

III.B.3. Dose

An effective dose of a composition of the presently disclosed subject matter is administered to a subject in need thereof. A “treatment effective amount” or a “therapeutic amount” is an amount of a therapeutic composition sufficient to produce a measurable response (e.g., a biologically or clinically relevant response in a subject being treated). Actual dosage levels of an active agent or agents (e.g., a reprogrammed cell and/or a differentiated derivative thereof) in the compositions of the presently disclosed subject matter can be varied so as to administer an amount of the active agent(s) that is effective to achieve the desired therapeutic response for a particular subject. The selected dosage level will depend upon the activity of the therapeutic composition, the route of administration, combination with other drugs or treatments, the severity of the condition being treated, and the condition and prior medical history of the subject being treated. However, it is within the skill of the art to start doses of the compositions of the presently disclosed subject matter at levels lower than required to achieve the desired therapeutic effect and to gradually increase the dosage until the desired effect is achieved. The potency of a composition can vary, and therefore a “treatment effective amount” can vary. However, one skilled in the art can readily assess the potency and efficacy of a therapeutic composition of the presently disclosed subject matter and adjust the therapeutic regimen accordingly.

After review of the disclosure of the presently disclosed subject matter presented herein, one of ordinary skill in the art can tailor the dosages to an individual subject, taking into account the particular formulation, method of administration to be used with the composition, and particular injury treated. Further calculations of dose can consider subject height and weight, severity and stage of symptoms, and the presence of additional deleterious physical conditions. Such adjustments or variations, as well as evaluation of when and how to make such adjustments or variations, are well known to those of ordinary skill in the art.

### IV. Production of Chimeric and Transgenic Animals and Animals Produced Thereby

In some embodiments, the presently disclosed subject matter provides methods for producing chimeric non-human vertebrate animals including, but not limited to, mice. General methods for producing chimeric non-human vertebrate animals by transfer of pluripotent cells into host embryos are known to one of ordinary skill in the art (see e.g., Stewart, 1993; Saburi et al., 1997; Papaioannou & Johnson, 2000; Nagy et al., 2003), and can be implemented to employ the sphere-induced Pluripotent Cells (siPS) of the presently disclosed subject matter.

For example, in some embodiments the presently disclosed subject matter provides methods for producing chimeric non-human vertebrate animals comprising transferring one or more siPS into a host embryo, implanting the host embryo into an embryo recipient (such as, but not limited to a pseudopregnant female animal), and allowing the host embryo to be born, wherein a chimeric non-human vertebrate animal (e.g., a mouse) is produced. In some embodiments, the chimeric non-human vertebrate animal comprises one or more somatic and/or germ cells that are derived from (i.e., are progeny cells of) one or more of the siPS that were transferred into the host embryo. In some embodiments, the one or more siPS transferred into the host embryo are produced as set forth herein. The transferring step can comprise transferring at least one, two, three, four, five, six, seven, eight, nine, ten, eleven, twelve, thirteen, fourteen, fifteen, sixteen, seventeen, eighteen, nineteen, twenty, or even more siPS into the host embryo. In some embodiments, the host embryo is a morula stage embryo or a blastocyst stage embryo.

Subsequent to transfer of the siPS into the host embryo, the host embryos can then be implanted into an embryo recipient (e.g., a pseudopregnant female animal such as, but not limited to a pseudopregnant female mouse), wherein the embryo recipient is either pregnant or pseudopregnant at a stage of (pseudo)pregnancy appropriate for receiving the host embryos and bringing them to term. Methods for inducing (pseudo)pregnancy are known to those of skill (see Nagy et al., 2003). For example, when a host embryo is a blastocyst stage embryo, the embryo recipient can be mated with sterile males to produce a pseudopregnant female, which in the case of pseudopregnant female mice, can serve as a blastocyst stage embryo recipient at day 2.5 p.c. (day 0.5 p.c. being the morning after the mating has occurred).

In some embodiments, the implanted host embryos are allowed to develop to term and be born. In some embodiments, the animals that are born are tested for the presence of siPS-derived cells (e.g., cells that are progeny of the transferred siPS) in their somatic tissues and/or germline. In some embodiments, siPS-derived cells are identified in the germline of the chimeric animals, and in some embodiments, the chimeric mice are germline chimeric animals that can pass the SIPS-derived genomes or a fraction thereof to subsequent generations.

In some embodiments, the siPS are derived from fibroblasts that comprise at least one transgene. The term “transgene” is used herein to describe genetic material that has been or is about to be artificially inserted into the genome of a fibroblast of a warm-blooded vertebrate animal. In some embodiments, the transgene is operably linked to a promoter that is active in at least one cell type and/or developmental stage of the species from which the fibroblasts are derived to an extent sufficient to modify a phenotype of a chimeric animal produced by generating siPS from the fibroblasts and transferring the siPS to a host embryo as compared to a non-chimeric animal of the same genetic background as that of the host embryo.

The presently disclosed subject matter also provides chimeric animals (including, but not limited to chimeric mice) produced by the presently disclosed methods. As used herein, the phrase “chimeric animal” refers to an animal that results from the integration of one or more siPS and/or progeny cells thereof (referred to herein as “sphere-induced Pluripotent Cells (siPS)-derived cells”) into at least one somatic tissue, gonadal tissue, or both, wherein the one or more siPS were artificially introduced into the animal under conditions sufficient to result in the siPS and/or their mitotic and/or meiotic progeny taking part in the normal development of at least one tissue or cell type of the animal. As used herein, the phrase “chimeric animal” refers to any such animal at any stage of development. In some embodiments, the chimeric animal (e.g., the chimeric mouse) is a pre-term embryo. The chimeric animal can also be in some embodiments a juvenile animal and in some embodiments an adult animal.

In some embodiments, one or more siPS-derived cells are present within the germline of the chimeric animal, thereby producing a germline chimeric animal. As used herein, the phrase “sphere-induced Pluripotent Cells (siPS)-derived cells” in the context of cells present within an animal refers to cells that are daughter cells of siPS resulting from by the process of meiotic and/or mitotic division of siPS or are daughter cells resulting from the process of meiotic and/or mitotic division of daughter cells of siPS. Stated another way, in some embodiments siPS-derived cells are the developmental progeny of siPS and/or the developmental progeny of cells that themselves are developmental progeny of siPS.

### V. Other Applications

The presently disclosed subject matter also provides methods for analyzing differentiation of different cell lineages. As such, the reprogramming strategies disclosed herein, and the cells produced therewith, can be employed to study the differentiation of cells representative of all three embryonic layers. For example, the results disclosed herein with respect to erythrocytes and the Real Time PCR results demonstrating expression of early and late stage markers of differentiation demonstrated that reprogrammed cells progressed along pathways of differentiation under the disclosed conditions. Molecular events including sequential gene expression patterns as well as epigenetic changes in each of the cell types can be investigated using the compositions and methods of the presently disclosed subject matter.

The presently disclosed subject matter also provides methods for analyzing the transition of differentiated somatic cells to cancer stem cells during tumor formation and/or progression. Additionally, the present disclosure includes a large amount of data that demonstrates that mutations of the members of the RB1 family can lead to the generation of cells with properties of cancer stem cells. Mutations in RB family members are known to be important events in cancer, as most if not all cancers appear to inactivate one or more RB1 family members as a step toward transformation.

Thus, the compositions and methods of the presently disclosed subject matter can be employed as a model for RB1 family-dependent transition of cells (e.g., ES cells, iPSC, or other cells) to cancer stem cells. What gene expression changes regulate this transition and which epigenetic changes might be responsible for such changes in gene expression can be investigated using the presently disclosed subject matter. One such change in gene expression which can be examined for a role in the generation of cancer stem cells (dependent upon whether wild type or RB1-mutant cells are used) are the epithelial-mesenchymal transcription (EMT) factors including, but not limited to Zeb1.

Moreover, the presently disclosed subject matter can be employed in investigations of other events that might be responsible for transition of cells to cancer stem cells.

And finally, emerging evidence suggests that cancers can be initiated by an outgrowth of fully differentiated somatic cells into sphere-like structures with concomitant loss of cell-cell contact inhibition. Cells within these growing spheres undergo dedifferentiation to form cells with properties of cancer stem cells. As such, the methods and compositions of the presently disclosed subject matter could be employed as a model in culture and also in vivo in tumor formation models to define the steps in cancer formation that are initiated by outgrowth of differentiated somatic cells lacking cell-cell contact inhibition. In some embodiments, this could involve investigation of gene expression changes as well as epigenetic changes responsible for such alterations in gene expression.

### EXAMPLES

The presently disclosed subject matter will be now be described more fully hereinafter with reference to the accompanying EXAMPLES, in which representative embodiments of the presently disclosed subject matter are shown. The presently disclosed subject matter can, however, be embodied in different forms and should not be construed as limited to the embodiments set forth herein. Rather, these embodiments are provided so that this disclosure will be thorough and complete, and will fully convey the scope of the presently disclosed subject matter to those skilled in the art.

### Method and Materials for the EXAMPLES

Cells and cell culture: Wild type mouse embryo fibroblasts (MEFs) were isolated from embryonic day 13.5 (E13.5) mouse embryos, and Rb family mutant MEFs were kind gifts from Tyler Jacks (Massachusetts Institute of Technology, Cambridge, Mass., United States of America), Julien Sage (Stanford University, Palo Alto, Calif., United States of America), and Gustavo Leone (The Ohio State University, Columbus, Ohio, United States of America). Fibroblasts in which all three RB1 family members have been mutated (referred to herein as “triple knockouts” and “TKOs”) derived from four separate embryos were used in the experiments described herein with similar results. Cells were analyzed beginning at passage 4, but similar results were also seen at passage 11. The cells were cultured in DMEM with 10% heat-inactivated fetal bovine serum. One (1) unit/mL of leukemia inhibitory factor (LIF; CHEMICON® International, Inc., Temecula, Calif., United States of America) was added to embryonic stem cell cultures.

Immunohistochemistry. Exemplary primary and secondary antibodies employed herein are described in Tables 3 and 4. Primary antibodies were incubated at 4° C. overnight, and after three washes with phosphate-buffered saline (PBS), slides were incubated at 1:200 dilution with secondary antibodies conjugated with either Cy3 or ALEXA FLUOR® 488 (MOLECULAR PROBES®, a division of INVITROGEN™ Corp., Carlsbad, Calif., United States of America) at room temperature for 60 minutes. After three washes with PBS, slides were mounted with coverslips using either the anti-fade medium PERMOUNT™ (Fisher Scientific, Fair Lawn, N.J., United States of America) or VECTASHIELD® Mounting Medium with DAPI (Vector Laboratories, Inc., Burlingame, Calif., United States of America), and images were captured with an Olympus confocal microscope.

TABLE 3
Listing of Primary Antibodies Employed

| | IgG | Cross- | | |
|---|---|---|---|---|
| Specificity | Type¹ | reactivity² | Supplier | Dilution |
| AFP | goat (P) | m, r, h | Santa Cruz | 1:100 |
| Anti-E-cadherin | mouse (M) | m, r, | Douglas Darling | 1:50 |
| (Cdh1) | | h, d | (BD Biosciences | |
| | | | Pharmingen) | |
| BCRP/Abcg2 | rat (M) | m, r, h | Abcam | 1:20 |
| BRDU (G3G4) | mouse (P) | m, r, h | Douglas Darling | 1:50 |
| Calbindin-D-28K | rabbit (P) | h, m, r | Thermo Scientific | 1:500 |
| CD133 | rat (M) | m, r, h | CHEMICON ® | 1:50 |
| CD31 (PECAM) | mouse (M) | m, h | Tongalp Tezel | 1:50 |
| c-peptide | pig (P) | m, r, h | Millipore | 1:200 |
| GATA4 | mouse (M) | m, r, h | Santa Cruz | 1:100 |
| GFAP | mouse (M) | m, r, h | CHEMICON ® | 1:50 |
| hemoglobin (HB) | goat (P) | m, r, h | Tongalp Tezel | 1:50 |
| Insulin | pig (P) | m, r, h | Abcam | 1:200 |
| Islet1 | mouse (M) | m, r, h | Douglas Darling | 1:0 |
| MBP | mouse (M) | m, r, h | Abcam | 1:100 |
| mouse Nanog | rat (M) | m, r, h | EBIOSCIENCE ™ | 1:200 |
| Nanog | rat (M) | m, r, h | EBIOSCIENCE ™ | 1:20 |
| PKC alpha | mouse (M) | h, m, r, | Assay Designs | 1:500 |
| | | others | | |
| POU5F1 (Oct4) | rabbit (P) | m, r, h | Sigma | 1:20 |
| recoverin | rabbit (P) | h, m, r, | CHEMICON ® | 1:500 |
| | | c, f | | |
| Rhodopsin (Opsin) | mouse (M) | h, m, r | Thermo Scientific | 1:500 |
| sarcomeric actinin | mouse (M) | m, r, h | Abcam | 1:100 |
| SSEA1 | mouse (M) | m, r, h | CHEMICON ® | 1:100 |
| Synapsin-1 | rabbit (P) | h, m, r | INVITROGEN ™ | 1:500 |
| | | | (Myzel) | |
| TH alpha | mouse (M) | m, r, h | Douglas Darling | 1:0 |
| troponin I | mouse (M) | m, r, h | CHEMICON ® | 1:200 |
| vimentin | goat (P) | m, r, h | Santa Cruz | 1:50 |
| β-III tubulin | mouse (M) | m, r, h | CHEMICON ® | 1:50 |

¹(M)—monoclonal.
(P)—polyclonal.
²m—mouse;
r—rat;
h—human;
c—chick;
f—frog;
d—dog.
Abcam: Abcam Inc., Cambridge, Massachusetts, United States of America;
Assay Designs: Assay Designs, Inc., Ann Arbor, Michigan, United States of America;
CHEMICON ®: Chemicon Inc., a division of Millipore Corp., Billerica, Massachusetts, United States of America;
Doug Darling: Dental School University of Louisville, Louisville, Kentucky, United States of America;
EBIOSCIENCE ™: eBioscience, Inc., San Diego, California, United States of America;
INVITROGEN ™: INVITROGEN ™ Corp., Carlsbad, California, United States of America;
Millipore: Millipore Corp., Billerica, Massachusetts, United States of America;
Santa Cruz: Santa Cruz Biotechnology Inc., Santa Cruz, California, United States of America;
Sigma: Sigma-Aldrich Corp., St. Louis, Missouri, United States of America;
Thermo Scientific: Thermo Fischer Scientific Inc., Waltham, Massachusetts, United States of America;
Tongalp Tezel: Department of Ophthalmology and Visual Sciences, University of Louisville, Louisville, Kentucky, United States of America.

TABLE 4
Listing of Secondary Antibodies Employed

| Description | Manufacturer | Dilution |
|---|---|---|
| Cy3-conjugated Rabbit anti-rat IgG | CHEMICOM ® | 1:200 |
| ALEXA FLUOR ® 488-conjugated Goat | MOLECULAR | 1:200 |
| anti-mouse IgG | PROBES ® | |
| ALEXA FLUOR ® 488-conjugated Goat | MOLECULAR | 1:200 |
| anti-rabbit IgG | PROBES ® | |
| ALEXA FLUOR ® 488-conjugated | MOLECULAR | 1:200 |
| Donkey anti-goat IgG | PROBES ® | |
| Cy3-conjugated Sheep anti-rabbit IgG | Sigma | 1:200 |

Tumor formation in nude mice. Either spheres (after two weeks in suspension culture) or trypsinized monolayers of cells derived from spheres were injected subcutaneously into the right hind limb of Balb/cAnNCr-nu/nu nude mice (available from the National Cancer Institute at Fredrick, Frederick, Md., United States of America). Tumors were fixed in 10% buffered formalin, embedded in paraffin, sectioned at 5 μm, and stained with hematoxylin and eosin (H&E) and/or used for immunostaining.

Identification and isolation of Side Population (SP) and Main Population (MP) cells. Cells were trypsinized from tissue culture plates, suspended in pre-warmed DMEM containing 2% FBS and 10 mM 4-(2-hydroxyethyl)-1-piperazineethanesulfonic acid (HEPES), and stained with 5 g/ml of Hoechst 33342 dye (MOLECULAR PROBES®) for 90 minutes at 37° C. Cells were then washed and resuspended in Hank's Buffered Salt Solution (HBSS) containing 2% FBS and 10 mM HEPES. Before cell sorting, 2 g/ml propidium iodide (Sigma-Aldrich, Inc., St. Louis, Mo., United States of America) was added to exclude nonviable cells. SP cells were identified and isolated using a MOFLO™ cell sorter (Dako North America, Inc., Carpinteria, Calif., United States of America) after excitation of the Hoechst dye with a 350 nm UV laser (100 mW power was used). Fluorescence light emitted by cells was directed toward a 510 nm DCLP dichroic mirror and collected simultaneously by two independent detectors following a 450/65 nm and a 670/30 nm band pass filters, respectively. Cells were analyzed on a linearly amplified fluorescence scale.

For immunostaining, Hoechst 33342-treated cells were collected by centrifugation, washed twice with PBS, and incubated either with a rat anti-Abcg2 (1:20) or a mouse anti-CD133 (1:50) primary antibody for 1 hour at room temperature. No blocking serum was used. Cy3-conjugated anti-rat IgG (1:200; CHEMICON® International, Inc.) and ALEXA FLUOR® 488-conjugated anti-mouse IgG (1:200; MOLECULAR PROBES®) were the secondary antibodies for anti-Abcg2 and anti-CD133, respectively. Images were captured with an Olympus confocal microscope.

RNA extraction and Real Time PCR. RNA was extracted from spheres and/or cells using TRIZOL® reagent (INVITROGEN™ Corp.), and cDNA was synthesized using the INVITROGEN™ RT kit (INVITROGEN™ Corp.), and SYBR® Green Real Time PCR was performed using a Stratagene Mx3000P Real Time PCR system (Stratagene, La Jolla, Calif., United States of America). PCR primers are described in Table 1 herein above. A mouse stem cell Real Time PCR Array was also analyzed (Catalogue No. APMM-405, SABIOSCIENCES™ Corporation, Frederick, Md., United States of America). Three independent samples, each in triplicate, were analyzed for each Real Time PCR condition.

Lentivirus shRNA Methods. The shRNA oligomers used for Zeb1 and Zeb2 silencing were described previously (Nishimura et al., 2006). The shRNAs were first cloned into a CMV-GFP lentiviral vector where its expression was driven by the mouse U6 promoter.

Briefly, each shRNA construct was generated by synthesizing an 83-mer oligonucleotide containing: (i) a 19-nucleotide sense strand and a 19-nucleotide antisense strand separated by a nine-nucleotide loop (5′-TTCAAGAGA-3′); (ii) a stretch of five adenines as a template for the PolIII promoter termination signal; (iii) 21 nucleotides complimentary to the 3′ end of the PolIII U6 promoter; and (iv) a 5′ end containing a unique XbaI restriction site. The long oligonucleotide was used together with a SP6 oligonucleotide (5′-ATTTAGGTGACACTATAGAAT-3; SEQ ID NO: 71) to PCR-amplify a fragment containing the entire U6 promoter plus shRNA sequences. The resulting product was digested with XbaI and SpeI, ligated into the NheI site of the lentivirus vector, and the insert was sequenced to ensure that no errors had occurred during the PCR or cloning steps. The sequences of the 19-nucleotide sense strands were 5′-AAGACAACGTGAAAGACAA-3′ (SEQ ID NO: 72) for Zeb1 and 5′-GGAAAAACGTGGTGAACTA-3′ (SEQ ID NO: 73) for Zeb2. A negative control shRNA was also tested that had a sense strand of 5′-AACAAGATGAAGAGCACCA-3′ (SEQ ID NO: 74).

The detailed procedure is described in Tiscornia et al., 2006. Briefly, 293T cells were transfected with the lentiviral vector and packaging plasmids, and the supernatants containing recombinant pseudolentiviral particles were collected from culture dishes on the second and third days after transfection. MEFs were transduced with these lentiviral particles expressing shRNAs targeting Zeb1 or Zeb2 (or the negative control shRNA). A transduction efficiency of near 100% was achieved based on GFP-positive cells.

### Example 1

### RB1 Family Mutation Allows Outgrowth of Cells into Spheres Leading to Survival in Suspension and Stable Changes in Cell Morphology

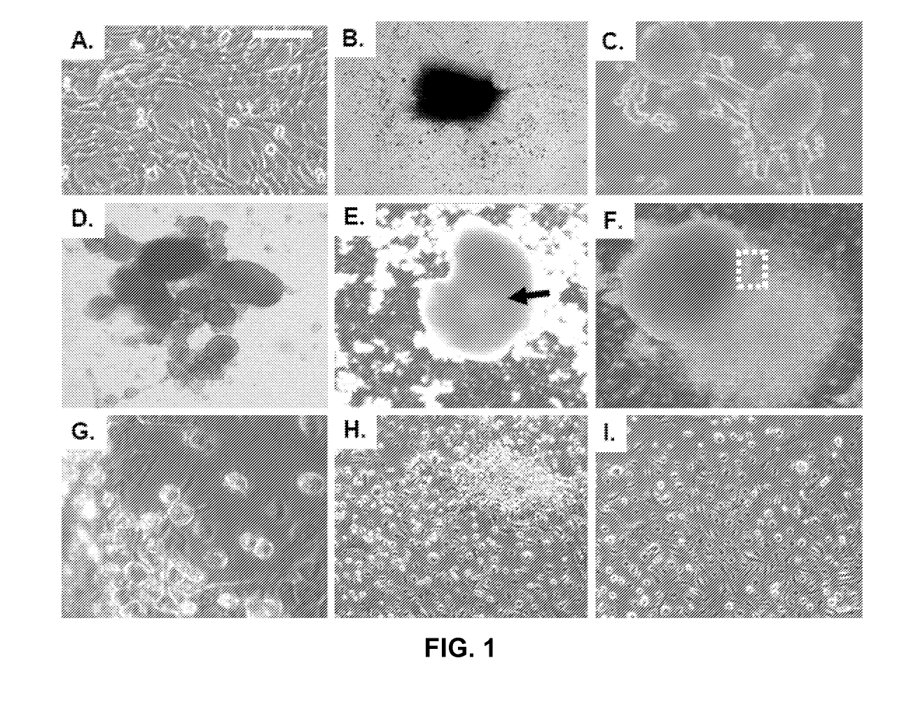

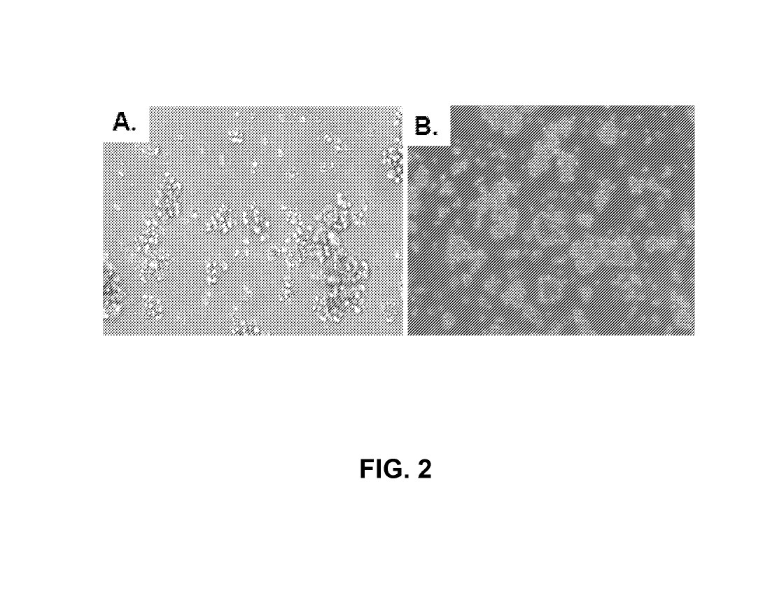

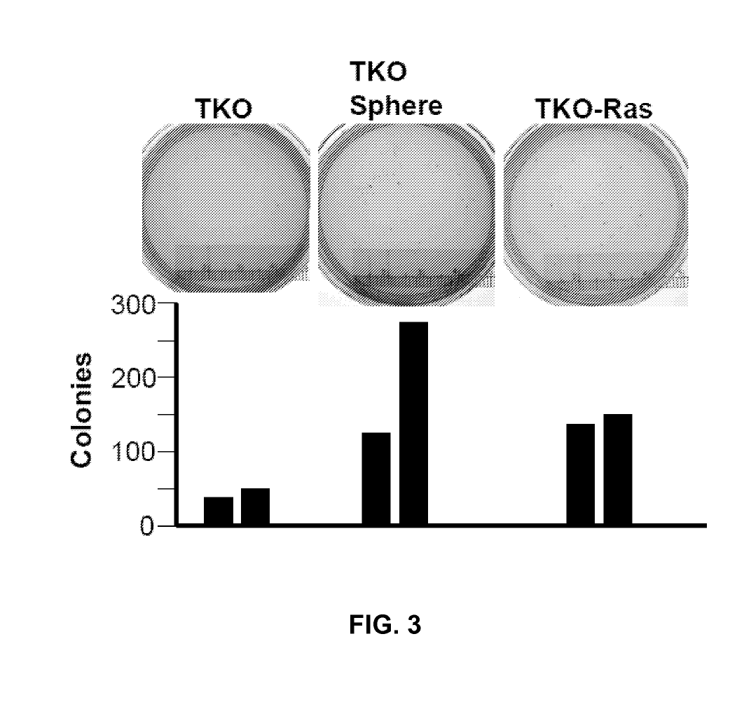

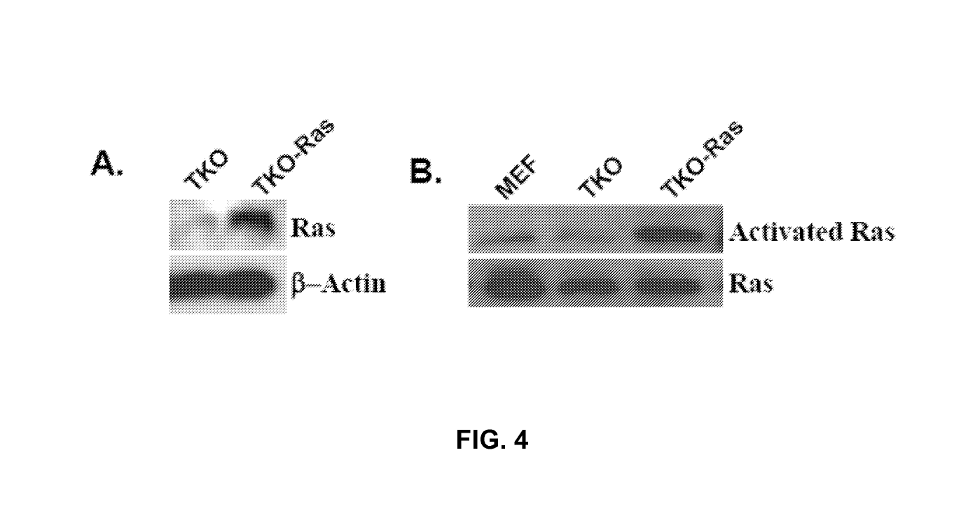

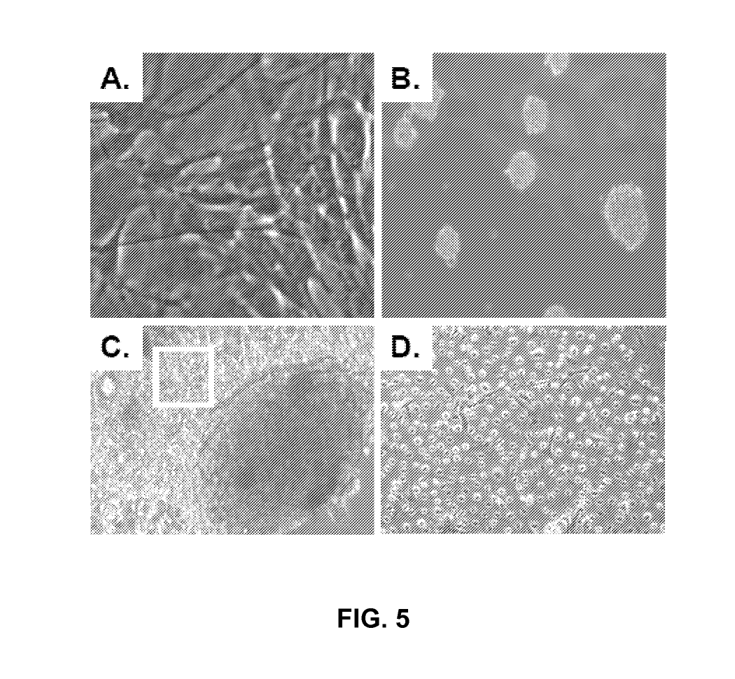

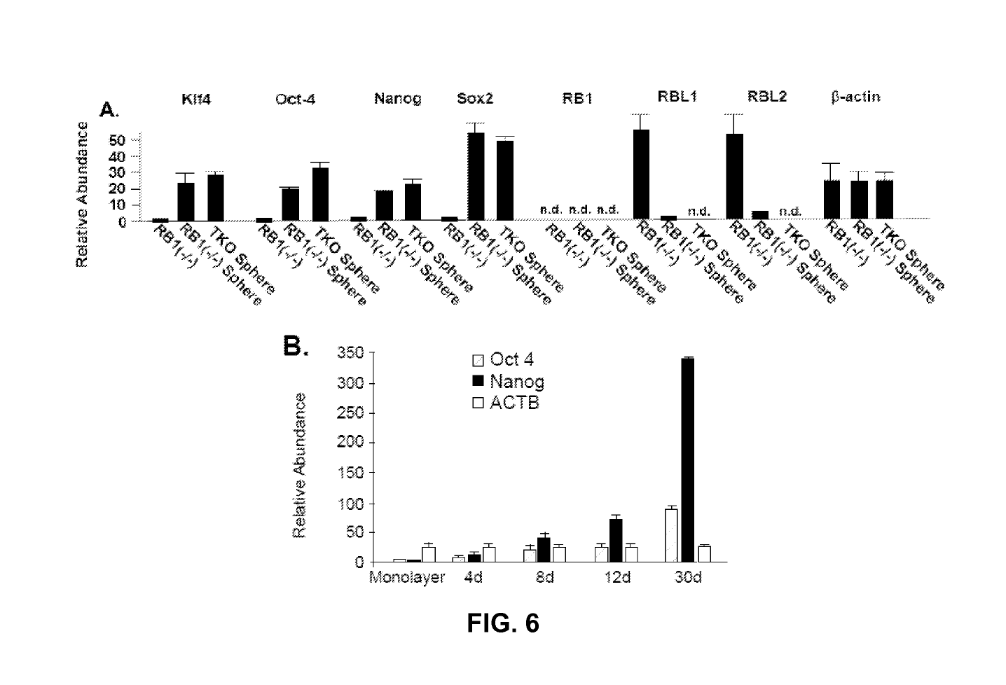

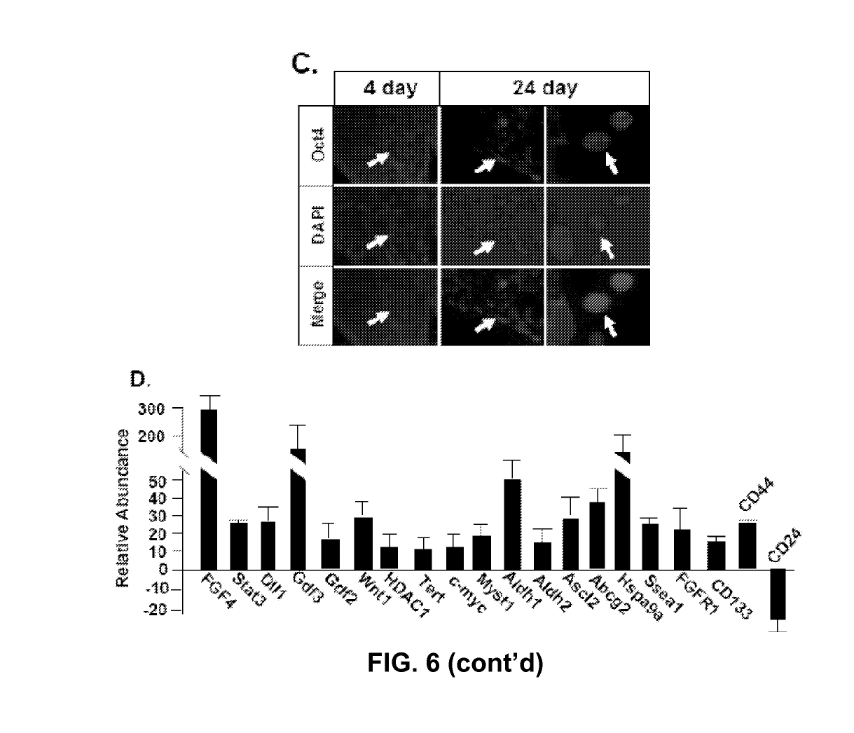

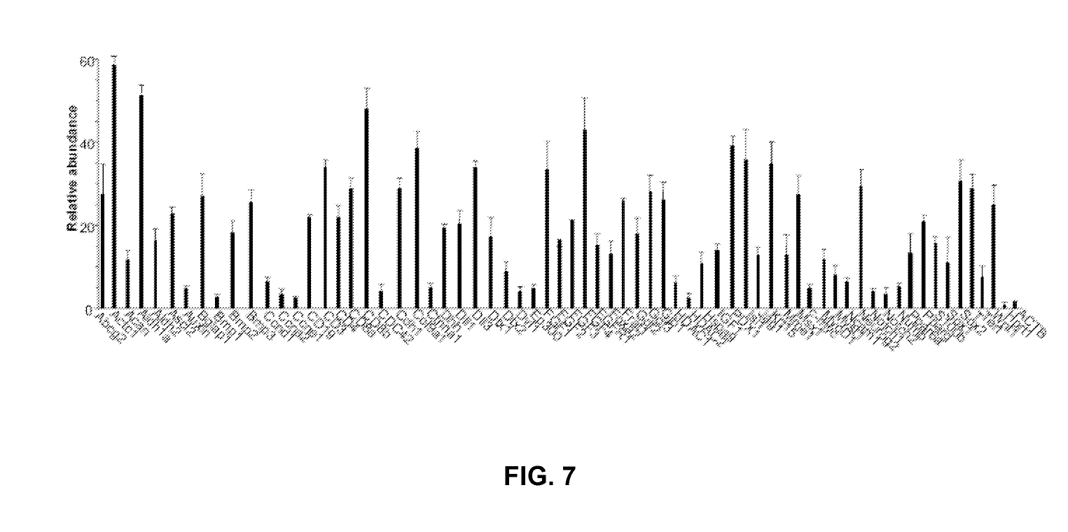

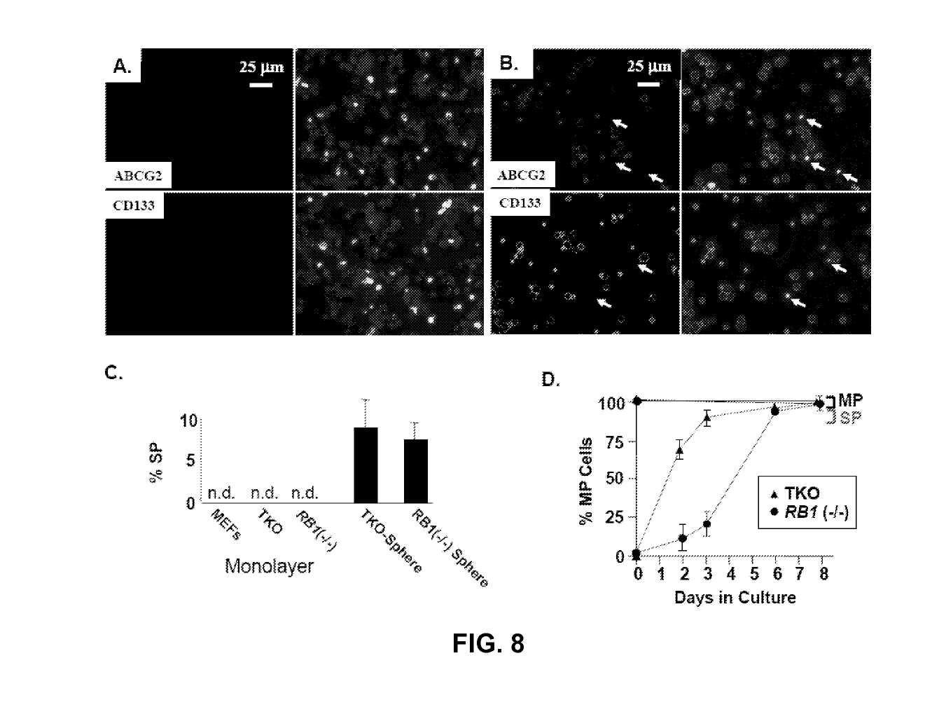

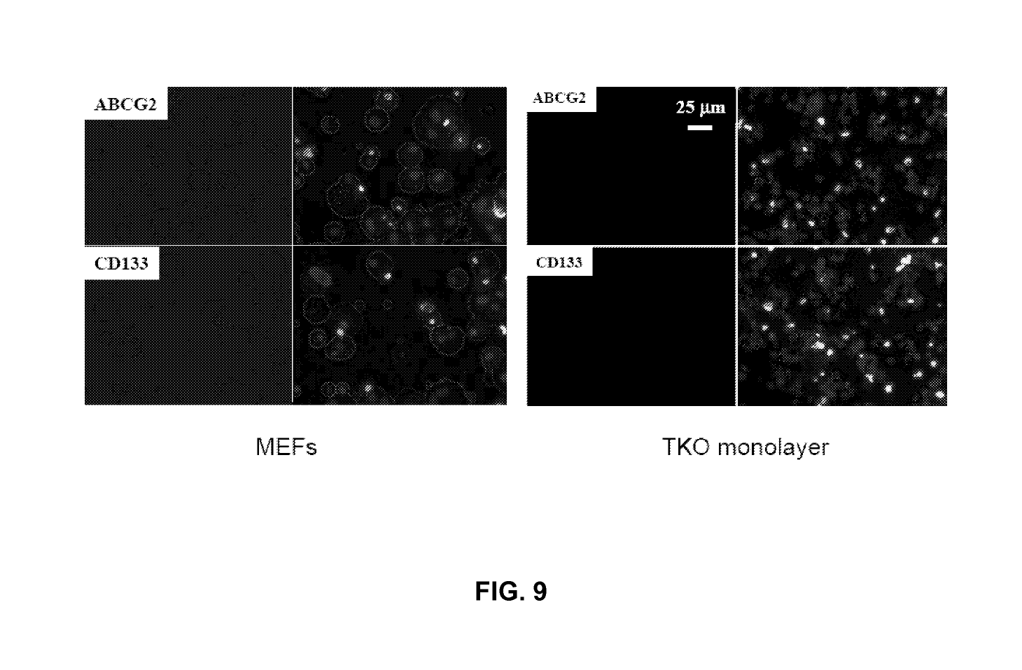

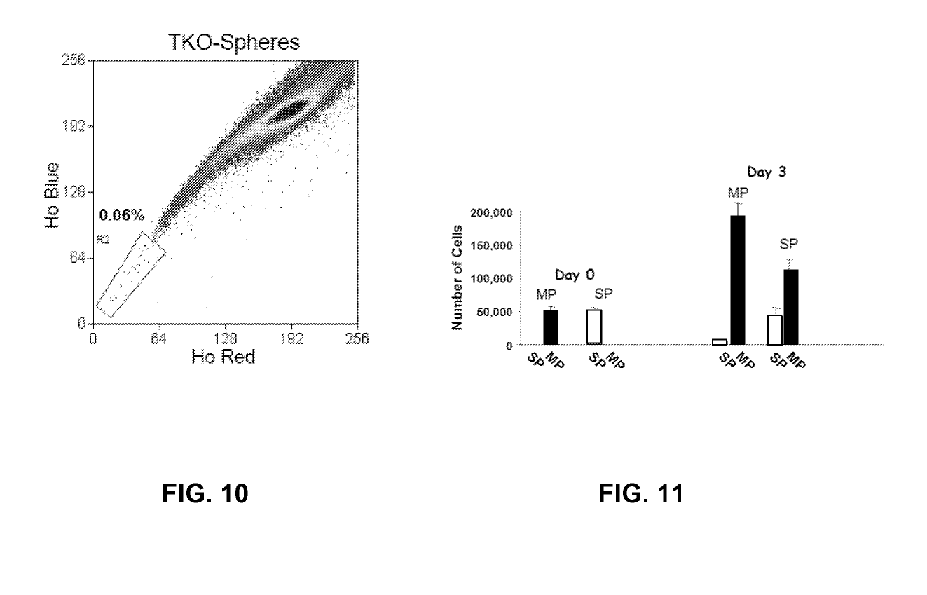

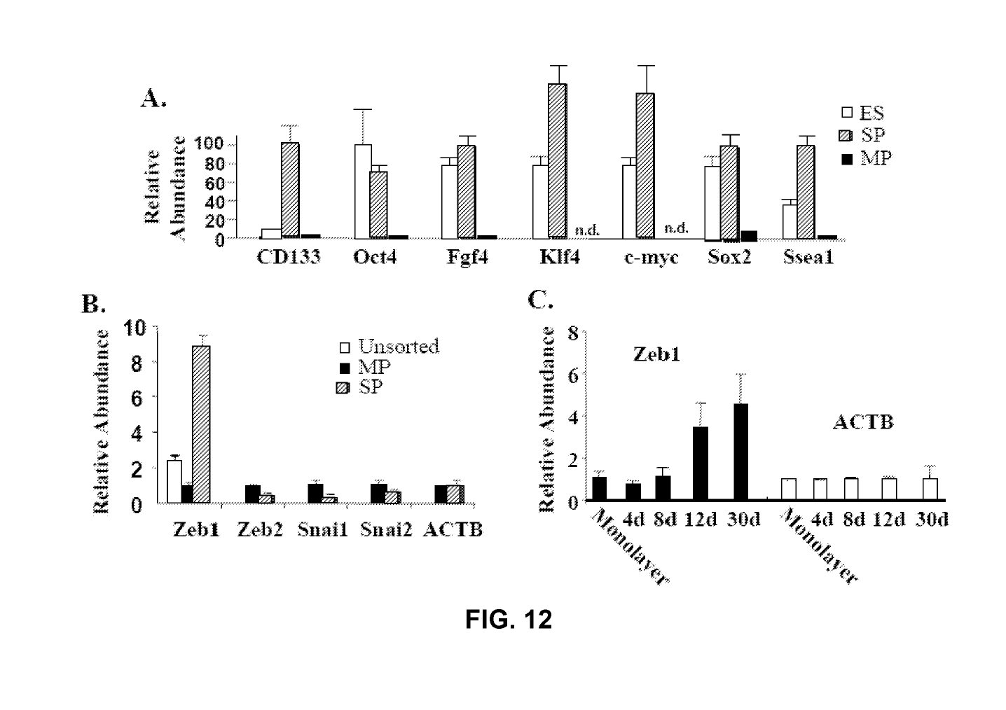

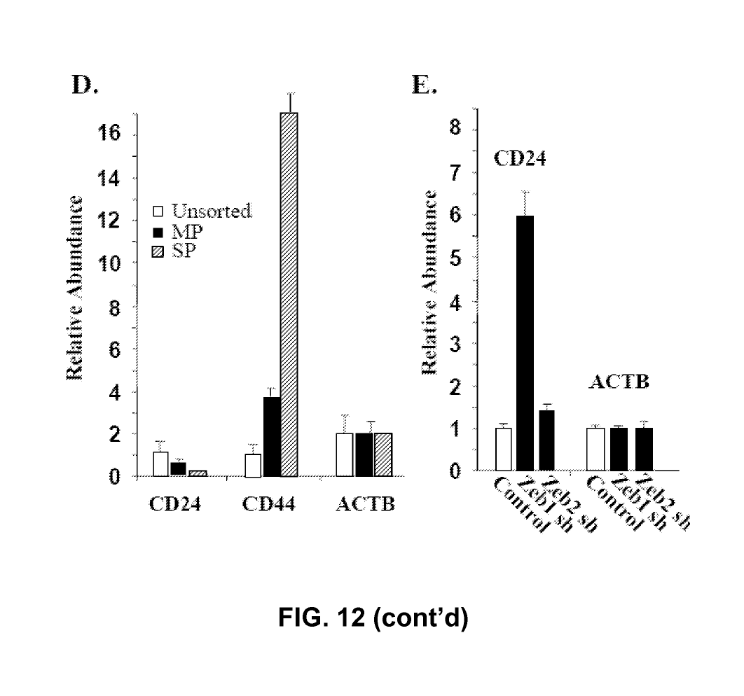

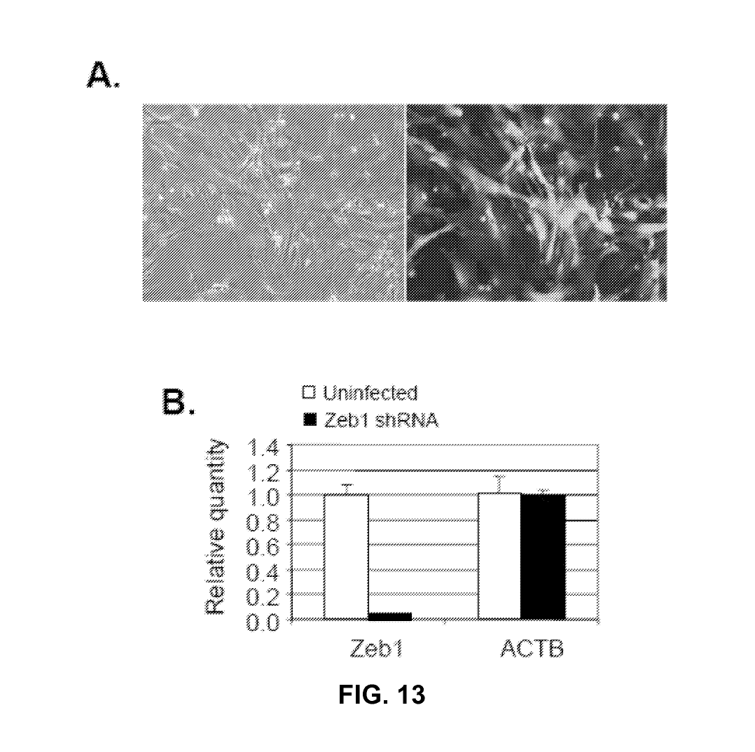

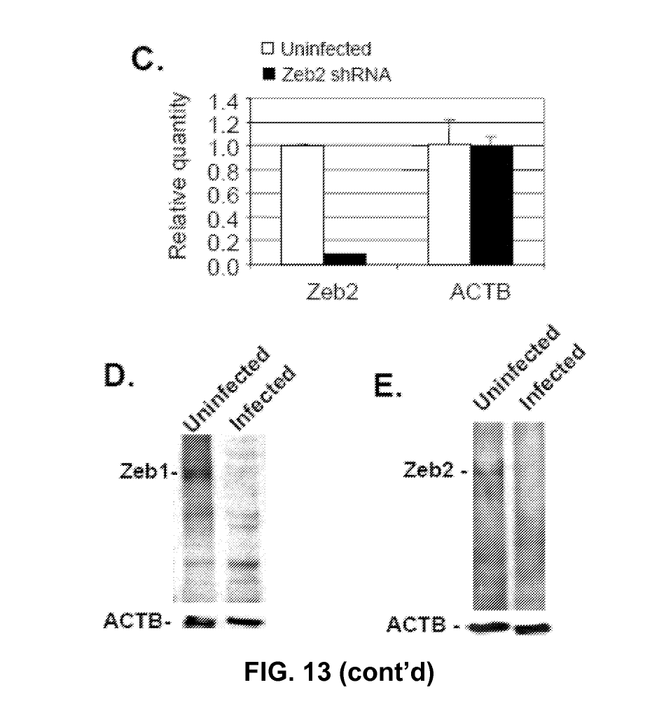

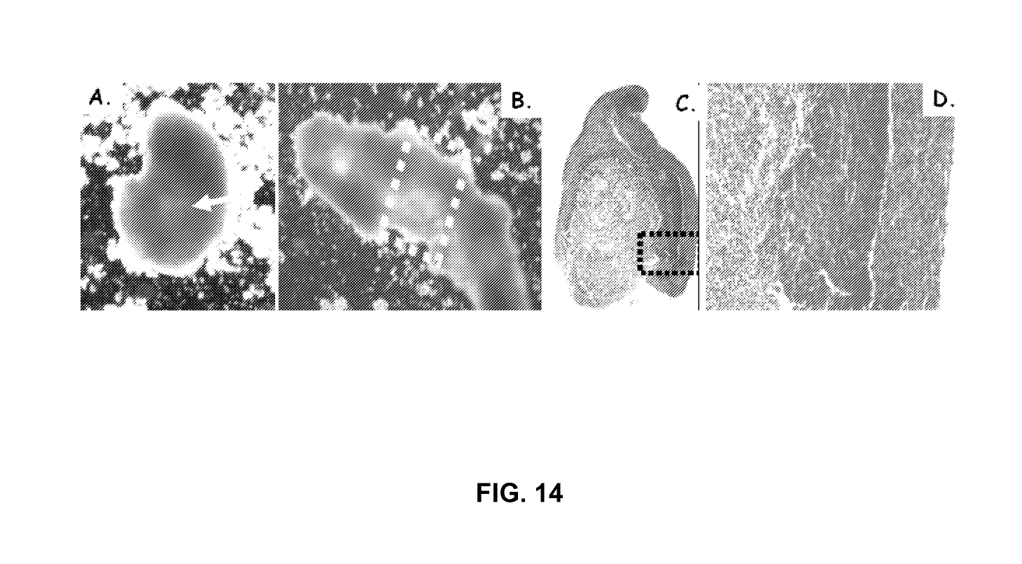

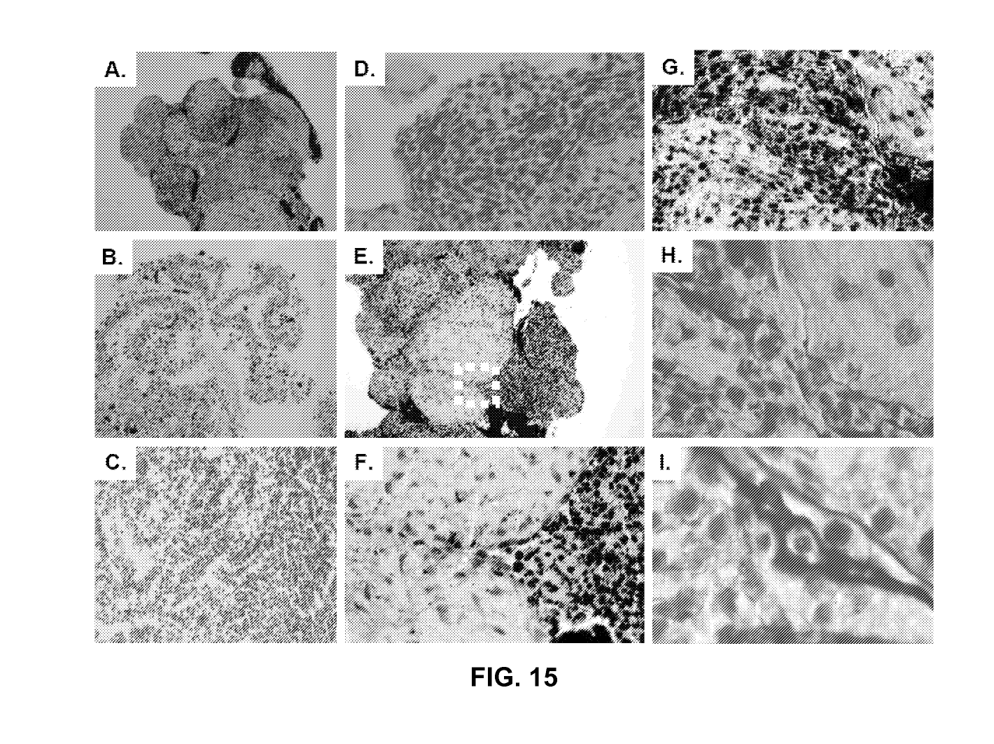

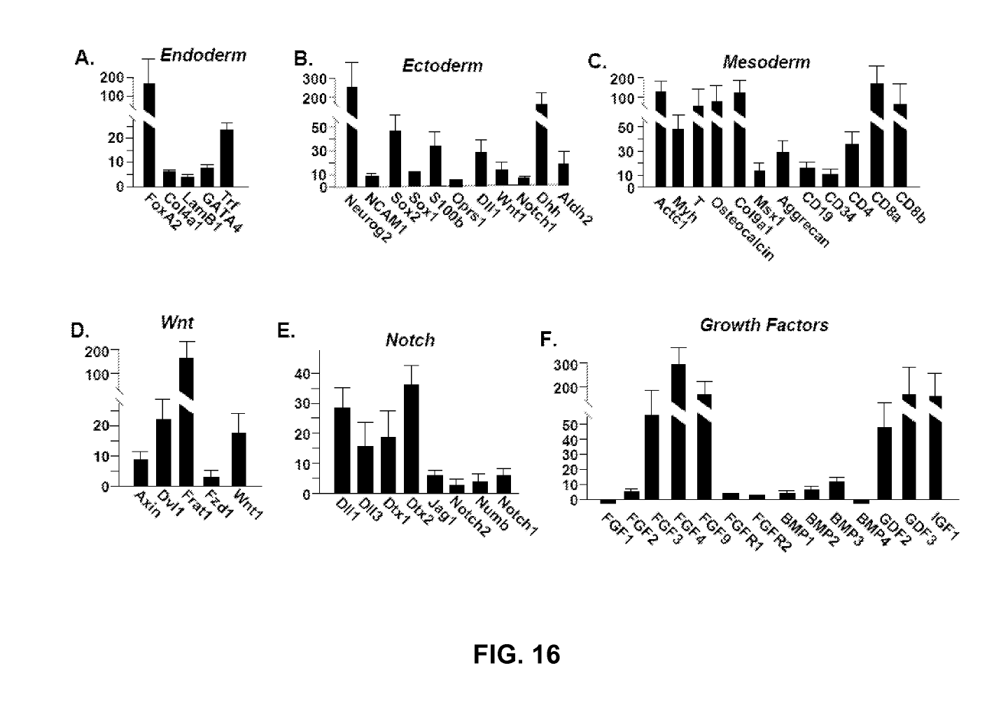

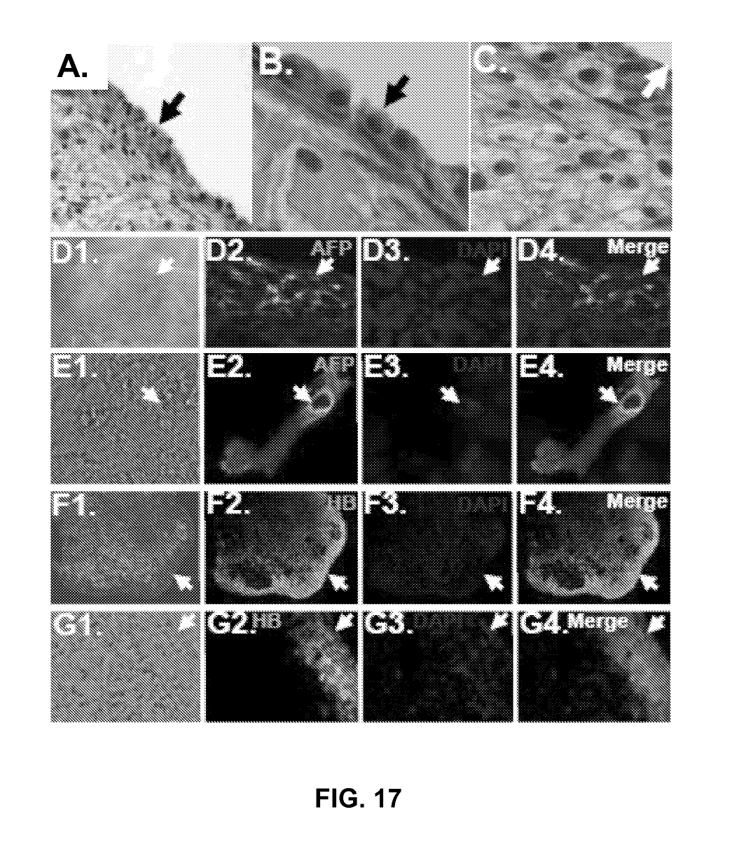

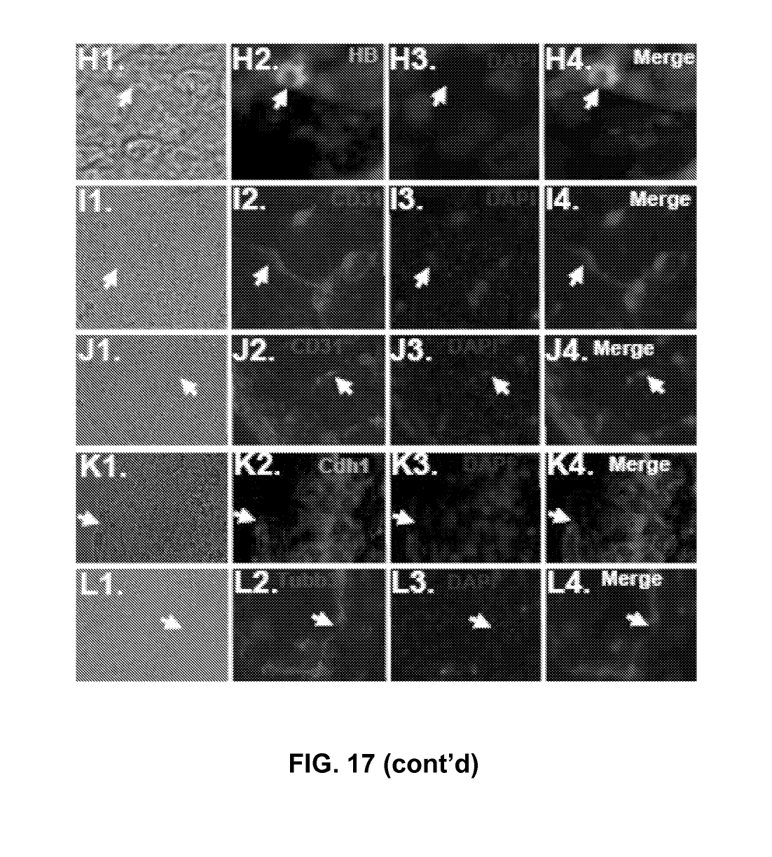

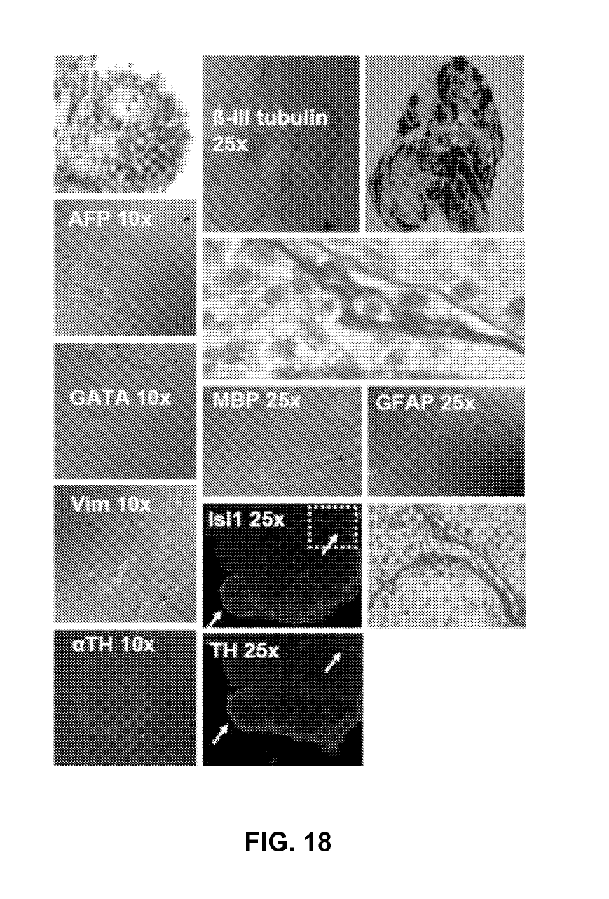

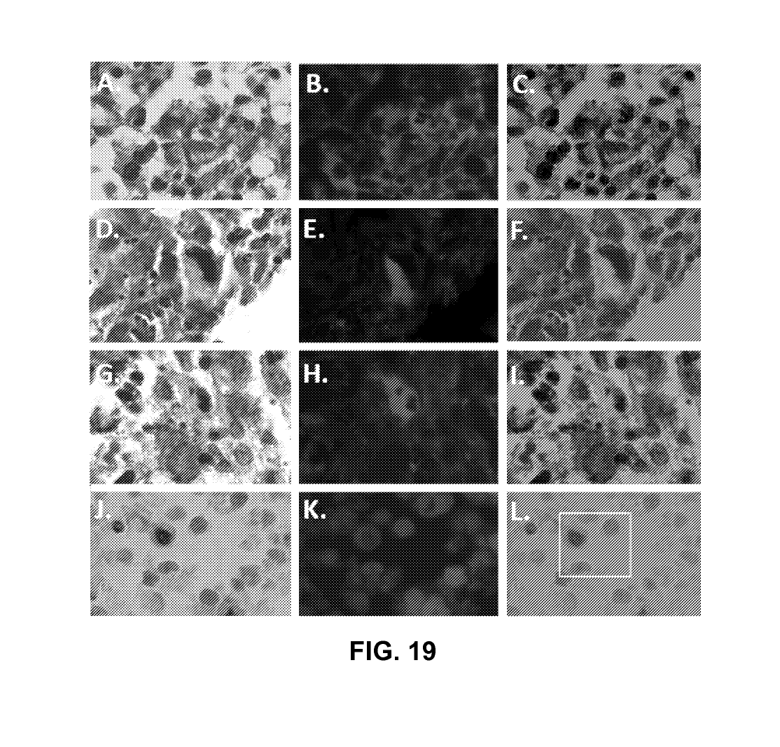

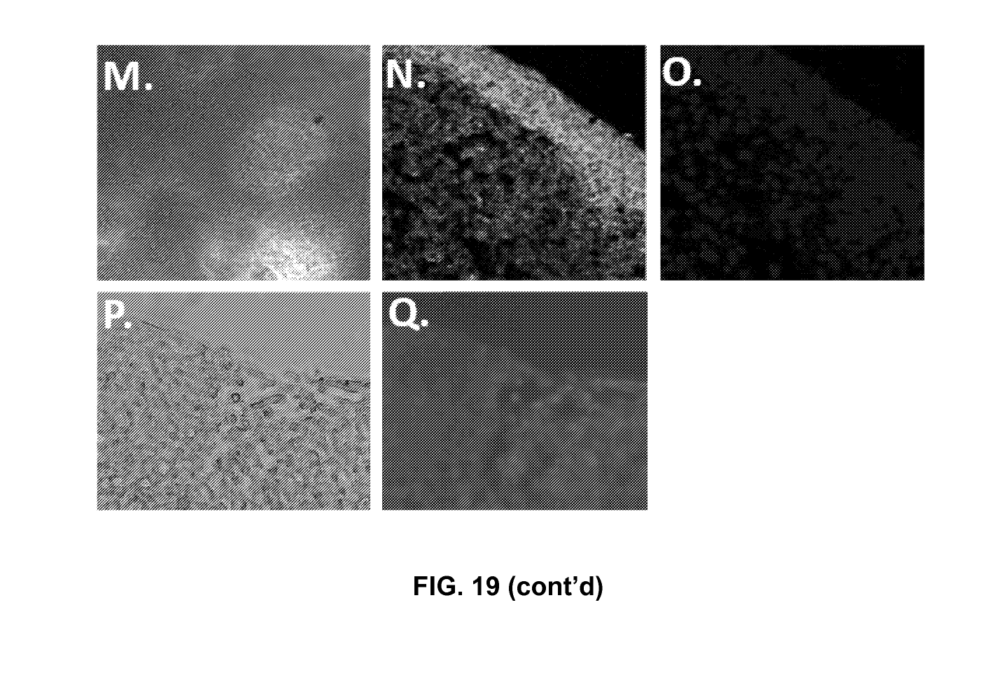

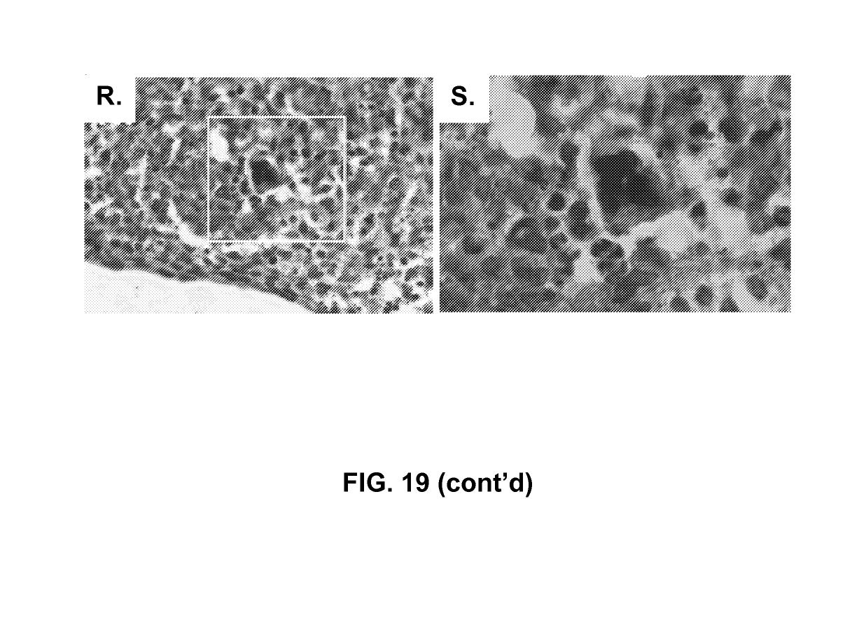

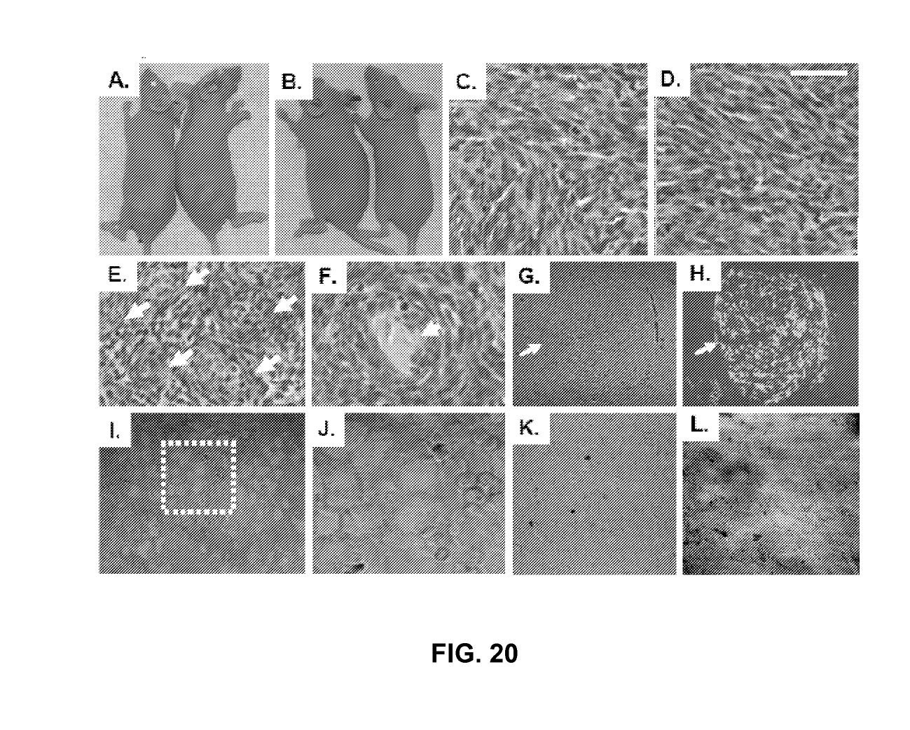

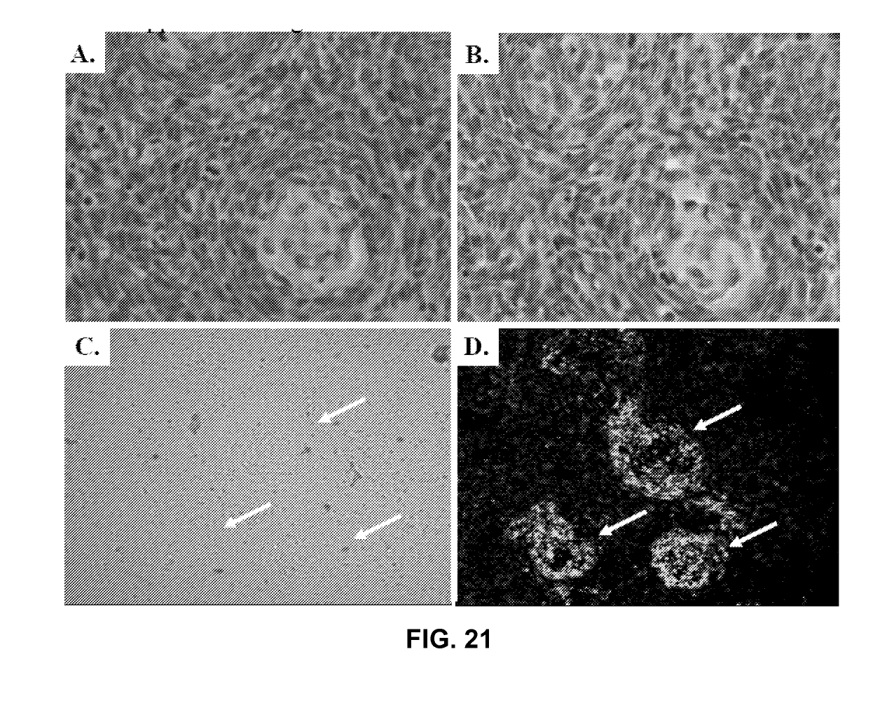

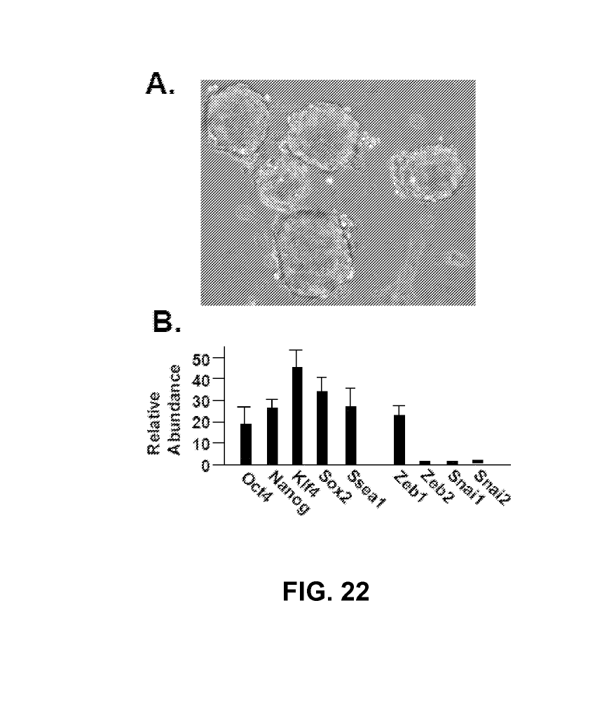

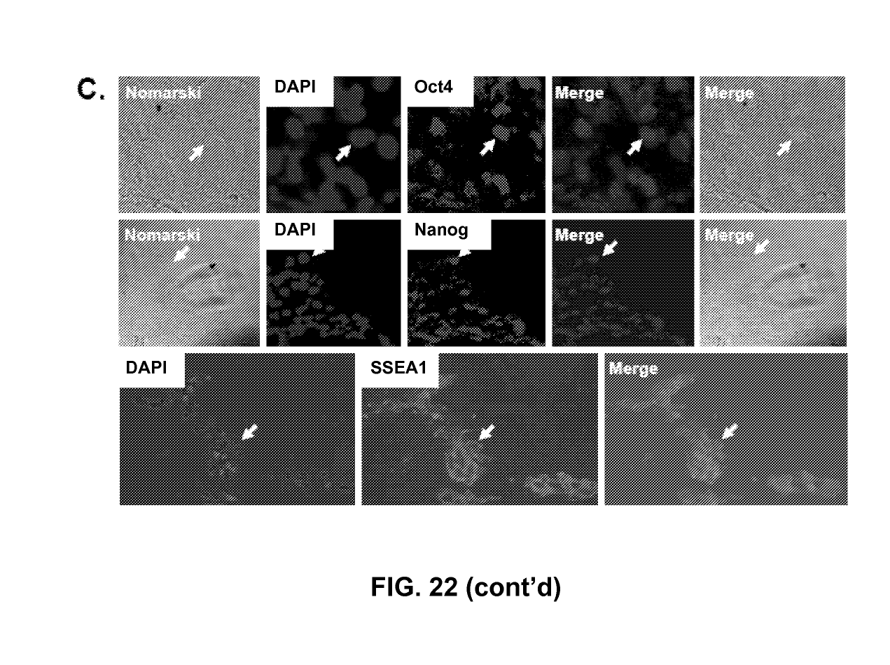

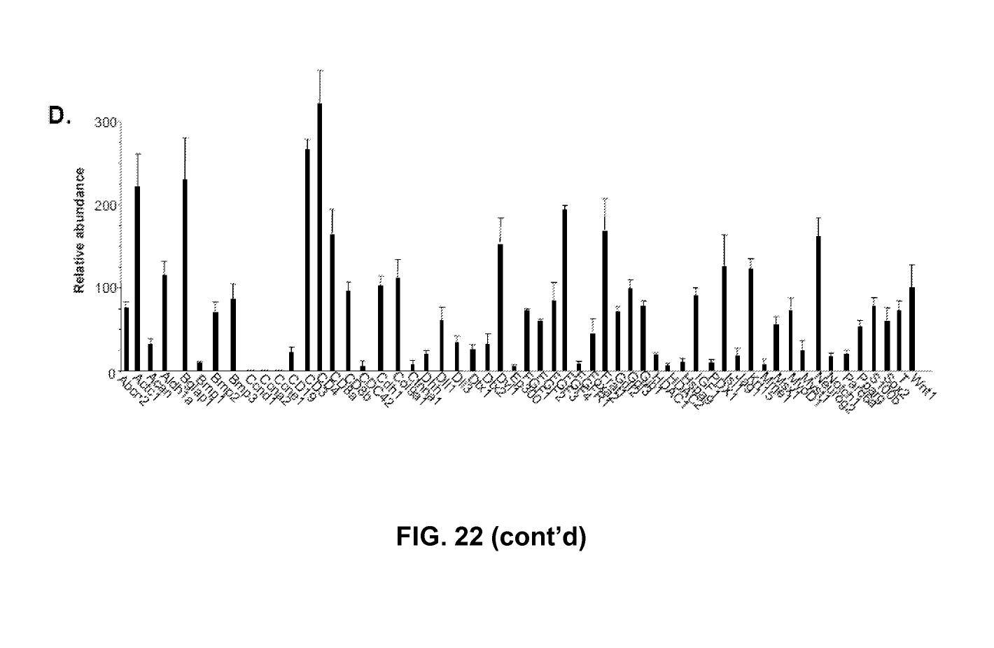

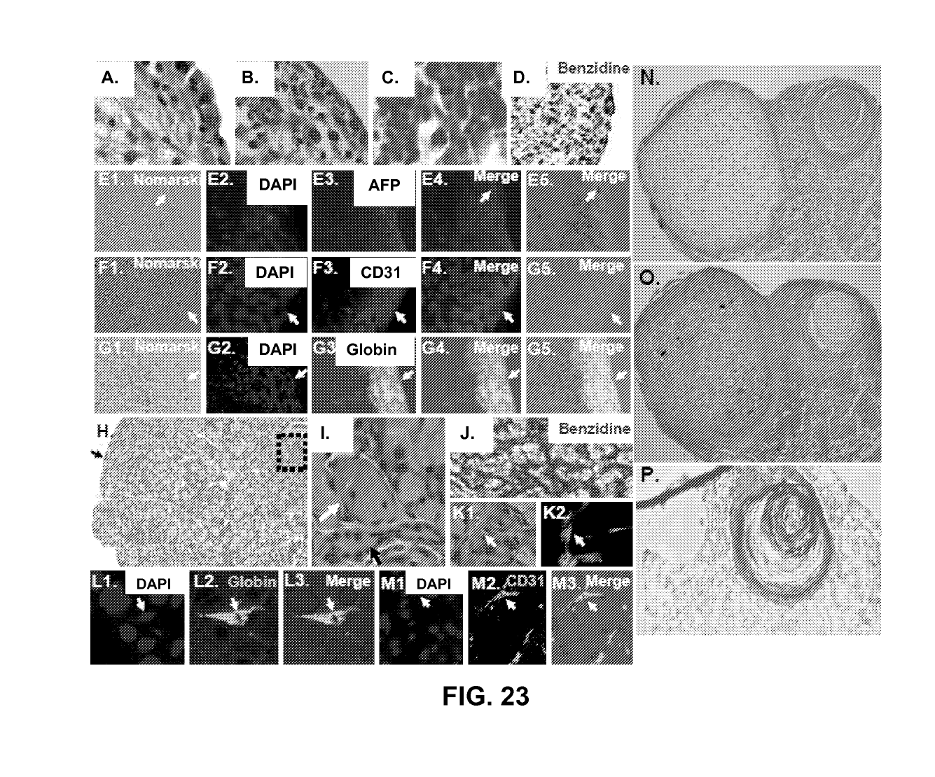

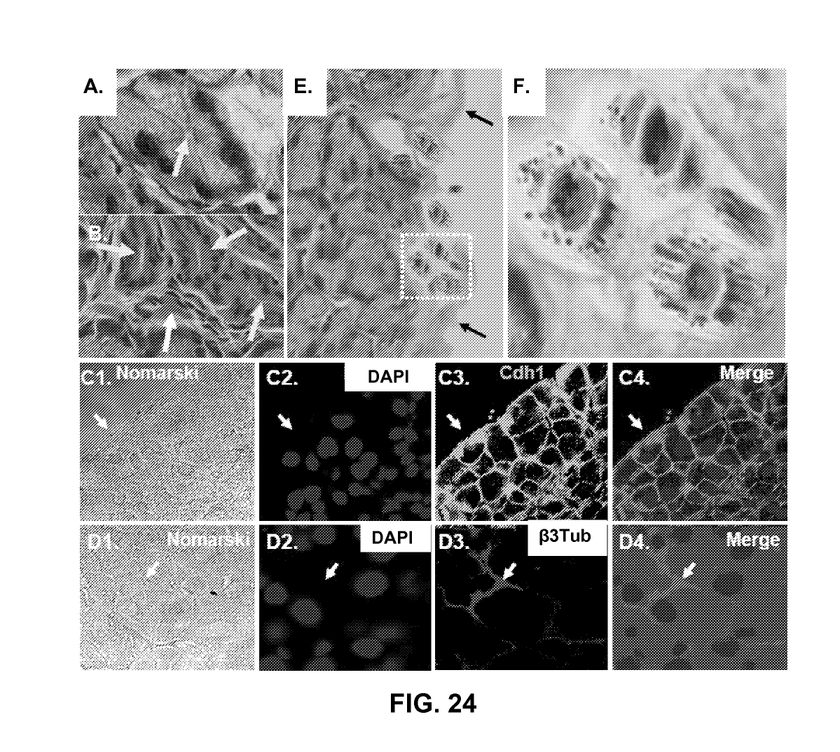

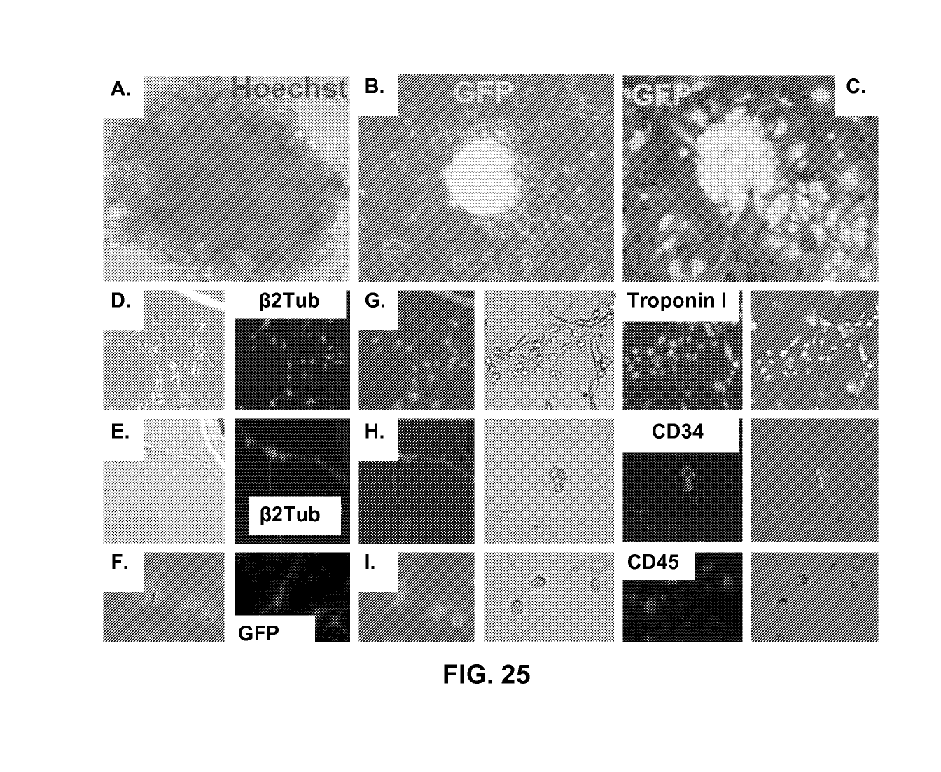

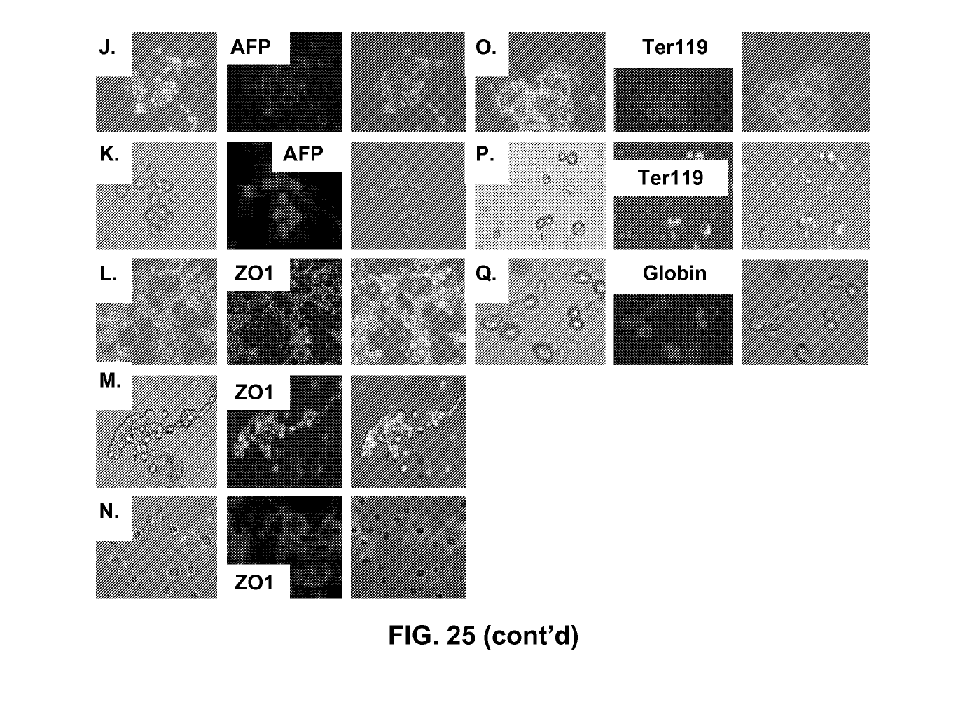

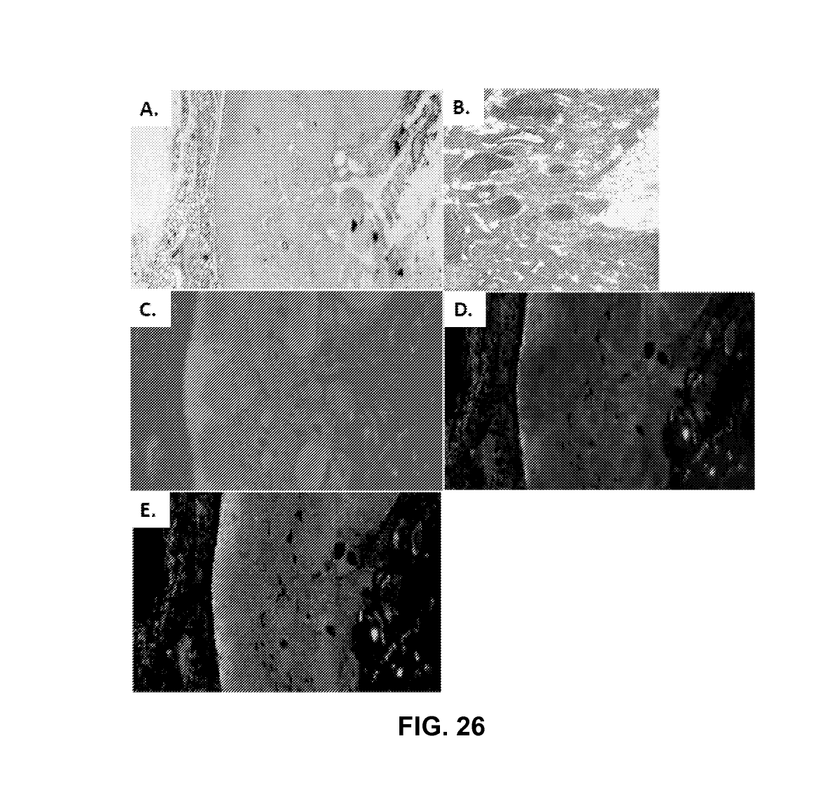

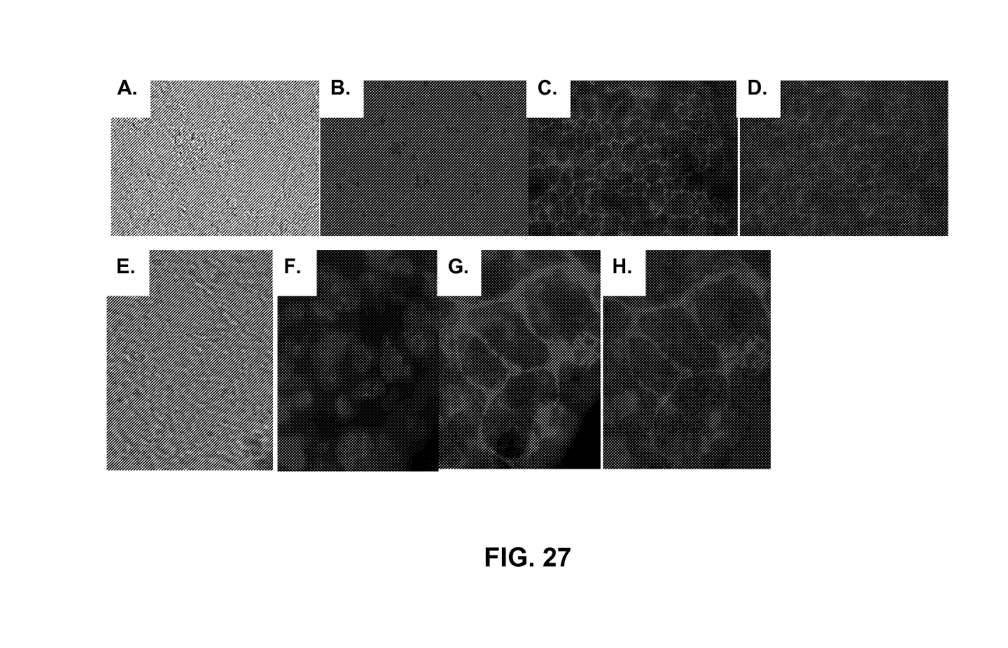

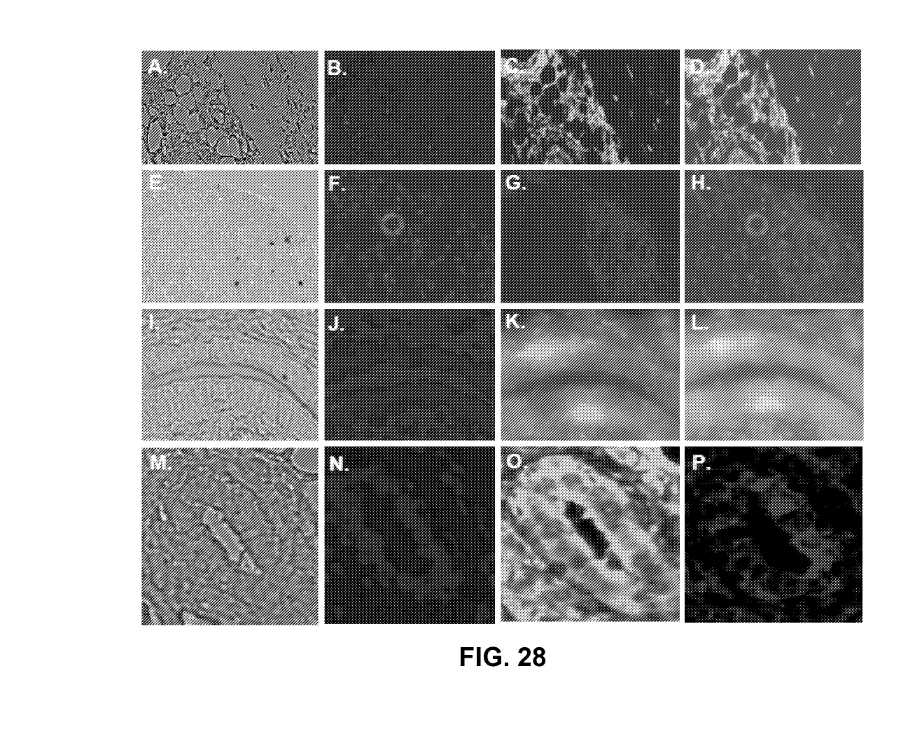

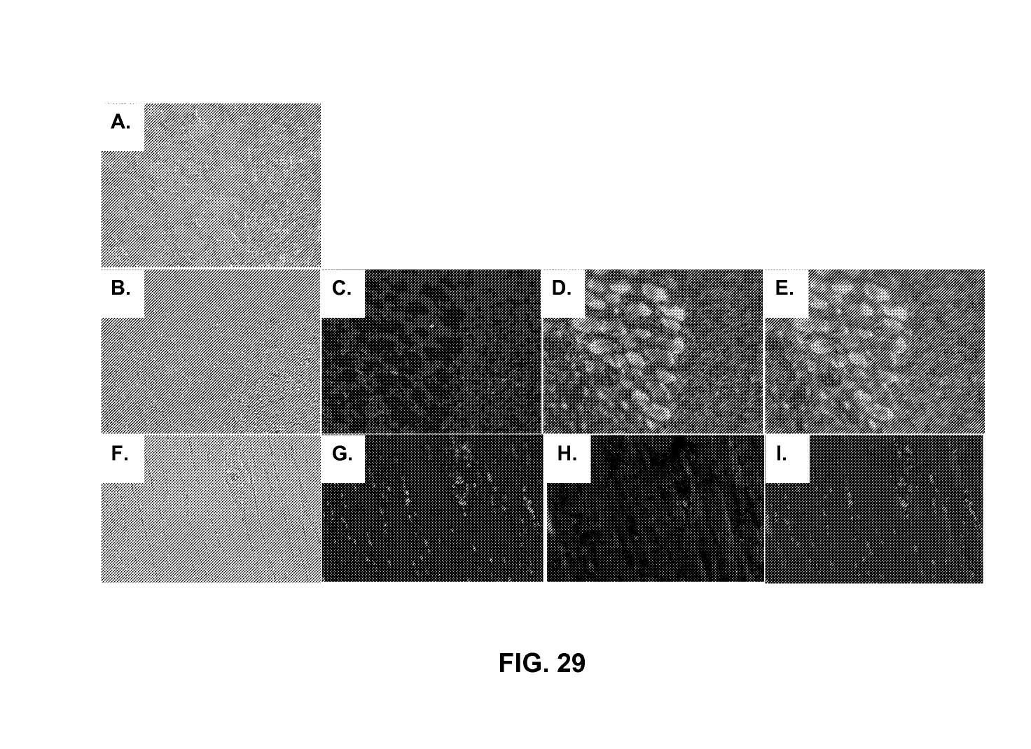

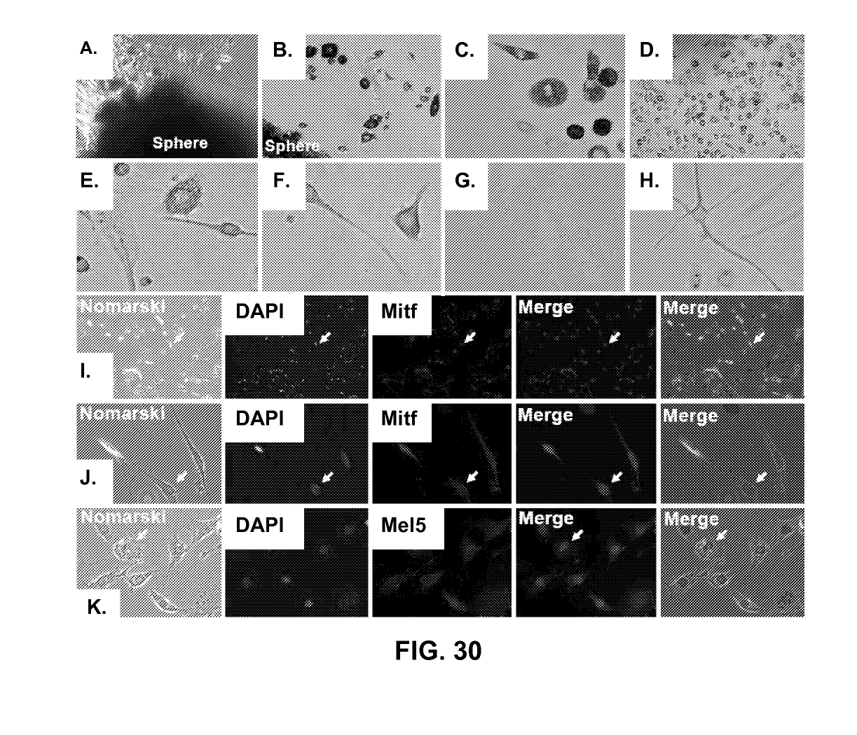

Consistent with their lack of cell-cell contact inhibition, once mouse embryo fibroblasts (MEFs) in which all three RB1 family members had been mutated (referred to herein as “triple knockouts” or “TKOs”) became confluent in culture, they began to stack up on one another leading to the generation of mounds of cells on the plates. See FIGS. 1A and 1B. Similar results were seen with cells at passages 4, 11, and 40, and with TKOs isolated from four different litters of mice. Subsequently, outgrowth of cells in these mounds led to detachment of the mounds from the culture plate and formation of spheres in suspension (see FIGS. 1C and 1D). This sphere formation was efficient, and with time, most TKO cells on the plate formed spheres. In contrast to TKOs, wild type MEFs, RB1^{-/-} MEFs, and RB1/RBL2^{-/-} MEFs remained contact inhibited, and thus did not form such mounds or spheres.

The TKO spheres visually resembled embryoid bodies that are produced when embryonic stem (ES) cells are placed in suspension culture (see FIGS. 1C and 1D; Desbaillets et al., 2000), and when transferred to non-adherent plates, these spheres could be maintained for at least two months in suspension. During this period, they increased in size and formed a central cavity (see FIG. 1E). When the spheres were transferred back to a tissue culture plate, they adhered to the plate and all of the cells within the spheres migrated back onto the plate to reform a monolayer (see FIGS. 1F and 1G). Surprisingly, none of the cells in these monolayers resembled the TKOs from which they were derived prior to sphere formation; they were smaller and morphologically heterogeneous (compare FIG. 1A to FIGS. 1H and 1I). The TKO sphere-derived cells retained this smaller size and distinct morphology as they were passaged in culture, demonstrating a stable morphological transition. The generation of cells with such morphology in TKOs that were maintained in subconfluent monolayer cultures was not observed, even after 40 passages.

When TKOs were trypsinized and suspended as single cells in culture, spheres did not form, and the single cells began to die after 24 hours in suspension (FIG. 2A). However, if TKOs present in confluent monolayers were scraped from the surface of a plate (i.e., without trypsinization), the cells formed spheres in suspension. Such spheres were indistinguishable in the experiments described herein below from mound-derived cells that spontaneously detached from confluent TKO cultures. Consistent with their lack of survival in suspension culture, individual trypsinized TKO did not form colonies in soft agar nor did they form tumors in nude mice (FIG. 3; see also below).

TKOs were then infected with an H-Ras^{V12}-expressing retrovirus as described in Telang et al., 2006. The H-Ras^{V12}-expressing retrovirus encoded the V12 oncogenic allele of H-ras. These new cells were referred to as TKO-Ras.

Western blot analyses of Ras expression and activity in MEFs, TKOs, and TKO-Ras cells are shown in FIGS. 4A and 4B. FIG. 4A is a digital image of a Western blot showing total Ras expression in TKOs and in TKO-Ras cells. The bottom panel of FIG. 4A shows β-actin expression, which was included as a loading control. FIG. 4B is a digital image of a Western blot showing activated Ras that was detected by binding to a fusion protein of Raf fused to glutathione-S-transferase (GST-Raf). The bottom panel of FIG. 4B shows a Western blot of input total Ras protein used for each assay. It was determined that not only did TKO-Ras cells have an increased level of Ras relative to TKOs (see FIG. 4A), an increased percentage of the Ras present was in an activated form (see FIG. 4B).

It was further determined that recombinant expression of activated H-Ras^{V12} in TKOs-Ras allowed for the survival and proliferation of trypsinized TKOs in suspension. Thus, whether TKO-Ras cells could form colonies in soft agar was also examined. Previously, Sage et al., 2000 reported that TKO-Ras cells could indeed form colonies in soft agar and tumors in nude mice (Sage et al., 2000), but Peeper et al., 2001 reported that H-Ras^{V12} expression did not allow for growth of TKOs in soft agar (Peeper et al., 2001).

Contrary to the results disclosed in Peeper et al., 2001, TKO-Ras cells did form colonies in soft agar and tumors in nude mice when 50,000 cells were injected (FIG. 3; see also below). Conceivably, the differential effects of H-Ras^{V12} in the TKO-Ras cells could be due to the levels of Ras expression in different cells, since three different H-Ras^{V12}-expressing cells were used in the studies.

Interestingly, TKO-Ras cells did not form spheres in suspension that resembled those formed by TKOs themselves (see FIG. 2B). Instead, single cells and small clusters of TKO-Ras cells began to appear in suspension after the TKO-Ras cells achieved confluence in culture. As with the trypsinized cells, these single cells and clusters survived and proliferated in suspension culture. When TKO-Ras cells in suspension were allowed to reattach to culture plates, they were visually indistinguishable from cells maintained in monolayer culture. Thus, the TKO-Ras cells in suspension did not undergo the morphological changes observed with TKO cells in spheres. Further, activated Ras allowed for survival and proliferation of single TKO cells in suspension. Formation of spheres allowed the TKOs to survive and proliferate in suspension in the absence of activated Ras.

### Example 2

### Sphere Formation in RB1^{-/-} MEFs Also Led to Survival in Suspension and Stable Morphological Changes

As noted above, persistence of contact inhibition in RB1^{-/-} MEFs (mediated by RBL1 and RBL2) prevented formation of mounds and in turn spheres in monolayer culture (FIG. 5A). However, scraping confluent monolayers of TKO cells and placing the cells in suspension culture led to formation of spheres with properties indistinguishable from those seen in spheres derived from mounds that spontaneously detached from confluent plates. Therefore, it was postulated that bypassing contact inhibition by scraping confluent RB1^{-/-} MEFs from plates and placing them in suspension culture might lead to sphere formation and generation of cells with a distinct morphology.

Indeed when RB1^{-/-} MEFs were scraped from the plates upon which they were growing, they formed spheres in suspension as efficiently as TKOs, the spheres were indistinguishable morphologically from those formed by TKOs, and they increased in size and remained viable for at least two months in culture (FIG. 5B). As with TKO spheres, RB1^{-/-} MEF spheres in suspension culture on non-adherent plates reattached when transferred to tissue culture plates, and all cells in the spheres migrated back onto the plate to reform a monolayer (FIG. 5C). As with TKO-sphere-derived cells, RB1^{-/-} cells in these monolayers were small, morphologically diverse, and distinct from the original RB1^{-/-} MEFs (see FIG. 5D). Real Time PCR demonstrated that mRNAs for RBL1 and RBL2 were downregulated in the RB1^{-/-} spheres, potentially accounting for the loss of contact inhibition in the spheres (see FIG. 6A).

### Example 3

### Sphere Formation in TKOs and RB1^{-/-} MEFs Led to Expression of ES Cell Genes

Real Time PCR was used to examine gene expression in TKOs and RB1^{-/-} MEFs prior to and following sphere formation. Induction of classic stem cell marker mRNAs was observed in cells derived from spheres after two weeks in suspension culture. These mRNAs included Oct4, Nanog, Sox2, and Klf4 (see FIG. 6A). Expression of both Oct4 and Nanog mRNA increased during a time course of RB1^{-/-} MEF sphere formation in suspension culture (FIG. 6B).

To confirm Oct4 protein expression, spheres were immunostained for Oct4. After 4 days in suspension, only low level cytoplasmic staining for Oct4 was observed (FIG. 6C). Even though this cytoplasmic staining was dependent upon the primary antibody, little or no Oct4 mRNA was detected at this time (FIG. 6B). Thus, this cytoplasmic immunostaining might have been non-specific, as has been reported previously for Oct4 (Lengner et al., 2007).

After 8 days in suspension culture, strong nuclear immunostaining for Oct4 became evident in clusters of cells present in the spheres, and this correlated with the appearance of Oct4 mRNA by Real Time PCR. The number of cells showing nuclear Oct4 immunostaining increased at 24 days, and during this period there was a corresponding increase in the level of Oct4 mRNA (FIGS. 6B and 6C).

Nanog is a downstream target of Oct4 and thus its expression can be viewed as a functional readout of Oct4 activity. The level of Nanog mRNA paralleled that of Oct4 during this time course of sphere culture (FIG. 6B). In addition to these stem cell-specific genes, upregulation of other genes associated with stem cells was observed in both TKO and RB1^{-/-} MEF spheres (FIG. 6D; FIG. 7). For example, expression of CD44 and CD133 was induced, and CD24 expression was downregulated (see FIG. 6D).

### Example 4

### A Subset of Cells with Properties of a Side Population (SP) was Generated in TKO and RB1^{-/-} MEF Spheres

Wild type MEFs, TKOs maintained as subconfluent monolayers, and TKOs derived from spheres were tested for Hoechst dye exclusion and cell surface expression of Abcg2 and CD133. MEFs and TKOs maintained as subconfluent monolayers did not exclude Hoechst dye or express Abcg2 or CD133 on their surfaces (FIGS. 8A and 8C; FIG. 9). However, about 10% of sphere-derived TKOs were Hoechst⁻/Abcg2⁺/CD133⁻ (see FIGS. 8B and 8C). Notably, these Hoechst⁻/Abcg2⁻/CD133⁺ cells were much smaller (about 5 microns in diameter) than the main population (MP), which included Hoechst⁺/Abcg2⁻/CD133⁻ cells that were typically greater than 10 microns in diameter. See FIG. 10.

RB1^{-/-} cells were then examined for SP properties including exclusion of Hoechst dye; cell surface expression of Abcg2 and CD133; small size (e.g., about 5-7 microns in diameter); and expression of Klf4, Oct4, Sox2, and c-myc in levels similar to those seen in ES cells. Additional properties identified for these cells included an ability to divide asymmetrically to yield additional SP cells and MP cells, and ability of a low number (as few as 100 cells) to generate tumors in nude mice. MP cells lacked these properties. Also unlike MP cells, the tumors formed with SP cells contained cancer cells as well as differentiated cells expressing the neuronal marker beta3 tubulin. MP tumors did not contain differentiated cells (see below).

As with wild type MEFs, the RB1^{-/-} MEFs in monolayer culture did not display SP properties (e.g., exclusion of Hoechst dye and expression of Abcg2 and CD133; see FIG. 8C); however, cells derived from RB1^{-/-} MEF spheres showed a similar SP population to TKOs (FIG. 8C).

The sorted MP cells were analyzed. These cells were proliferative, but they did not divide asymmetrically to give rise to SP cells (FIG. 8D). However, it is of note that while the sorted MP cells were originally devoid of SP cells, a small number of SP cells appeared in the dividing MP culture (˜1%), and this number remained relatively constant in the proliferating MP population for at least one month (FIG. 11). Taken together, it appeared that SP cells from both TKO and RB1^{-/-} spheres could give rise to MP cells via asymmetric division, and that the MP cells in turn could divide symmetrically to increase their number in the population (although there was a low level of SP cell generation in the MP).

### Example 5

### The Hoechst⁻/Abcg2⁺/CD133⁺ SP Cells Express Stem Cell Markers

Gene expression in sorted SP and MP populations of cells derived from spheres was compared to that in embryonic stem (ES) cells using Real Time PCR. The SP cells from spheres expressed mRNAs for stem cell markers in levels similar to those seen in ES cells (FIG. 12A). These markers included Oct4, Sox2, c-myc, and Klf4, for which retroviral re-expression had been shown to be sufficient for reprogramming of MEFs to pluripotency (Takahashi & Yamanaka, 2006; Okita et al., 2007; Wernig et al., 2007; Jaenisch & Young, 2008). Conversely, there was little expression of the stem cell mRNAs in the MP cells. These results suggested that the Oct4⁺ and Nanog⁺ cells observed in spheres corresponded to SP cells, and that as the SP cells divided stem cell genes were downregulated and/or silenced in daughter MP cells. As noted above, TKO-Ras cells did not form spheres in suspension nor did they express significant levels of Oct4, Klf4, or Nanog mRNAs.

### Example 6

### Zeb1 mRNA is Induced in SP Cells and is Associated with a CD44 High/CD24 Low mRNA Expression Pattern

Overexpression of E-box binding transcriptional repressors, including Snai-1, Snai-2, twist, Zeb1, and Zeb2, typically leads to repression of E-cadherin and epithelial-mesenchymal transition (EMT), and Snail repression of E-cadherin and EMT appears to be mediated at least in part through induction of Zeb1 and Zeb2 (Peinado et al., 2007). Recent studies have demonstrated that overexpression of these EMT factors can also trigger a CD44^{high}/CD24^{low} pattern on epithelial cells, which is associated with acquisition of stem cell and cancer stem cell properties by somatic cells (Mani et al., 2008). Therefore, whether expression of these EMT transcription factors was induced in the sphere-derived SP cells was tested.

Using Real Time PCR, it was determined that Zeb1, but not Zeb2, snai1, or snai2, mRNA was induced in SP cells compared to MP cells (FIG. 12B), and that Zeb1 mRNA increased in a time course of sphere formation in RB1^{-/-} MEFs similar to that seen with Oct4 and Nanog mRNA (FIGS. 6B and 12C).

Next, whether overexpression of Zeb1 mRNA coincided with induction of CD44 mRNA and downregulation of CD24 mRNA in SP cells was tested. Indeed, CD44 mRNA was induced in SP cells, whereas CD24 mRNA was diminished (FIG. 12D). In addition to this CD44^{high}/CD24^{low} mRNA pattern in the SP cells, it was observed that CD133 mRNA and protein was also induced in the SP cells along with Zeb1 mRNA (FIG. 12A).

Both Zeb1 and Zeb2 are expressed in wild type MEFs (Liu et al., 2007a; Liu et al., 2008), and while CD44 mRNA was not detected in these cells, CD24 mRNA was present (FIG. 12E). Lentiviral shRNA constructs were employed to knock down Zeb1 and Zeb2 expression in these cells (FIGS. 13A-13E) to determine whether either of these EMT transcription factors might be important in maintaining repression of CD24.

For this purpose, MEFs were infected with a GFP-expressing lentiviral vector. FIG. 13A is a set of photomicrographs showing an example of GFP expression in such MEFs. The left panel is a bright field photograph, and the left panel is a fluorescence micrograph showing the expression of GFP in the infected MEFs.

Lentiviral vectors that encoded shRNAs directed against Zeb1 and Zeb2 were then employed as described hereinabove (see “Lentivirus shRNA Methods”). FIGS. 13B and 13C are bar graphs showing RNA levels of Zeb1 and Zeb2 in uninfected vs. shRNA-containing cells, respectively, determined by Real Time PCR. β-actin (ACTB) expression levels were also tested as a negative control. As can be seen, both the knockdowns resulted in greater than 90% reductions in RNA for Zeb1 (FIG. 13B) and Zeb2 (FIG. 13C). The reduction was also observed at the protein level (see FIGS. 13D and 13E, which are digital images of Western blots for Zeb1 and Zeb2, respectively, in uninfected and infected cells).

Expression of CD24 in knockdown cells was also examined by Real Time PCR. It was determined that knockdown of Zeb2 had little effect on the level of CD24 mRNA. However, CD24 mRNA was significantly induced with Zeb1 knockdown. These results provided evidence that the normal level of Zeb1 in the cells played a role in repressing CD24.

### Example 7

### RB1^{-/-} and TKO MEF Spheres Express Markers of All Three Embryonic Layers

The appearance of SP cells expressing stem cell markers in TKO and RB1^{-/-} MEF spheres, together with the diverse morphology seen in cells derived from these spheres (see FIGS. 1H and 1I; FIGS. 5, 14, and 15), led to an investigation of whether there was evidence of differentiation in the spheres (e.g., analogous to differentiation seen when embryonic stem cells form embryoid bodies). Real Time PCR was employed to analyze mRNA expression in spheres and in cells which had been allowed to migrate from spheres and reform monolayers on tissue culture plates. Results were similar with the spheres and the sphere-derived monolayers.

mRNA expression in the sphere-derived cells was also compared to that in cells maintained as subconfluent monolayers. The results are summarized in Table 5.

TABLE 5
Real Time PCR to Compare mRNA Expression in Monolayer
Culture: MEFs vs. TKO¹

| | | | | | |
|---|---|---|---|---|---|
| Symbol | AVG | STD | Symbol | AVG | STD |

1. Hematopoietic

| | | | | | |
|---|---|---|---|---|---|
| CD19 | 2.162756 | 0.918958 | CD8b1 | 3.936995 | 2.663557 |
| CD3d | 1.617454 | 1.223371 | Cxcl12 | 1.822446 | 0.073269 |
| CD4 | 3.749245 | 1.782565 | CD34 | 0.157265 | 0.043373 |
| CD8a | 5.686071 | 4.412893 | | | |

2. Notch signaling

| | | | | | |
|---|---|---|---|---|---|
| Dll1 | 1.148384 | 0.601116 | Jag1 | 2.564684 | 1.33494 |
| Dll3 | 1.113073 | 0.726302 | Notch2 | 0.679858 | 0.125039 |
| Dtx1 | 1.402929 | 1.070028 | Numb | 1.874094 | 0.449959 |
| Dtx2 | 2.152268 | 0.552309 | Notch1 | 1.539392 | 0.374914 |

3. Wnt signaling

| | | | | | |
|---|---|---|---|---|---|
| Axin1 | 1.201534 | 0.376246 | Fzd1 | 0.281172 | 0.070987 |
| Dvl1 | 3.235461 | 1.582196 | Wnt1 | 1.307538 | 1.156752 |
| Frat1 | 2.552954 | 1.296211 | | | |

4. Cell cycle

| | | | | | |
|---|---|---|---|---|---|
| Ccna2 | 0.405613 | 0.07395 | Ccne1 | 0.431615 | 0.031129 |
| Ccnd1 | 0.851618 | 0.132826 | Cdc2a | 0.531838 | 0.085725 |
| Ccnd2 | 6.150291 | 0.628415 | | | |

5. FGF regulation

| | | | | | |
|---|---|---|---|---|---|
| Fgf1 | 1.356579 | 0.432323 | Fgf4 | 3.907379 | 1.139585 |
| Fgf2 | 4.165012 | 0.515002 | Fgfr1 | 2.191219 | 0.124001 |
| Fgf3 | 1.478631 | 0.482986 | Fgfr2 | 0.578845 | 0.034025 |

6. BMP signaling

| | | | | | |
|---|---|---|---|---|---|
| Bmp1 | 2.157023 | 0.4534 | Gdf2 | 1.939791 | 0.274414 |
| Bmp2 | 2.159411 | 0.813333 | Gdf3 | 3.459464 | 0.481626 |
| Bmp3 | 1.743361 | 0.796377 | BMP4 | 0.825059 | 0.654531 |

7. Stem cell

| | | | | | |
|---|---|---|---|---|---|
| Myst1 | 1.299416 | 0.236055 | Gdf3 | 3.459464 | 0.481626 |
| Aldh1a1 | 13.33841 | 5.658154 | Hspa9a | 1.562171 | 0.12653 |
| Aldh2 | 1.705199 | 0.8791 | Krt1-15 | 0.979351 | 0.41743 |
| Cd44 | 1.473242 | 0.189387 | Prom1 | 0.663089 | 0.093934 |
| Neurog2 | 2.124203 | 1.844036 | Oct4 | n.d. | n.d. |
| Sox2 | 0.858702 | 0.576787 | CD34 | 0.157265 | 0.043373 |
| Dll1 | 1.148384 | 0.601116 | Nanog | 3.883355 | 3.539828 |
| Fgf3 | 1.478631 | 0.482986 | Stat3 | 1.771547 | 0.008683 |
| Fgf4 | 3.907379 | 1.139585 | | | |

8. Endoderm

| | | | | | |
|---|---|---|---|---|---|
| Foxa2 | 2.476501 | 1.109203 | GATA4 | 1.554909 | 0.280444 |
| Aldob | 1.294869 | 0.11409 | LAMB1 | 3.063086 | 0.359485 |
| Col4 | 6.085709 | 0.208754 | Trf | n.d. | n.d. |

9. Mesoderm

| | | | | | |
|---|---|---|---|---|---|
| Actc1 | 4.218635 | 0.742679 | Msx1 | 1.261426 | 0.789689 |
| Bglap1 | 1.251945 | 0.336007 | Col9a1 | 3.245166 | 1.36648 |
| T | 1.434407 | 1.020334 | Col4 | 6.085709 | 0.208754 |
| Agc1 | 2.245066 | 0.659756 | Myh2 | 3.287027 | 0.449688 |
| Cd19 | 2.162756 | 0.918958 | | | |

10. Neural/Ectoderm

| | | | | | |
|---|---|---|---|---|---|
| Adar | 1.693513 | 0.281798 | Oprs1 | 0.782157 | 0.024446 |
| Agc1 | 2.245066 | 0.659756 | S100b | 1.553241 | 0.260488 |
| Aldh2 | 1.705199 | 0.8791 | Sox1 | 1.619727 | 0.994576 |
| Cd44 | 1.473242 | 0.189387 | Sox2 | 0.858702 | 0.576787 |
| Dhh | 4.425596 | 3.392188 | Wnt1 | 1.307538 | 1.156752 |
| Gjb1 | 1.920556 | 0.789268 | Dll1 | 1.148384 | 0.601116 |
| Ncam1 | 6.068963 | 0.662156 | Nes | 0.219374 | 0.013968 |
| Neurog2 | 2.124203 | 1.844036 | Prom1 | 0.663089 | 0.093934 |
| Notch1 | 1.539392 | 0.374914 | Stat3 | 1.771547 | 0.008683 |

¹The data in the AVG columns present fold changes of expression in MEFs as compared to TKOs (individual levels normalized based on ACTB expression levels.
n.d., not determined as the gene product was not detected in one or the other sample.

Induction of mRNAs for markers of all three embryonic layers was seen in the sphere-derived cells (see also FIGS. 7 and 16A-16C). These markers included important developmental transcription factors such as GATA4, T, Msx1, Foxa2, MyoD, Ascl2, PDX1, PPAR and islet1, and components of development signaling pathways including TGF−/BMP, notch, wnt, and FGF (FIGS. 7 and 16A-16F). They also included markers of terminal differentiation such as cardiac actin, myosin heavy chain, osteocalcin, aggrecan, E-cadherin, transferrin, α-fetoprotein (AFP), myelin basic protein, GFAP, tyrosine hydroxylase, β-III tubulin, NCAM, Neurog2, Col9a1, CD19, CD3, CD4, and CD8.

Next, spheres were fixed and sectioned for immunostaining. The perimeter of embryoid bodies formed from ES cells typically contain early endodermal cells characterized by expression of AFP and GATA4, and this region is a site of hematopoietic and endothelial differentiation resembling embryonic yolk sac blood islands (Burkert et al., 1991). A band of cells was observed around the perimeter of RB1^{-/-} MEF spheres which resembled endodermal cells (FIGS. 17A-17C), and these cells immunostained for AFP (FIGS. 17D and 17E). This region also immunostained positively for GATA4 protein, and mRNAs for GATA4 and the early endodermal transcription factors Foxa2, PDX1, and Isl1 were also induced in spheres (FIGS. 7, 17A, and 18).

This region of the spheres also contained a number of cells with eosinophilic cytoplasm, and these cells immunostained for globin, indicating that they were erythroid (see FIGS. 17F-17H and 19). While most of these globin⁺ cells were nucleated, some of the cells lacked nuclei (FIGS. 17H and 19), implying that they might have been progressing from erythroblast like progenitors toward erythrocytes in the spheres.

This perimeter region of the spheres also contained cells with elongated morphology resembling endothelial cells (FIGS. 17A-17C), and indeed these cells immunostained for the endothelial marker CD31 (FIGS. 17I and 17J).

Although less abundant than the globin⁺ cells, cells with morphologies of other hematopoietic lineages, including megakaryocytes, were also evident (see FIGS. 19A-19S). Flow cytometry of total sphere-derived cells revealed that approximately 2% of the population expressed the hematopoietic stem cell marker CD34 and approximately 1% expressed the B cell marker CD19. CD34 and CD19 mRNAs were also induced in the spheres (FIG. 16C). Taken together, these results provided evidence that, as in embryoid bodies, the perimeter of the spheres was a site of hematopoietic/endothelial differentiation.

As erythrocytes mature they lose their nuclei. FIGS. 19A-19L show that the cells in spheres differentiated to form erythrocytes at various stages of differentiation, some of which have nuclei and some of which have lost their nuclei. FIGS. 19M-19Q show immunostaining for hemoglobin demonstrating that the forming erythrocytes expressed hemoglobin. Other cells of hematopoietic origin were also evident in the spheres. FIGS. 19R and 19S show a megakaryocyte. Together, these results demonstrated that cells in the spheres differentiated into various hematopoietic lineages, which is also a characteristic of ES cells and iPSC cells.

Cells interior to the globin⁺ cells in spheres displayed epithelial-like morphology (FIGS. 17A and 17C), and these cells expressed the early epithelial marker E-cadherin (cdh1; see FIG. 17K). In addition to upregulation of cdh1, expression of the epithelial progenitor marker Ker15 was also induced (FIG. 7). Immunostaining for the neuronal marker β-III tubulin was also observed (FIG. 17L). These β-III tubulin⁺ cells were generally in clusters or spherical structures. Immunostaining for all of the markers of differentiation increased in a time dependent fashion from 4 days in suspension culture out to at least 24 days. By 24 days, a higher percentage of the β-III tubulin⁺ cells exhibited elongated morphology characteristic of neurons.

Similar staining for globin, AFP, CD31 was also seen in the periphery of spheres derived from TKO cells. Again, β-III tubulin⁺ cells were found primarily in clusters containing cells with neuronal morphology, and cells in these clusters also expressed α-tyrosine hydroxylase (a marker of dopaminergic neurons; FIG. 18). Cells surrounding some of these neuronal clusters showed elongated projections and immunostained for both tyrosine hydroxylase and the motor neuron marker isl1 (FIG. 18). In addition to these neuronal markers, immunostaining for markers of oligodendrocytes (myelin basic protein) and glia/astrocytes (GFAP) was also evident in distinct regions of the spheres (FIG. 18). Expression of these neural markers was consistent with the induction of mRNA for various neural markers in the spheres (FIGS. 7 and 16B).

Based on these Real Time PCR and immunostaining results, it appeared that in addition to generation of cells with SP properties, sphere formation in RB1^{-/-} and TKO MEF spheres triggered differentiation into cells representative of all three embryonic layers.

### Example 8

### SP Cells Form Tumors in Nude Mice

Because sphere formation in TKO and RB1^{-/-} MEFs led to cells with properties of cancer stem cells in culture, whether these cells could form tumors in vivo was tested. As a control, 100,000 trypsinized TKO cells from subconfluent monolayer culture were injected subcutaneously (s.c.) into the hind limbs of nude mice. Both early (passage 4) and late (passage 40) passage TKOs were employed. The results are summarized in Table 6.

TABLE 6
Tumor Formation In vivo by Injected Cells

| Cell | Number of Injected Cells | | | | | | | |
|---|---|---|---|---|---|---|---|---|
| Type | 100,000 | 50,000 | 20,000 | 5,000 | 2,000 | 1,000 | 500 | 100 |
| TKO | − | n.d. | n.d. | n.d. | n.d. | n.d. | n.d. | n.d. |
| TKO- | + | n.d. | n.d. | n.d. | n.d. | n.d. | n.d. | n.d. |
| SDC | | | | | | | | |
| MP | + | + | − | − | − | − | − | − |
| SP | n.d. | + | n.d. | + | + | + | + | + |
| TKO- | + | + | n.d. | n.d. | n.d. | n.d. | n.d. | n.d. |
| Ras | | | | | | | | |

n.d.: not determined;
TKO-SDC: TKO sphere-derived cells containing approximately 10% SP and 90% MP cells (see FIGURE 8C).

Tumors did not form in the mice, even after two months, when TKOs from a subconfluent monolayer culture that had not gone through sphere formation were injected s.c. into the hind limbs of nude mice. Nor did these cells or RB1^{-/-} MEFs form colonies in soft agar (FIG. 3). However, injection of small spheres of TKOs or RB1^{-/-} MEFs after two weeks in suspension culture led to tumor formation. Examples of tumor formation in nude mice are shown in FIGS. 20A and 20B.

50,000 sphere-derived TKOs or RB1^{-/-} MEFs, which had migrated from spheres to reform monolayers, were also injected. These cells were trypsinized from culture plates and compared to an equal number of TKO-Ras cells for the ability to form tumors. Tumors were harvested after 31 days. TKO-Ras cells formed tumors (average tumor mass=515±104 mg), and the different tumors were histologically indistinguishable and they appeared to be spindle cell sarcomas (FIG. 20C). The sphere-derived TKO and RB1^{-/-} MEF cells also formed tumors (500±18 mg). Histologically, the tumors formed from small spheres or sphere-derived cells were indistinguishable, and tumors from TKO or RB1^{-/-} sphere-derived cells were also indistinguishable (compare FIGS. 21A-21D). These tumors also appeared to be spindle cell sarcomas similar to those formed with TKO-Ras cell.

However, tumors from sphere-derived cells also contained sphere-like whorls with eosinophilic centers (which were not evident in TKO-Ras tumors; FIGS. 20C and 21). These sphere-like whorls appeared histologically similar to regions evident in spheres in culture that expressed neuronal markers (FIG. 18). Indeed, immunostaining of tumor sections revealed that these whorls expressed -III tubulin, and as with spheres in culture, no other regions of the tumor expressed -III tubulin (FIG. 21). No -III tubulin expression was seen in TKO-Ras tumors. Tumors resulting from injection of sphere-derived cells from TKO or RB1^{-/-} MEFs also showed clusters of cells with nuclear immunostaining for Oct4 and Nanog, suggesting that the Oct4- and Nanog-expressing SP cells were retained in these tumors.

SP cells were originally identified as the subpopulation of tumor cells capable of efficiently regenerating the tumor when transplanted into second recipients. Therefore, different numbers of sorted SP and MP cells were injected into nude mice to assess which population was tumorigenic. Two independent experiments were performed with two injections of each cell number in the following experiments. Initially, 50,000, 20,000, 5,000, or 1,000 MP cells were injected. While tumors formed with each injection of 50,000 MP cells (523±93 mg after 31 days), no tumors were observed in any injection with 20,000 or fewer MP cells, even after two months. However, when 5,000; 2,000; 500; or 100 SP cells were injected, tumors formed at each injection level and grew rapidly (e.g., 813±279 mg at three weeks with 100 SP cells injected).

Based on these results, it was concluded that SP cells were the primary initiators of tumor formation among the sphere derived cells. Even though the sorted MP population was initially devoid of SP cells, it is of note that a small percentage of SP cells (˜1%) became evident with passage of the MP population in culture, and this number of SP cells remained relatively constant for at least one month in culture (FIG. 11). Therefore, the appearance of a small percentage of SP cells among the MP population might account for tumor formation seen when the highest number of MP cells (50,000) was injected.

However, the tumors formed from SP and MP cells were histologically distinct (see FIGS. 20D-20F). The MP tumors were indistinguishable histologically from those formed with TKO-Ras cells (FIGS. 20C and 20D), whereas SP tumors contained neuronal whorls (FIGS. 20E and 20F). These whorls were similar in appearance to those seen in tumors derived from unsorted sphere-derived TKO or RB1^{-/-} cells (FIG. 21), but they were more numerous. They also immunostained for the neuronal marker β-III tubulin (FIGS. 20G and 20H). The SP tumors also contained clusters of cells expressing nuclear Oct4 and Nanog throughout the tumor (FIGS. 20I-20L), suggesting that SP cells were maintained in the forming tumor.

### Example 9

### Generation of Cells with Stem Cell Properties from Wild Type MEFs

The studies described herein above demonstrated that sphere formation could trigger reprogramming of fibroblasts with an RB1 pathway mutation to a phenotype resembling ES cells. However, these cells, in addition to producing differentiated cells, also produced cancer cells. Therefore, the same sphere formation procedure was performed with wild type MEFs and with human fibroblasts to determine whether sphere formation would produce the same reprogramming in these cells, but without the production of cancer cells that occurred with cells containing the RB1 pathway mutation.

Initially, wild type MEFs from E13.5 mouse embryos were isolated using standard techniques (see e.g., Nagy et al., 2003) and employed to form spheres. MEFs were grown to confluency, scraped from tissue culture plates, and placed in suspension as described herein above. Cells immediately formed spheres (see FIG. 22A) and these spheres were viable in culture for at least two months. RNA was isolated from the spheres and used in Real Time PCR assays. As described herein above, there was induction of mRNAs for several stem cell genes (see FIG. 22B).

Histological sections of spheres after one month in culture showed the presence of both nucleated and enucleated red blood cells that immunostained positively for globin and reacted with benzidine, which demonstrated the presence of hemoglobin in the cells. Megakaryocytes and neutrophils were also evident. Other bone marrow cells were also present. Immunostaining for β-III tubulin demonstrated the presence of neurons, and immunostaining for E-cadherin and ZO1 was evident on the surface of epithelial cells arranged in secretory ducts.

Immunostaining of MEF spheres is shown in FIG. 22C. Real Time PCR was also employed to assay expression of various markers associated with different cell types, and the results are presented in FIG. 22D.

Additionally, Hoechst⁻/Abcg2⁺/CD133⁺ SP cells have been isolated from wild type MEF spheres, and it was determined that the Hoechst⁻/Abcg2⁺/CD133⁺ SP cells were the cells that expressed stem cell markers. Additionally, these cells had an additional property that distinguished them from other cells in the spheres; they were small in diameter, ranging from 5-7 microns. Taken together, these results demonstrated that cells with a size and expression pattern substantially similar to that of stem cells could be generated from wild type MEFs after one week of culture as spheres in suspension culture.

When cultured under similar sphere-forming conditions, ES cells typically undergo differentiation into cells representative of all three embryonic layers. Indeed, the results disclosed herein demonstrated that mRNAs indicative of each of the three embryonic layers were induced in the spheres. Thus, stem cell-like cells in the spheres had the same property as ES cells in that they were capable of generating differentiated cells representing each of the three embryonic layers in spheres.

Similar studies were performed with human fibroblasts (see FIG. 23). These included primary cultures of human foreskin fibroblasts and primary cultures of fibroblasts from lung (e.g., cell lines IMR-90 and WI-38, both of which are available from the American Type Culture Collection (ATCC®), Manassas, Va., United States of America). FIG. 23A shows the presence of endodermal-like cells at the border of the sphere after H&E staining as evidenced by immunostaining with the endodermal marker a-fetoprotein (AFP; see FIG. 23E). These same cells were positive for the endothelial marker CD31 (see FIG. 23F) and α-globin (see FIG. 23G). Cells resembling nucleated blood cells were also present (see FIGS. 23B and 23C), which was confirmed by benzidine staining, which demonstrated the presence of hemoglobin (see FIG. 23D).

Furthermore, H&E stained sections (FIGS. 23H and 23I) showed the presence of endothelial cells (white arrow in FIG. 23I) surrounding a blood vessel, as well as a ductal structure (black arrow in FIG. 23I.

FIG. 23J shows benzidine staining of wild type MEF spheres. Benzidine staining demonstrated the presence of hemoglobin in cells of MEF spheres. FIG. 23K1 shows H&E staining of an erythrocyte, and FIG. 23K2 shows positive immunostaining of an adjacent section of the sphere for hemoglobin, demonstrating that this erythrocyte expressed hemoglobin. FIGS. 23L1-23L3 show positive immunostaining of another erythrocyte for hemoglobin, and this cell was nucleated as demonstrated by DAPI nuclear staining Thus, wild type MEF spheres contained both nucleated (i.e., immature) and enucleated (i.e., mature) erythrocytes.

FIGS. 23M1-23M3 show immunostaining for CD31, which is a marker of endothelial cells. DAPI staining was used to show the nuclei of the cells. CD31 staining demonstrated that endothelial cells were formed in the wild type MEF spheres, which also is known to occur in ES cell- and iPSC-derived spheres.

FIGS. 23N and 23O are photomicrographs showing a region of a wild type MEF-derived sphere containing cartilage, which is shown stained with alcian blue in FIG. 23O. FIG. 23P is a photomicrograph showing pearls of keratin (dark staining) in an keratinized cyst present within a wild type MEF-derived sphere.

Additionally, FIG. 24A is a photomicrograph showing a secretory epithelium ascinar-like structure with a central duct (arrow), and FIG. 24B shows evidence of the formation of secretory ducts (gray arrows) and red blood cells (white arrow). The top middle and top right photomicrographs of FIG. 24 show hair fibers at the border of the spheres (the border is identified by black arrows), and FIGS. 24C and 24D shows immunostaining for the epithelial marker E cadherin (Cdh1) and the neuronal marker β-III tubulin (β3Tub). FIGS. 24E and 24F (the latter an enlargement of the field in the box in FIG. 24E) show hair fibers at the border of the spheres (the border is identified by black arrows). These results demonstrated that wild type MEFs in spheres could differentiate into elaborate tissues and structures including hair and secretory epithelial structures, both of which are also properties of ES cells and iPSC.

And finally, FIGS. 25A-25Q are a series of photomicrographs of spheres produced by Hoechst⁻/Abcg2⁺/CD133⁺ cells derived from wild type MEFs after 2 weeks in culture. The Hoechst⁻/Abcg2^{|}/CD133^{|} cells were isolated by cell sorting and cultured on a feeder layer of irradiated fibroblasts. The wild type MEFs were isolated from β-actin-GFP transgenic mice obtained from The Jackson Laboratory (Bar Harbor, Me., United States of America). Cells in the center of the colonies maintained a Hoechst⁻ phenotype (characteristic of ES cells), whereas cells on the edges of the colonies became Hoechst⁺ (which is characteristic of differentiating cells). These Hoechst⁺ cells gave rise to a variety of differentiated cells that migrated away from the original colony. These differentiated cells expressed β-III tubulin (β3Tub), GFAP, Troponin I, CD34, CD45, AFP, ZO1, Ter119, or globin as shown in FIGS. 25D-25Q.

These results demonstrated that Hoechst⁻/Abcg2⁺/CD133⁺ cells derived from the wild type MEF spheres could be maintained in an undifferentiated state in culture, and that these cells could give rise to lineages representative of all three embryonic layers. These results also demonstrated that Hoechst⁻/Abcg2⁺/CD133⁺ cells expressed genes indicative of a variety of different lineages in monolayer culture: β-III tubulin indicative of neurons; GFAP indicative of glial cells; AFP indicative of endodermal cells; ZO1 indicative of epithelial cells; troponin I indicative of cardiomyocytes; CD34 and CD45 indicative of hematopoietic lineages; Ter119 indicative of erythrocyte progenitors; and globin indicative of erythrocytes. The ability of Hoechst⁻/Abcg2⁺/CD133⁺ cells from wild type MEF spheres to differentiate into a variety of lineages is shared by ES cells and iPSC. Thus, the cells behaved like ES cells and iPSC in monolayer culture as well as in spheres.

As such, sphere formation with both mouse and human fibroblasts led to expression of proteins indicative of all three embryonic layers. Further, the morphologies of the cells in these spheres were consistent with such differentiation. These results demonstrated that at the protein and morphology levels, mouse and human fibroblasts behaved like ES cells or induced pluripotent stem cells (iPSC) when induced to form spheres in that they gave rise to cells representative of all three embryonic layers.

### Example 10

### Teratoma Formation by Spheres and Sphere-Derived Cells

Small spheres and sphere-derived cells from wild type MEFs and human fibroblasts were injected into nude mice to assess tumor formation.

Four independent preparations of 50,000 cells were injected into both hind limbs of nude mice. The results are shown in FIGS. 26A-26E, which are a series of photomicrographs of teratoma formation by Hoechst⁻/Abcg2/CD133^{|} cells derived from wild type MEF spheres after 2 weeks in suspension culture. Tumors were observed in all 8 injections, and were tumors were collected after three weeks.

FIG. 26A is a Nomarski image of a representative teratoma, and FIG. 26B is a higher power view of an adjacent section of the tumor stained with H&E. A variety of structures characteristic of a teratoma can be seen. The MEFs were isolated from Actin-GFP mice and immunostaining for GFP (see FIG. 26D), which showed that the tumor was GFP⁺ whereas surrounding host tissue was GFP⁻. These results demonstrate Hoechst⁻/Abcg2⁺/CD133⁺ cells derived from wild type MEF spheres had another property of ES cells and iPSC: they formed teratomas.

Turning now to FIGS. 27A-27H, these Figures are a series of photomicrographs of teratomas formed with Hoechst⁻/Abcg2⁺/CD133⁺ cells derived from wild type MEF spheres showing cobblestone epithelial morphology and expressing the epithelial specification protein E-cadherin (see FIGS. 27C and 27D (low power) and 27G and 27H (higher power), which present E-cadherin immunostaining on the surface of the cells). These teratomas contained cells representative of all three embryonic layers as well as differentiated tissues, similar to teratoma formation by ES cells. Thus, Hoechst⁻/Abcg2⁻/CD133⁺ isolated from MEF-derived spheres formed teratomas containing differentiated epithelial cells.

Turning now to FIG. 28, FIG. 28A is a Nomarski image of adipose tissue present in a teratoma. 28B shows DAPI staining showing cell nuclei. FIG. 28C shows immunostaining for GFP showing that the adipose tissue was derived from the injected Hoechst⁻/Abcg2⁺/CD133⁺ cells. FIG. 28D is a merge of FIGS. 28B and 28C.

FIG. 28E is a Nomarski image of a neuronal structure in a teratoma. FIG. 28F shows DAPI nuclear staining of the section in FIG. 28D. FIG. 28G shows immunostaining of the section of FIG. 28E for β-III tubulin, showing a cluster of neurons within a neuronal structure in the teratoma. FIG. 28H is a merge of FIGS. 28F and 28G.

FIG. 28I is a Nomarski image of a region of intestinal-like epithelium in a teratoma. FIG. 28J shows DAPI nuclear staining of the section of FIG. 28I. FIG. 28K shows immunostaining of the cells presented in FIG. 28I for GFP, and shows that this intestinal-like structure was derived from injected Hoechst⁻/Abcg2⁺/CD133⁺ cells. FIG. 28L is a merge of FIGS. 28J and 28K.

FIG. 28M is a Nomarski image of a secretory epithelial structure in a teratoma. FIG. 28N shows DAPI nuclear staining in the structure of FIG. 28M. FIG. 28O shows GFP immunostaining and demonstrated that the structure in FIG. 28M is derived from the injected Hoechst⁻/Abcg2⁺/CD133⁺ cells. FIG. 28P shows the results of immunostaining the structure for CDH1 expression, which demonstrated that the structure was epithelial.

FIGS. 29A-29I are a series of photomicrographs showing formation of skeletal muscle in a teratoma derived from Hoechst⁻/Abcg2⁻/CD133⁺ cells derived from wild type MEF spheres injected into nude mice. FIG. 29A shows skeletal muscle fibers in the teratoma by H&E staining. A Nomarski image of an adjacent section is shown as FIG. 29B and GFP staining is shown in FIG. 29D, demonstrating that the muscle cells ware tumor-derived.

Control photomicrographs are presented in FIGS. 29F-29I. A Nomarski image of host skeletal muscle is shown in FIG. 29F. DAPI staining is shown in FIG. 29G and GFP is shown in FIG. 29H. There was a lack of GFP staining in FIG. 29H, which shows host muscle that does not express GFP, indicating that Hoechst⁻/Abcg2⁺/CD133⁺ cells derived from wild type MEF spheres formed teratomas in nude mice containing skeletal muscle, which is also known to occur with teratomas derived from ES cells.

Thus, the experiments disclosed herein demonstrated the presence of multiple differentiated tissues in teratomas formed with Hoechst⁻/Abcg2⁺/CD133⁺ cells derived from wild type MEF cells following sphere formation. These results further demonstrated that the Hoechst⁻/Abcg2⁺/CD133⁺ cells derived from wild type MEF spheres had properties of ES cells and iPSC. Thus, sphere formation was able to generate reprogrammed fibroblasts that does not rely on re-expression of exogenous stem cells genes. Instead, this technique led to re-induction of endogenous stem cell genes to reprogram the wild type MEFs.

Summarily, none of the wild type cells produced tumors. This sphere-dependent reprogramming of the wild type fibroblasts thus did not appear to produce cancer cells as was observed in cells in which the RB1 pathway was mutated.

### Example 11

### Production of Melanocyte-Like Cells from MEF Spheres

MEF spheres were transferred to tissue culture dishes after two weeks in suspension culture. Spheres attached to the plates and cells began to migrate out of the spheres and onto the plate as was observed with TKO and RB1^{-/-} MEF spheres. However, in contrast to the TKO and RB1^{-/-} MEF cells, only a portion of the cells from the wild type MEF spheres migrated back onto the plate. These cells were highly pigmented (see FIGS. 30A-30C). Initially, most of the cells were rounded or epithelial in appearance. However, after several days on the plates, the cells remained pigmented and began to elongate (see FIGS. 30D-30H). After several more days, the cells were still pigmented but then began to send out multiple dendritic-like projections resembling melanocytes.

The cells were immunostained for two melanocyte-specific markers: Mitf and mel5, and the results are presented in FIGS. 30I-30K. All of the pigmented cells immunostained positively for both markers, indicating that the pigmented cells which migrated out of the MEF spheres were melanosome-like and that they took on the morphology and gene expression pattern of melanocytes after several days in culture.

Similar results were seen with spheres formed from human foreskin fibroblasts and with the normal human lung fibroblast lines IMR-90 and WI-38 obtained from the American Type Culture Collection (ATCC®; Manassas, Va., United States of America).

### Example 12

### Gene Expression Analysis of Melanocyte-Like Cells from MEF Spheres

RNA was isolated from melanocyte-like cells from MEF spheres and used for Real Time PCR comparison to MEF maintained as subconfluent monolayers using the primers disclosed in Table 4. Tyr and Tyrp1 are key genes in the pigment synthesis cascade. Pax3 and Sox10 cooperate with the MITF-M isoform in the specification of melanocytes. RPE65 is a marker of retinal pigment epithelial cells, which is not expressed in melanocytes and thus was employed as a control. Taken together, the results shown in FIGS. 30A-30F and 31 demonstrated the efficient formation of melanocytes from mouse and human fibroblasts via sphere formation since the cells were Tyr⁺, Tyrp1⁺, Pax3⁺, Sox10⁺, and Mitf M⁺, while being RPE65 negative.

MEFs, human foreskin fibroblasts, and the normal human lung fibroblast cell lines IMR-90 and WI-38 were individually grown to confluence and then scraped from tissue culture plates and placed in suspension culture in non-adherent plates. After two weeks in culture, the resulting spheres were transferred to culture dishes. As with TKO and RB1 null MEFs, cells in the spheres migrated back onto the tissue culture dishes to reform monolayers. However, in contrast to the mutant MEFs, not all of the cells in the wild type spheres migrated back out of the spheres.

The cells migrating out of the spheres were highly pigmented, and results shown in FIGS. 30A-30F and 31 suggested that these pigment cells were melanocyte precursors which subsequently sent out dendritic process and differentiated into melanocytes following re-adhesion to the tissue culture dish. This conclusion is based both on morphology (dentritic processes and pigment) and expression of the melanocyte-specific markers Mift-M and Mel5 (see FIGS. 30I-30K) and the melanocyte specification genes Sox10 and Pax3.

Because highly pigmented melanocyte precursors are the primary cell type that migrated from the wild type mouse and human spheres, these cells could be obtained in relatively pure form.

Antibody information: Mitf and mel5 (tyrosinase related protein 75) antibodies were from Abcam Inc., Cambridge, Mass., United States of America and were used at a dilution of 1:50 as described by the manufacturer.

### Example 13

### Sphere Formation using Human Lung Bronchial Epithelial Cells

Primary cultures of human lung bronchial epithelial cells were grown to confluence, and then scraped from tissue culture dishes and placed in suspension culture in non-adherent plates as described herein above for fibroblasts. Spheres were allowed to form for 5 days, and then the spheres were fixed and sectioned into 5 micron sections. The results of analyses of these spheres are presented in FIGS. 32A-32J, which present a series of photomicrographs showing primary cultures of human lung bronchial epithelial cells grown to confluence, scraped from tissue culture dishes, and placed in suspension culture in non-adherent plates as described herein for fibroblasts.

FIGS. 32A-32C show sections of an exemplary human lung bronchial epithelial cell-derived sphere stained with H&E (FIG. 32A), immunostained for the presence of globin (FIG. 32B), and a merge of the H&E and immunostained fields (FIG. 32C). Erythrocyte differentiation was identified in the spheres. FIGS. 32D-32I show higher power views of an exemplary human lung bronchial epithelial cell-derived sphere showing erythrocytes immunostaining positively for hemoglobin.

Spheres were also stained with benzidine to test for the presence of hemoglobin. FIG. 32J shows an exemplary benzidine staining of a section of an exemplary sphere, which showed the presence of hemoglobin. These results demonstrated that wild type human lung epithelial cells can also form spheres in suspension and undergo differentiation into erythrocytes expressing hemoglobin. These spheres also showed cells with a variety of morphologies, suggesting that like wild type MEFs and human foreskin fibroblasts, the epithelial cells could also undergo differentiation into a variety of cells types in the spheres, thereby extending the presently disclosed sphere formation technique to wild type human epithelial cells.

Summarily, the spheres appeared morphologically similar to those formed from fibroblasts, and the efficiency of sphere formation in the epithelial cells and fibroblasts was similar. Also as with the fibroblast spheres, the human lung bronchial epithelial cell-derived spheres contained a number of nucleated and non-nucleated eosinophilic cells resembling erythrocytes and erythrocyte progenitors similar to those seen with spheres generated from fibroblasts. Sections of the human lung bronchial epithelial cell-derived spheres immunostained positively for the a-globin chain of hemoglobin, and the benzidine-peroxide stain produced a dark blue reaction in the presence of hemoglobin (see arrows in FIG. 32J).

As such, human lung epithelial cells could also form spheres in suspension culture and underwent a similar differentiation into cells resembling erythrocytes as seen with fibroblast spheres. As such, it appeared that epithelial cells induced to form spheres in suspension also underwent reprogramming and differentiated into other cell types.

### Example 14

### Expansion of Sphere-Induced Pluripotent Stem-Like (siPS) Cells

Wild type primary mouse embryonic fibroblasts (MEFs), mouse adult skin fibroblasts (MAFs), and mouse tail-tip fibroblasts (TTFs; passage>7 in all cases) were obtained from pure inbred C57BL/6 mice as described previously (Liu et al., 2008, the disclosure of which is incorporated herein by reference in its entirety). MEFs were obtained from E15.5-E17.5 embryos of two different lines—one that expressed an enhanced green fluorescent protein (EGFP) transgene and a second that lacked the EGFP transgene. MAFs were obtained from David Johnson (University of Texas M.D. Anderson Cancer Center, Houston, Tex.). TTFs were obtained from 4-day old mouse tail tips of the same strain as the MEFs with the EGFP transgene. All mice were from a C57BL/6 genetic background. Primary murine fibroblasts (MEFs, MAFs, and TTFs) were cultured in standard DMEM medium with 10% GIBCO® fetal bovine serum (FBS; available from INVITROGEN™ Corp., Carlsbad, Calif., United States of America). Medium was refreshed as needed.

Murine ES (W95) and siPS cells were cultured on STO-Neo-LIF (SNL) feeder cells in complete ES cell medium, which was DMEM (high glucose) supplemented with 15% FBS, LIF (1,000 units/ml), 2 mM non-essential amino acids, 2 mM GIBCO® GLUTAMAX™ (INVITROGEN™ Corp), 0.1 mM β-mercaptoethanol, and 1× nucleosides (100× nucleosides stock is 40 mg adenosine, 42.5 mg guanosine, 36.5 mg cytidine, 36.5 mg uridine, and 12 mg thymidine dissolved in 50 ml double distilled water). W95 ES cells were derived from C57BL/6 blastocysts. Medium was refreshed every other day.

Reprogramming of primary MEFs was performed as described herein with the following modifications. Briefly, 10-cm tissue culture plates were coated with 0.1% gelatin for 1 hour at 37° C. SNL feeder cells that had been irradiated with 4,500 rads of gamma irradiation were seeded onto 12-well tissue culture plates and cultured in DMEM medium with 10% FBS overnight. Primary cells prepared as described herein above were cultured in DMEM medium with 10% FBS, and were split 1:1 when they became confluent. On the day after splitting, fast-growing cells were scraped off the plate with a scraper, spun down at 300 g for 5 minutes, and re-suspended in 1 ml of complete mouse ES cell medium. The cells were individualized thoroughly by pipetting up down a few times with a PIPETMAN® P-1000 pipette (Rainin Instrument, LLC, Oakland, Calif., United States of America) and transferred to a 3-cm non-adherent plate with 2-3 ml of complete mouse ES cell medium to form spheres.

Well-isolated spheres at 2 to 7 days in suspension were transferred to the 12-well SNL feeder plate containing complete mouse ES cell medium. 2-10 spheres were seeded into each well for generation of siPS. Cultures were maintained in mouse ES cell medium, which was changed every other day. From day 6 to day 15 after the spheres were transferred, colonies with ES cell-like morphologies became visible and were scored. Colonies were picked when they had increased to a sufficient size and were expanded on feeder fibroblasts using standard procedures.

### Example 15

### Reprogramming of siPS Cells

For quantification of siPS cell generation efficiency, a 10-cm plate of monolayer fibroblast cells of approximately 1×10⁶ in total that could form about 200 spheres in a 3-ml suspension culture was employed. Out of a total of about 400 colonies formed, approximately 20 very good quality ES-like colonies were typically generated. These colonies were further expanded into and maintained as cell lines. Compared to the mouse ES cell line W95, these sphere-formed colony cells were confirmed to be siPS by immunostaining, RT-PCR, in vitro directed differentiation into various types of differentiated cells, in vivo teratoma formation in nude mice, genome expression profiling, and chimeric mouse production as follows. Particularly, immunostaining for ES specification factors (e.g., Oct4, and Nanog) was similar, and the levels of mRNAs for the stem cell factor genes were similar between the siPS and W95 ES cells. Additionally, both cell populations formed teratomas in nude mice, the microarray array gene expression profiles were similar (they profiles were also similar to published ES and iPSCmicroarray gene expression profiles), and like ES and iPSC, siPS generated chimeric mice when introduced into mouse embryos.

Immunofluorescence. siPS cells were grown on SNL feeder cells in chamber slides coated with 0.1% gelatin in complete mouse ES medium as described herein above. At days 3 when colonies started to appear, cells were fixed with 3.7% paraformaldehyde for 30 minutes at room temperature, washed once with 1× PBS buffer, and permeabilized with PBS containing 0.02% Tween-20 for 30 minutes. Cells were blocked in PBS with 4% serum as set forth in Liu et al., 2009 (incorporated herein by reference in its entirety) plus 2% bovine serum albumin (BSA) for 1 hour at room temperature (RT) and then incubated with antibodies against Oct3/4, Nanog, and Ssea1 overnight at 4° C. The next day, cells were washed in PBS and incubated with Alexa Fluor 488-conjugated anti-mouse secondary antibodies (1:500). Cells were also stained with a nuclear-staining Hoechst dye (1:500). Images were recorded under a Zeiss fluorescence microscope.

Whole mouse gene expression profiling. Whole genome expression profiling patterns of siPS cells were compared to those of the original cell lines from which they were derived and also to those of a wild type embryonic stem cell line (W95) using an Agilent whole mouse gene expression microarray (4×44K genes, 60-mer arrays, Agilent Technologies, Santa Clara, United States of America). A heat-map of the gene expression profiling results was constructed to compare gene expression patterns in the siPS an in the W95 ES cell line.

Quantitative Real Time PCR. Total RNA from cells was extracted with Trizol (Invitrogen Corp., Carlsbad, Calif., United States of America). Samples were treated with DNase I before reverse transcription using random primers and Superscript Reverse Transcriptase (INVITROGEN™ Corp.), according to the manufacturer's protocols. Quantitative Real Time PCR was performed using a Stratagene Mx3000P qPCR System (Agilent) an a DNA Master SYBR Green I mix (Bio-Rad Laboratories, Hercules, Calif., United States of America). All values were obtained in at least three replicates and in a total of at least two independent assays.

Differentiation of siPS cells into photo receptor neural cells with MATRIGEL™ in vitro. Differentiation of cells in MATRIGEL™ was performed. Cultures were grown to near confluency in complete mouse ES medium with LIF (day 0), and then trypsinized and seeded at a lower density in the absence of LIF for 1 day (day 1). The cells were cultured and passaged on an irradiated mouse embryonic fibroblast feeder layer.

Retinal induction was also performed. Briefly, embryoid bodies (EBs) were formed by scraping siPS from plates, pipetting with a PIPETMAN® P-200 pipette (Rainin Instrument, LLC, Oakland, Calif., United States of America) to disrupt the colonies and resuspending the cells at a concentration of approximately 100,000 cell per ml in a 6 well ultra-low attachment plate (VWR international, Radnor, Pa., United States of America). EBs were cultured for 3 days in the presence of mouse noggin (R&D Systems, Minneapolis, Minn., United States of America), human recombinant Dkk-1 (R&D Systems), and human recombinant insulin-like growth factor-1 (IGF-1; R&D Systems). On the fourth day, embryoid bodies were plated onto poly-D-lysine-MATRIGEL™ (Becton Dickinson, Franklin Lakes, N.J., United States of America) coated plates and cultured in the presence of DMEM/F 12, B-27 supplement, N-2 Supplement (INVITROGEN™ Corp.), mouse noggin, human recombinant Dkk-1, human recombinant IGF-1, and human recombinant basic fibroblast growth factor (bFGF; R&D Systems). In particular, the media contained DMEM/F12, 10% knockout serum replacer, N2 supplement, B27 supplement, 1 ng/ml DKK1 (R&D Systems), 1 ng/ml noggin (R&D Systems), and 1 ng/ml IGF-1 (R&D Systems), and the culturing was for three days. Then, embryoid bodies were transferred to poly-D-lysine coated plates with undiluted MATRIGEL™ and they were culture for 21 days in media containing 10 ng/ml DKK1, 10 ng/ml NOGGIN, 10 ng/ml IGF-1, and 5 ng/ml human recombinant bFGF (R&D Systems). The media was changed every 2-3 days for up to 3 weeks.

Teratoma formation. 1×10⁵ siPS cells were subcutaneously injected into irradiated (4 Gy) nude mice. Injections were performed 1 day after irradiation. Teratomas were surgically removed after 3 weeks. Tissue was fixed in formalin at 4° C., embedded in paraffin wax, and sectioned at a thickness of 5 μm. Sections were stained with hematoxylin and eosin (H&E) for pathological examination, or processed for immunohistochemical analysis with antibodies against EGFP or the following markers of differentiation: beta III tubulin for neuroectoderm, α-fetoprotein for mesoderm, and CD31 for endoderm.

Chimera formation. The ability of siPS cell clones to generate chimeras in vivo is tested by microinjection into C57BL/6J-Tyr^{C-2J}/J (albino) blastocysts, or by aggregation with CD1 (albino) morulae according to standard protocols (see e.g., Nagy et al., 2003. See also EXAMPLES 20 and 21, below.

### Example 16

### Generation of Sphere-Induced Pluripotent Cells (siPS)

FIG. 34 shows the results of generating siPS as set forth in EXAMPLE 15 using fibroblasts from the skin of neonatal mice placed in tissue culture. The cells were immunostained for the stem cell markers Oct4, Nanog, and Ssea1 (FIGS. 34A, 34C, and 34E, respectively). No immunostaining was detected, indicating that the skin fibroblasts did not contain any ES cell-like cells.

Spheres were formed from the fibroblasts as described in detail herein above. After 2 weeks in suspension culture the spheres were fixed, sectioned, and the sections were immunostained for the stem cell markers. Immunostaining demonstrated that sphere formation induced the generation of cells expressing stem cell markers. Higher power magnifications of FIGS. 34A, 34C, and 34E are shown in FIGS. 34B, 34D, and 34F, respectively. Blue DAPI nuclear staining was observed in FIG. 34B, panel 3; 34D panel 3 when these fields were viewed in color; and 34F, panel 3. Oct4 and Nanog are transcription factors that were located in the nucleus, whereas Ssea1 was found on the cell surface. Pair-wise comparisons of the staining in FIGS. 34A and 3B, 34C and 34D, and 34E and 34F show that sphere formation led to high level induction of the Oct4, Nanog, and Ssea1 markers of pluripotent cells.

Spheres were formed in culture for times ranging from 3 days to 7 days. Spheres were then allowed to attach to a plate of irradiated fibroblast feeder cells as shown in FIG. 34G. These plates were maintained in standard stem cell media which contains LIF for mouse cells and fibroblast growth factor for human foreskin fibroblasts. The sphere in FIG. 34G was 7 days old and derived from fibroblasts isolated from mouse tail skin. One day after attachment to the feeder layer, cells start to migrate out of the sphere (FIG. 34H). After two weeks, colonies resembling embryonic stem cells formed (FIG. 34I). Arrows in FIG. 34I denote stem cell colonies. These colonies could be passaged by treating with trypsin and transferring to new plates of feeder layer cells. FIG. 34J shows a colony that immunostained for Ki67, which is a marker of cell proliferation, thus demonstrating that the cells in the colonies were dividing. Colonies positively immunostained for Oct4 and Nanog (see FIGS. 34K and 34L, respectively), demonstrating that like embryonic stem cells, they expressed these stem cell factors.

### Example 17

### Gene Expression Profiling of siPS

FIG. 35 shows the results of global gene expression profiling of siPS exemplified by those shown in FIG. 34, which resembled that of embryonic stem cells. Microarray-based gene expression analysis using Affymetrix Gene Chips assessed gene expression in siPS, embryonic stem cells (W95), and the fibroblast cell lines (MEFs) from which the siPS were derived. FIG. 35 shows heat maps for 15,000 genes for which expression changed more than 1.5-fold compared to MEFs. This quantitative assessment demonstrated that the gene expression profiles of siPS closely resembled those of embryonic stem cells and that they were different from the parent MEFs.

### Example 18

### Tumor Formation of Transplanted siPS

50,000 siPS were injected into the hind limbs of nude mice as described herein above. After 3 weeks, tumors formed in both hind limbs of all three injected mice, and they were removed for histology. Frozen sections were stained with H&E, and a representative section is shown in FIG. 36.

As shown in FIG. 36, these tumors were teratomas. Tissues representative of all three embryonic layers were present in the tumor. It is noted that teratoma formation is generally considered an important criterion for induced pluripotent stem cell formation.

### Example 19

### Generation of Human siPS

Human foreskin fibroblasts were employed to generate human siPS essentially as described above with the following modification. After the sphere were formed and re-plated on irradiated fibroblasts, the medium in which the human siPS were generated was a human ES cell medium that contained FGF rather than LIF which was employed in mouse ES cell medium.

### Example 20

### Generation of Chimeric Mice with siPS

The capacity of the sphere-induced pluripotent cells (siPS) generated from mouse embryonic fibroblasts (MEFs) derived from male embryonic day 18.5 (E18.5) C57BL/6 to generate chimeras in vivo was tested by microinjection of siPS into C57BL/6J-TyrC-2J/J (albino) blastocysts. For each injection preparation, several siPS colonies were selected, trypsinized, and resuspended in the ES cell culture medium. Seven different siPS preparations and a total of about 150 blastocyst microinjections were performed, each with 6-10 siPS injected per blastocyst. Injected blastocysts were implanted into pseudopregnant albino females. The chimeric mice were identified initially by coat, whisker, and eye color, wherein the C57BL/6-derived siPS contributed black coloring against the albino background derived from the C57BL/6J-TyrC-2J/J blastocysts.

FIGS. 37A-37G are photographs of exemplary chimeras produced as described herein. As seen in FIGS. 37A-37G, the siPS contributed to coat color (see e.g., FIGS. 37A, 37B, 37E, and 37F) and eye color (see e.g., FIGS. 37A and 37E-37G), particularly with respect to the retinal pigmented epithelium (RPE; see FIGS. 37C and 37D) of chimeric mice. Y chromosome painting using a Cy3-labeled reagent that detects cells containing a Y chromosome demonstrated extensive contribution of siPS-derived cells to the eye of chimeric mice (see the pink staining of FIG. 37D).

To determine the contribution of the siPS cells to various tissues in the chimeras, pregnant mice following blastocyst injection were sacrificed at embryonic day 15 (E15) and anatomically female embryos were collected and sectioned for Y chromosome painting. Embryos were employed to facilitate sectioning through multiple tissues. Female embryos were analyzed so that the contribution of the male siPS could be assessed. The results are summarized in Table 7 below.

TABLE 7
Contributions of Male siPS to Somatic Tissues of Female Chimeric Mice

| Tissue | Percent Male Cells |
|---|---|
| Heart | 27 ± 4 |
| Brain | 82 ± 22 |
| Retina | 58 ± 5 |
| Intestine | 25 ± 7 |
| Vertebrae | 45 ± 16 |
| Spinal Cord | 78 ± 15 |
| Lung | 16 ± 9 |
| Liver | 19 ± 7 |
| Limb | 26 ± 13 |

### Example 21

### Generation of Germline Chimeric Mice with siPS

Employing the basic techniques discussed herein above in EXAMPLE 20, sphere-induced pluripotent cells (siPS) generated from mouse embryonic fibroblasts (MEFs) derived from male embryonic day 18.5 (E18.5) C57BL/6 are used to generate chimeras by microinjection of siPS into C57BL/6J-TyrC-2J/J (albino) blastocysts. Injected blastocysts are implanted into pseudopregnant albino females, and chimeric mice are allowed to develop to term and be born. Chimeric mice are identified by coat color analysis, and upon reaching sexual maturity, are test bred to albino mice. Pups born from the mating of a chimera and an albino mouse are observed after birth to identify those pups that have black coat color, which is indicative of the chimera that is its parent having gametes derived from the siPS and is indicative of the ability of the siPS to contribute to the murine germline.

### Discussion of the EXAMPLES

Embryonic stem (ES) cells and induced pluripotent stem cells (iPSC) can typically differentiate into cells representing each of the three embryonic lineages (ectoderm, endoderm, and mesoderm) when placed in suspension culture, and this differentiation is accompanied by activation of signaling pathways including Wnt, Notch, and growth factors such as BMP and FGF. The Real Time PCR results disclosed herein demonstrated that TKO cells placed in spheres can, like ES cells and iPSC, differentiate into cells expressing mRNAs for markers of all three embryonic layers. The results also demonstrated that TKO induced to form spheres expressed mRNA for genes associated with Wnt, Notch, and growth factor signaling that are known to drive these types of differentiation. In this way, TKO cells resembled ESC and iPSC.

However, TKO cells could also give rise to cancer cells, suggesting that mutation of the RB1 family might associated with cancer generation in these cells. It is also disclosed herein that wild type MEFs without the RB1 family mutations (i.e., that are RB1⁺, RBL1⁺, and RBL2⁺) also differentiated into cells expressing mRNAs for markers of all three embryonic layers, but did not give rise to cancer cells in the same fashion as did TKO MEFs.

When the RB1 pathway was mutated, these reprogrammed cells gave rise to both differentiated cells as well as cancer stem cells, which in turn gave rise to cancer cells. Additionally, sphere formation using wild type mouse or human fibroblasts led to similar reprogramming, but cancer cells were not produced. Thus, maintaining a functional RB1 pathway could prevent the production of cancer cells during reprogramming of fibroblast via sphere formation.

Sphere formation can provide reprogramming, but since the endogenous stem cell genes were re-expressed (i.e., without requiring ectopic expression from recombinant vectors), there was no need for viral infection and its associated cancer risk.

Undifferentiated ES cells form teratomas when injected into hosts, thus these cells must be partially differentiated in culture prior to injection. Nevertheless, a cancer risk remains from any remaining undifferentiated cells. Additionally, partial differentiation of ES cells seems to be required for their ability to facilitate repair of tissues in vivo. Sphere-derived cells from wild type mouse or human fibroblasts did not appear to pose a cancer risk. Therefore, progenitors representative of cells in all three embryonic layers can be sorted from spheres using specific cell surface markers and can be used in similar therapies as partially differentiated ES cells or induced pluripotent fibroblasts.

Based on the discoveries described herein, cells in spheres can be directed toward specific differentiation pathways by using the various differentiation protocols that have been established for ES cells. An exemplary approach is that skin fibroblasts from a patient following punch biopsy are placed in culture and used to form spheres. During or following sphere formation, the sphere derived cells can be exposed to appropriate growth factors and cytokines designed to enhance and/or facilitate formation of a specific cellular lineage. Cells surface markers specific for this lineage can be used to sort the differentiated cells, which can then in turn be used therapeutically in cell transfer back to the patient. These transfer experiments are analogous to those currently underway with ES cells and induced pluripotent fibroblasts.

Exemplary advantages of employing the presently disclosed cells rather than ES cells include, but are not limited to the fact that the former are not characterized by the ethical concerns raised by use of the latter, apparently have greatly reduced or no risk of teratoma formation, and would not give rise to histocompatibility issues (or other genetic or infection issues) because the sphere-derived cells can be isolated from the subject into which they would thereafter be introduced (unlike ES cells).

Another advantage that the induced pluripotent fibroblasts disclosed herein would be expected to have over ES cells is that endogenous “pluripotency markers” (e.g., Oct4, Sox2, and Klf4) are caused to be re-expressed in the sphere-derived cells without the need to resort to employing viral infection, which has been linked to cancer risk.

As disclosed herein, sphere formation is a mechanism for reprogramming of fibroblasts to a multipotential phenotype. While the instant co-inventors do not wish to be bound by any particular theory of operation, a proposed model for a pathway for generation of cells with properties of cancer stem cells from differentiated somatic cells is presented in FIG. 33.

Summarily, the experiments disclosed herein provided evidence that siPS could be generated from fibroblasts by forcing the cells to form spheres. Additionally, siPS can be isolated by plating the spheres that form onto feeder layers and allowing the siPS to migrate out of the sphere and form colonies. These colonies can be passaged in culture like a standard embryonic stem cell line. Their gene expression patterns and ability to form teratomas indicated that these reprogrammed siPS were substantially identical to induced pluripotent stem cells, and that their generation did not require expression of any stem cell genes or transfer of any mRNA or protein derived from stem cell genes.

Additionally, the developmental potential (e.g., the pluripotency) of siPS was investigated by chimera formation. siPS were injected into mouse blastocysts, where they took part in the development of, and contributed to, cell and tissue types derived from all three primary embryonic germ layers.

### REFERENCES

All references listed in the instant disclosure, including but not limited to all patents, patent applications and publications thereof, scientific journal articles, and database entries (including but not limited to GENBANK® database entries including all annotations available therein) are incorporated herein by reference in their entireties to the extent that they supplement, explain, provide a background for, or teach methodology, techniques, and/or compositions employed herein.
Ajioka et al. (2007) Cell 131, 378-390.
Ansieau et al. (2008) Cancer Cell 14, 79-89.
Assady et al. (2001) Diabetes 50, 1691-1697.
Bader et al. (2000) Circ Res 86, 787-794.
Bagutti et al. (1996) Dev Biol 179, 184-196.
Brustle et al. (1999) Science 285, 754-756.
Burkert et al. (1991) New Biol 3, 698-708.
Buttery et al. (2001) Tissue Eng 7, 89-99.
Cai et al. (2007) Hepatology 45, 1229-1239.
Candi et al. (2007) Cell Cycle 6, 274-285.
Chadwick et al. (2003) Blood 102, 906-915.
D'Amour et al. (2005) Nat Biotechnol 23, 1534-1541.
D'Amour et al. (2006) Nat Biotechnol 24, 1392-1401.
Dalerba et al. (2007) Annu Rev Med 58, 267-84.
Dannenberg & te Riele (2006) Results Probl Cell Differ 42, 183-225.
Dannenberg et al. (2000) Genes Dev 14, 3051-64.
Desbaillets et al. (2000) Exp Physiol 85, 645-651.
Dick (2005) Ann NY Acad Sci 1044, 1-5.
Du & Pogoriler (2006) Oncogene 25, 5190-5200.
Frolov et al. (2004) J Cell Sci 117, 2173-81.
Godar et al. (2008) Cell 134, 62-73.
Harlow & Lane (1988) Antibodies: A Laboratory Manual, Cold Spring Harbor Laboratory, Cold Spring Harbor, N.Y.
Harlow & Lane (1999) Using Antibodies: A Laboratory Manual, Cold Spring Harbor Laboratory, Cold Spring Harbor, N.Y.
Hermann et al. (2004) J Cell Sci 117, 4411-4422.
Hirschmann-Jax et al. (2005) Cell Cycle 4, 203-205.
Itsykson et al. (2005) Mol Cell Neurosci 30, 24-36.
Jaenisch & Young (2008) Cell 132, 567-582.
Jiang et al. (2007) Cell Res 4, 333-444.
Kang et al. (2007) Stem Cells 25, 419-424.
Kansara & Thomas (2007) DNA Cell Biol 26, 1-18.
Kawasaki et al. (2000) Neuron 28, 3140.
Kaye (2002) Oncogene 21, 6908-6914.
Kehat et al. (2001) J Clin Invest 108, 407-414.
Knoblich (2008) Cell 132, 583-597.
Kramer et al. (2000) Mech Dev 92, 193-205.
Lee et al. (2000) Nat Biotechnol 18, 675-679.
Lengner et al. (2007) Cell Stem Cell 1, 403-415.
Lieschke & Dunn (1995) Exp Hemat 23, 328-334.
Liu et al. (2007a) Biochem J408, 79-85.
Liu et al. (2007b) N Engl J Med 356, 217-226.
Liu et al. (2008) Development 235, 579-588.
Liu et al. (2009) Cell Stem Cell 4, 336-347.
Lobo et al. (2007) Annu Rev Cell Dev Biol 23, 675-699.
Lowe & Sherr (2003) Curr Opin Genet Dev 13, 77-83.
Lumelsky et al. (2001) Science 292, 1389-1394.
MacPherson & Dyer (2007) Cancer Res 67, 7547-7550.
Mani et al. (2008) Cell 133, 704-715.
Mummery et al. (2003) Circulation 107, 2733-2740.
Nagy et al. (2003) Manipulating the Mouse Embryo: A Laboratory Manual (Third Edition), Cold Spring Harbor Laboratory Press, Cold Spring Harbor, N.Y., United States of America.
Neth et al. (2007) Stem Cell Rev 3, 18-29.
Nishimura et al. (2007) Dev Cell 11, 93-104.
O'Brien et al. (2007) Nature 225, 106-110.
Okita et al. (2007) Nature 448, 260-262.
Papaioannou & Johnson (2000) in Gene Targeting, A Practical Approach (2^{nd} Edition), Joyner (ed.), Oxford University Press, Oxford, England, United Kingdom, pages 133-176.
PCT International Patent Application Publication Nos. WO 2007/067280; WO 2009/059032.
Peeper et al. (2001) Nat Cell Biol 3, 198-203.
Peinado et al. (2007) Nat Rev Cancer 7, 415-428.
Pomp et al. (2005) Stem Cells 23, 923-930.
Prelle et al. (2000) Biochem Biophys Res Commun 277, 631-638.
Rambhatla et al. (2003) Cell Transplant 12, 1-11.
Reubinoff et al. (2001) Nat Biotechnol 19, 1134-1140.
Robey et al. (2007) Cancer Metastasis Rev 26, 39-57.
Sabado Alvarez (2008) Clin Transl Oncol 10, 389-384.
Saburi et al. (1997) Differentiation 62:1-11.
Sage et al. (2000) Genes Dev 14, 3037-3050.
Sambrook & Russell (2001) Molecular Cloning: A Laboratory Manual, 3rd edition, Cold Spring Harbor Laboratory Press, Cold Spring Harbor, N.Y., United States of America.
Schantz & Ng (2004) A Manual for Primary Human Cell Culture, World Scientific Publishing Co., Inc., Hackensack, N.J., United States of America.
Seigel et al. (2005) Mol Vis 11, 729-37.
Seigel et al. (2007) Mol Vis 13, 823-832.
Sottile et al. (2003) Cloning Stem Cells 5, 149-155.
Stemmer et al. (2008) Oncogene 27, 5075-5080.
Stewart (1993) Methods Enzymol 225:823-855.
Takahashi & Yamanaka (2006) Cell 126, 663-76.
Takahashi et al. (2007) Cell 131, 861-872.
Telang et al. (2006) Oncogene 25, 7225-7234.
Tiscornia et al. (2006) Nat Protoc 1, 234-240
te Riele (2007) Cell 131, 227-229.
Trimbloi et al. (2008) Cancer Res 68, 937-945.
Trosko (2006) Ann NY Acad Sci 1089, 36-58.
Tsai et al. (2000) Proc Natl Acad Sci USA 97, 9186-9190.
U.S. Patent Application Publication Nos. 20090191159; 20090191171.
U.S. Pat. Nos. 5,914,268; 6,395,546; 6,602,711; 7,153,684; 7,220,584.
Wernig et al. (2007) Nature 448, 318-324.
Wong et al. (2008) Cell Stem Cell 2, 333-344.
Yamane et al. (1999) Dev Dyn 216, 450-458.
Yamashita et al. (2000) Nature 408, 92-96.
Yuasa et al. (2005) Nat Biotechnol 23, 607-611.
Zhang & Rosen (2006) Curr Opin Genet Dev 16, 60-64.
Zhang et al. (1999) Cell 97, 53-61.
Zhang et al. (2001) Nat Biotechnol 19, 1129-1133.

It will be understood that various details of the presently disclosed subject matter may be changed without departing from the scope of the presently disclosed subject matter. Furthermore, the foregoing description is for the purpose of illustration only, and not for the purpose of limitation.

What is claimed is:

1. A method for isolating sphere-induced pluripotent cells (siPS), comprising:
(a) growing a plurality of fibroblasts and/or epithelial cells in monolayer culture on a tissue culture plate to confluency; and
(b) disrupting the monolayer culture to place at least a fraction of the plurality of fibroblasts and/or epithelial cells into suspension culture under conditions sufficient to form one or more embryoid body-like spheres;
(c) replating the spheres formed on a fibroblast and/or epithelial cell feeder layer in an embryonic stem cell medium;
(d) culturing the replated spheres on the fibroblast and/or epithelial cell feeder layer in an embryonic stem cell medium for a time sufficient for colonies of undifferentiated siPS derived from the replated spheres to develop; and
(e) isolating the siPS from one or more of the colonies.

2. The method of claim 1, wherein:
(i) the siPS are mouse siPS and the embryonic stem cell medium is a mouse embryonic stem cell medium comprising leukemia inhibitory factor (LIF); or
(ii) the siPS are human siPS and the embryonic stem cell medium is a human embryonic stem cell medium comprising basic epithelial cell growth factor (bFGF).

3. A method for inducing expression of one or more stem cell markers in a reprogrammed fibroblast and/or epithelial cell, the method comprising:
(a) growing a plurality of fibroblasts and/or epithelial cells in monolayer culture to confluency; and
(b) disrupting the monolayer culture to place at least a fraction of the plurality of fibroblasts and/or epithelial cells into suspension culture under conditions sufficient to form one or more spheres,
wherein the one or more spheres comprise a reprogrammed fibroblast and/or epithelial cell expressing one or more stem cell markers.

4. The method of claim 3, further comprising replating the spheres formed under conditions sufficient for one or more reprogrammed fibroblasts and/or epithelial cells present therein to form one or more colonies.

5. The method of claim 4, wherein the conditions sufficient for one or more reprogrammed fibroblasts and/or epithelial cells present therein to form colonies comprise culturing the replated spheres in the presence of an embryonic stem cell medium at least until one or more cells derived from the replated spheres form one or more colonies.

6. A method for producing a chimeric non-human mammal, the method comprising transferring one or more sphere-induced Pluripotent Cells (siPS) produced from cells isolated from a non-human mammal into a host embryo and implanting the host embryo into a recipient female, wherein a chimeric non-human mammal comprising one or more somatic and/or germ cells is a progeny cell of one or more of the siPS transferred into the host embryo is produced.

7. The method of claim 6, wherein the non-human mammal is a mouse.

8. The method of claim 6, wherein the one or more siPS is produced by:
(a) growing a plurality of fibroblasts or epithelial cells in monolayer culture to confluency; and
(b) disrupting the monolayer culture to place at least a fraction of the plurality of fibroblasts or epithelial cells into suspension culture under conditions sufficient to form one or more embryoid body-like spheres.

9. The method of claim 8, wherein the fibroblasts or epithelial cells are mouse fibroblasts or epithelial cells.

10. The method of claim 8, wherein the disrupting comprises scraping the confluent monolayer off of a substrate upon which the confluent monolayer is being cultured.

11. The method of claim 8, further comprising maintaining the one or more embryoid body-like spheres in suspension culture for at least one month.

12. The method of claim 11, wherein the one or more embryoid body-like spheres are maintained in a medium comprising DMEM and 10% FBS.

13. The method of claim 8, further comprising replating the embryoid body-like spheres under conditions sufficient for at least a subset of the cells present therein to form colonies.

14. The method of claim 13, wherein the conditions sufficient comprise plating the embryoid body-like spheres on a fibroblast or epithelail feeder layer in an embryonic stem cell medium until colonies of sphere-induced Pluripotent Cells (siPS) are produced.

15. The method of claim 14, further comprising subcloning one or more cells present in a colony of siPS to form one or more siPS cell lines.

16. The method of claim 8, wherein the one or more embryoid body-like spheres comprise a reprogrammed fibroblast or epithelial cell induced to express at least one endogenous gene not expressed by a fibroblast or epithelial cell growing in the monolayer culture prior to the disrupting step.

17. The method of claim 16, wherein the reprogrammed fibroblast or epithelial cell expresses at least one endogenous gene selected from the group consisting of Oct4, Nanog, FGF4, Sox2, Klf4, Ssea1, and Stat3.

18. The method of claim 8, wherein the fibroblast or epithelial cell comprises at least one transgene.

19. The method of claim 18, wherein the transgene is operably linked to a promoter that is active in at least one cell type and/or developmental stage of the chimeric non-human mammal to an extent sufficient to modify a phenotype of the chimeric non-human mammal as compared to a non-chimeric non-human mammal of the same genetic background and/or species as that of the host embryo.

20. The method of claim 6, wherein the transferring comprises transferring at least s siPS into the host embryo and/or the implanting comprises implanting the host embryo into a pseudopregnant female.

21. The method of claim 6, wherein the host embryo is a morula stage embryo or a blastocyst stage embryo.

22. A chimeric non-human mammal produced by the method of claim 6.

23. The chimeric non-human mammal of claim 22, wherein the chimeric non-human mammal is a mouse.

24. The chimeric non-human mammal of claim 22, wherein the chimeric non-human mammal is a pre-term embryo.

25. The chimeric non-human mammal of claim 22, wherein one or more sphere-induced Pluripotent Cells (siPS)-derived cells are present within the germline of the chimeric non-human mammal, thereby producing a germline chimeric non-human mammal.

26. A method for producing a reprogrammed epithelial cell, the method comprising:
(a) growing a plurality of epithelial cells in monolayer culture to confluency; and
(b) disrupting the monolayer culture to place at least a fraction of the plurality of epithelial cells into suspension culture under conditions sufficient to form one or more embryoid body-like spheres,
wherein the one or more embryoid body-like spheres comprise a reprogrammed epithelial cell induced to express at least one endogenous gene not expressed by a epithelial cell growing in the monolayer culture prior to the disrupting step.

27. The method of claim 26, wherein the epithelial cell is a mouse epithelial cell or a human epithelial cell.

28. The method of claim 26, wherein the epithelial cell is a non-recombinant epithelial cell.

29. The method of claim 26, wherein the disrupting comprises scraping the confluent monolayer off of a substrate upon which the confluent monolayer is being cultured.

30. The method of claim 26, further comprising maintaining the one or more embryoid body-like spheres in suspension culture for at least one month.

31. The method of claim 30, wherein the one or more embryoid body-like spheres are maintained in a medium comprising DMEM and 10% FBS.

32. The method of claim 26, wherein the reprogrammed epithelial cell expresses at least one endogenous gene is selected from the group consisting of Oct4, Nanog, FGF4, Sox2, Klf4, Ssea1 , and Stat3.

33. The method of claim 26, further comprising replating the embryoid body-like spheres under conditions sufficient for the reprogrammed epithelial cells present therein to form colonies.

34. The method of claim 33, wherein the conditions sufficient comprise plating the embryoid body-like spheres on a epithelial cell feeder layer in an embryonic stem cell medium until colonies of Sphere-induced Pluripotent Cells (siPS) are produced.

35. The method of claim 33, further comprising subcloning one or more cells present in a colony of reprogrammed epithelial cells to form one or more Sphere-induced Pluripotent Cell (siPS) lines.

36. A reprogrammed epithelial cell produced by the method of claim 26.

37. A formulation comprising the reprogrammed epithelial cell cell of claim 36 in a pharmaceutically acceptable carrier or excipient.

38. The formulation of claim 37, wherein the pharmaceutically acceptable carrier or excipient is acceptable for use in humans.

39. A cell culture comprising an embryoid body-like sphere produced by the method of claim 26 in a medium sufficient to maintain the embryoid body-like sphere in suspension culture for at least one month.

40. A cell culture comprising the reprogrammed epithelial cell of claim 36 in a medium sufficient to maintain the reprogrammed epithelial cell in an undifferentiated state for at least one month.


SEQUENCE LISTING (74 sequences)

SEQ ID NO: 1 - DNA, 19 bp, Artificial sequence
  Artificially synthesized oligonucleotide
agtggcgtgctgtgttgag

SEQ ID NO: 2 - DNA, 21 bp, Artificial sequence
  Artificially synthesized oligonucleotide
aacaatagggaccagcccatt

SEQ ID NO: 3 - DNA, 21 bp, Artificial sequence
  Artificially synthesized oligonucleotide
gtcccagacatcagggagtaa

SEQ ID NO: 4 - DNA, 21 bp, Artificial sequence
  Artificially synthesized oligonucleotide
tcggatacttcagcgtcagga

SEQ ID NO: 5 - DNA, 19 bp, Artificial sequence
  Artificially synthesized oligonucleotide
gtggatgcagccactctag

SEQ ID NO: 6 - DNA, 20 bp, Artificial sequence
  Artificially synthesized oligonucleotide
ttagccgcgatggtctcata

SEQ ID NO: 7 - DNA, 21 bp, Artificial sequence
  Artificially synthesized oligonucleotide
aaggctgggtgaagaccctta

SEQ ID NO: 8 - DNA, 21 bp, Artificial sequence
  Artificially synthesized oligonucleotide
tgaatggccgtttctggaagt

SEQ ID NO: 9 - DNA, 20 bp, Artificial sequence
  Artificially synthesized oligonucleotide
caagcatagtggtccgagtc

SEQ ID NO: 10 - DNA, 20 bp, Artificial sequence
  Artificially synthesized oligonucleotide
aggcaggtcaagttctagcg

SEQ ID NO: 11 - DNA, 21 bp, Artificial sequence
  Artificially synthesized oligonucleotide
caccccaatctcgatatgttt

SEQ ID NO: 12 - DNA, 21 bp, Artificial sequence
  Artificially synthesized oligonucleotide
ggttgatgccgttcatcttgt

SEQ ID NO: 13 - DNA, 20 bp, Artificial sequence
  Artificially synthesized oligonucleotide
aagtgactgtgaaaacagaa

SEQ ID NO: 14 - DNA, 21 bp, Artificial sequence
  Artificially synthesized oligonucleotide
gcagccatttgtaagggttga

SEQ ID NO: 15 - DNA, 20 bp, Artificial sequence
  Artificially synthesized oligonucleotide
gaaaggaagacccgaagaaa

SEQ ID NO: 16 - DNA, 21 bp, Artificial sequence
  Artificially synthesized oligonucleotide
ccatagggctaggacaccaaa

SEQ ID NO: 17 - DNA, 21 bp, Artificial sequence
  Artificially synthesized oligonucleotide
aactggcacacctcaagatgt

SEQ ID NO: 18 - DNA, 21 bp, Artificial sequence
  Artificially synthesized oligonucleotide
tcaagggtattaggcaagggg

SEQ ID NO: 19 - DNA, 20 bp, Artificial sequence
  Artificially synthesized oligonucleotide
tcctccactcaaccattctt

SEQ ID NO: 20 - DNA, 20 bp, Artificial sequence
  Artificially synthesized oligonucleotide
tcaaggcagagcagttcata

SEQ ID NO: 21 - DNA, 21 bp, Artificial sequence
  Artificially synthesized oligonucleotide
ggatcttcatggtgaatgtca

SEQ ID NO: 22 - DNA, 21 bp, Artificial sequence
  Artificially synthesized oligonucleotide
ctctggttgctcctgttctca

SEQ ID NO: 23 - DNA, 21 bp, Artificial sequence
  Artificially synthesized oligonucleotide
gacttcgaggcgacacttcta

SEQ ID NO: 24 - DNA, 22 bp, Artificial sequence
  Artificially synthesized oligonucleotide
gttgaagaggaaacgaaaagca

SEQ ID NO: 25 - DNA, 20 bp, Artificial sequence
  Artificially synthesized oligonucleotide
tcttccccaacggtactatc

SEQ ID NO: 26 - DNA, 19 bp, Artificial sequence
  Artificially synthesized oligonucleotide
ccgaggtagagtccactgt

SEQ ID NO: 27 - DNA, 20 bp, Artificial sequence
  Artificially synthesized oligonucleotide
agttggcgtggagactttgc

SEQ ID NO: 28 - DNA, 19 bp, Artificial sequence
  Artificially synthesized oligonucleotide
cagggctttcatgtcctgg

SEQ ID NO: 29 - DNA, 21 bp, Artificial sequence
  Artificially synthesized oligonucleotide
gttgagactgtgcccatgaaa

SEQ ID NO: 30 - DNA, 21 bp, Artificial sequence
  Artificially synthesized oligonucleotide
gacgggcttgtcataacagga

SEQ ID NO: 31 - DNA, 19 bp, Artificial sequence
  Artificially synthesized oligonucleotide
cctctcacggcttatgggc

SEQ ID NO: 32 - DNA, 19 bp, Artificial sequence
  Artificially synthesized oligonucleotide
ctgtggcaatcaagggacc

SEQ ID NO: 33 - DNA, 22 bp, Artificial sequence
  Artificially synthesized oligonucleotide
tctgccatctagcactaagagc

SEQ ID NO: 34 - DNA, 23 bp, Artificial sequence
  Artificially synthesized oligonucleotide
gtctgggtattgaaaggtgtagc

SEQ ID NO: 35 - DNA, 21 bp, Artificial sequence
  Artificially synthesized oligonucleotide
acccacgcagatttactgcaa

SEQ ID NO: 36 - DNA, 20 bp, Artificial sequence
  Artificially synthesized oligonucleotide
cccctctggtggtagcgtta

SEQ ID NO: 37 - DNA, 19 bp, Artificial sequence
  Artificially synthesized oligonucleotide
tgtgaggacgtagagacgg

SEQ ID NO: 38 - DNA, 22 bp, Artificial sequence
  Artificially synthesized oligonucleotide
gcagcacgacgttcttaatgtc

SEQ ID NO: 39 - DNA, 22 bp, Artificial sequence
  Artificially synthesized oligonucleotide
ttgcttacaagggtctgctact

SEQ ID NO: 40 - DNA, 21 bp, Artificial sequence
  Artificially synthesized oligonucleotide
actggtagaagaatcagggct

SEQ ID NO: 41 - DNA, 21 bp, Artificial sequence
  Artificially synthesized oligonucleotide
gcggagtggaaacttttgtcc

SEQ ID NO: 42 - DNA, 22 bp, Artificial sequence
  Artificially synthesized oligonucleotide
cgggaagcgtgtacttatcctt

SEQ ID NO: 43 - DNA, 19 bp, Artificial sequence
  Artificially synthesized oligonucleotide
agctggacacacgctacct

SEQ ID NO: 44 - DNA, 22 bp, Artificial sequence
  Artificially synthesized oligonucleotide
aggaatcggctatattgctggt

SEQ ID NO: 45 - DNA, 21 bp, Artificial sequence
  Artificially synthesized oligonucleotide
aggaggcagcagttattgtgg

SEQ ID NO: 46 - DNA, 21 bp, Artificial sequence
  Artificially synthesized oligonucleotide
cgttgaccttagtacccagga

SEQ ID NO: 47 - DNA, 20 bp, Artificial sequence
  Artificially synthesized oligonucleotide
ggctgtattcccctccatcg

SEQ ID NO: 48 - DNA, 22 bp, Artificial sequence
  Artificially synthesized oligonucleotide
ccagttggtaacaatgccatgt

SEQ ID NO: 49 - DNA, 21 bp, Artificial sequence
  Artificially synthesized oligonucleotide
aggtcggtgtgaacggatttg

SEQ ID NO: 50 - DNA, 23 bp, Artificial sequence
  Artificially synthesized oligonucleotide
tgtagaccatgtagttgaggtca

SEQ ID NO: 51 - DNA, 19 bp, Artificial sequence
  Artificially synthesized oligonucleotide
gggcagaattacccacgca

SEQ ID NO: 52 - DNA, 19 bp, Artificial sequence
  Artificially synthesized oligonucleotide
ctggcgagaaatgacgcaa

SEQ ID NO: 53 - DNA, 21 bp, Artificial sequence
  Artificially synthesized oligonucleotide
acaccttgggacacggttttc

SEQ ID NO: 54 - DNA, 21 bp, Artificial sequence
  Artificially synthesized oligonucleotide
taggtcttgttcctcggccat

SEQ ID NO: 55 - DNA, 24 bp, Artificial sequence
  Artificially synthesized oligonucleotide
agtcgtatctggccatggcttctt

SEQ ID NO: 56 - DNA, 24 bp, Artificial sequence
  Artificially synthesized oligonucleotide
acagcaagctgtggtagtcgtctt

SEQ ID NO: 57 - DNA, 24 bp, Artificial sequence
  Artificially synthesized oligonucleotide
atactgggaccagatggcaacaca

SEQ ID NO: 58 - DNA, 24 bp, Artificial sequence
  Artificially synthesized oligonucleotide
aagcgggtccttcgtgagagaaat

SEQ ID NO: 59 - DNA, 24 bp, Artificial sequence
  Artificially synthesized oligonucleotide
tggatctctgttgctggaaagggt

SEQ ID NO: 60 - DNA, 24 bp, Artificial sequence
  Artificially synthesized oligonucleotide
aggctgaggagccttcatagcatt

SEQ ID NO: 61 - DNA, 24 bp, Artificial sequence
  Artificially synthesized oligonucleotide
ttgatggatccggccttgcaaatg

SEQ ID NO: 62 - DNA, 24 bp, Artificial sequence
  Artificially synthesized oligonucleotide
tatgttgggaaggttggctggaca

SEQ ID NO: 63 - DNA, 20 bp, Artificial sequence
  Artificially synthesized oligonucleotide
ttcacgaagaacccaaaacc

SEQ ID NO: 64 - DNA, 20 bp, Artificial sequence
  Artificially synthesized oligonucleotide
agttgctggcgtagcaagat

SEQ ID NO: 65 - DNA, 21 bp, Artificial sequence
  Artificially synthesized oligonucleotide
gatggaggcgcttagatttga

SEQ ID NO: 66 - DNA, 20 bp, Artificial sequence
  Artificially synthesized oligonucleotide
catgagttgctggcgtagca

SEQ ID NO: 67 - DNA, 19 bp, Artificial sequence
  Artificially synthesized oligonucleotide
gctggaaatgctagaatac

SEQ ID NO: 68 - DNA, 20 bp, Artificial sequence
  Artificially synthesized oligonucleotide
ggctggcatgtttatttgct

SEQ ID NO: 69 - DNA, 20 bp, Artificial sequence
  Artificially synthesized oligonucleotide
ggctgtattcccctccatcg

SEQ ID NO: 70 - DNA, 22 bp, Artificial sequence
  Artificially synthesized oligonucleotide
ccagttggtaacaatgccatgt

SEQ ID NO: 71 - DNA, 21 bp, Artificial sequence
  Artificially synthesized oligonucleotide
atttaggtgacactatagaat

SEQ ID NO: 72 - DNA, 19 bp, Artificial sequence
  Artificially synthesized oligonucleotide
aagacaacgtgaaagacaa

SEQ ID NO: 73 - DNA, 19 bp, Artificial sequence
  Artificially synthesized oligonucleotide
ggaaaaacgtggtgaacta

SEQ ID NO: 74 - DNA, 19 bp, Artificial sequence
  Artificially synthesized oligonucleotide
aacaagatgaagagcacca